(19) United States
(12) Patent Application Publication
(10) Pub. No.: US 2025/0127939 A1
(43) Pub. Date: Apr. 24, 2025

(54) COMPOUNDS FOR POSITRON EMISSION TOMOGRAPHY

(71) Applicants: Endocyte, Inc., East Hanover, NJ (US); Purdue Research Foundation, West Lafayette, IN (US)

(72) Inventors: Iontcho R. VLAHOV, West Lafayette, IN (US); Christopher P. LEAMON, West Lafayette, IN (US); Philip S. LOW, West Lafayette, IN (US); Garth L. PARHAM, Largo, FL (US); Qingshou CHEN, West Lafayette, IN (US)

(21) Appl. No.: 18/913,734

(22) Filed: Oct. 11, 2024

Related U.S. Application Data
(63) Continuation of application No. 17/488,810, filed on Sep. 29, 2021, now Pat. No. 12,178,892.
Application No. 17/488,810 is a continuation of application No. 16/157,024, filed on Oct. 10, 2018, now abandoned.
Application No. 16/157,024 is a continuation of application No. 15/035,936, filed on May 11, 2016, now abandoned.
(60) Provisional application No. 61/904,400, filed on Nov. 14, 2013.
(60) Provisional application No. 61/904,387, filed on Nov. 14, 2013.
(60) Provisional application No. 61/909,822, filed on Nov. 27, 2013.

(51) Int. Cl. A61K 51/04

(52) U.S. Cl. CPC A61K 51/0497; A61K 51/04; A61K 51/0459; A61K 51/0482

(57) ABSTRACT

Described herein are compounds, compositions, and methods for diagnosing and/or monitoring pathogenic disease using positron emission tomography.

### CROSS-REFERENCE TO RELATED APPLICATIONS

This application is a continuation of U.S. application Ser. No. 17/488,810, filed Sep. 29, 2021, which is a continuation of U.S. application Ser. No. 16/157,024, filed Oct. 10, 2018, which is a continuation of U.S. application Ser. No. 15/035,936, filed May 11, 2016, which is the national stage filing under 35 U.S.C. § 371 of International Application No. PCT/US2014/0650467, filed Nov. 13, 2014, which claims the benefit under 35 U.S.C. § 119 (e) to U.S. Provisional Application Ser. No. 61/904,387, filed Nov. 14, 2013, 61/904,400, filed Nov. 14, 2013, and 61/909,822, filed Nov. 27, 2013, the disclosure of each of which is incorporated herein by reference in its entirety.

### TECHNICAL FIELD

The invention described herein pertains to compounds, compositions, and methods for diagnosing and/or monitoring diseases and disease states using radionuclides. In particular, the invention described herein pertains to compounds, compositions, and methods for diagnosing and/or monitoring pathogenic disease states using radionuclides for positron emission tomography (PET).

### BACKGROUND AND SUMMARY OF THE INVENTION

PET is a nuclear imaging methodology that detects pairs of gamma rays emitted indirectly by a positron-producing radionuclide. Because the two emitted gamma rays travel in exactly opposite directions, it is possible to locate their site of origin and thereby reconstruct a three-dimensional image of all positron emitters from a computer analysis of the origins of emitted gamma rays. Compared to other radioimaging modalities, such as SPECT, PET reportedly shows higher sensitivity (about 2 orders of magnitude), better spatial resolution (about 5 mm), greater signal to noise, and superior tracer quantification in both preclinical and clinical applications. In addition, in contrast to the about 90 minutes required for body scans for a standard SPECT imaging, PET image acquisition may be routinely performed in about 20 minutes. Moreover, in vivo PET imaging generally requires only subnanomolar (10⁻¹⁰ to 10⁻¹²) concentrations of radiotracer, which reportedly minimizes potential damage to other biological systems. Finally, PET allows for quantitative dynamic imaging, which may facilitate kinetic studies of target engagement through receptor occupancy. It has been discovered herein that PET agents may be targeted to predetermined tissues using vitamin receptors and/or prostate-specific membrane antigen (PSMA).

For example, vitamin receptors are overexpressed on certain pathogenic cells, including many cancer cell types, activated macrophages, and activated monocytes. In particular, folate receptors are overexpressed in many cancers. The folate receptor, a 38 KD GPI-anchored protein that binds the vitamin folic acid with high affinity (<1 nM), is overexpressed on many malignant tissues, including ovarian, breast, bronchial, and brain cancers. It is estimated that 95% of all ovarian carcinomas overexpress the folate receptor. In contrast, with the exception of kidney, choroid plexus, and placenta, normal tissues express low or nondetectable levels of the folate receptor. Most cells also use an unrelated reduced folate carrier to acquire the necessary folic acid.

Folate receptors are also overexpressed on activated macrophages, and activated monocytes. Further, it has also been reported that the folate receptor B, the nonepithelial isoform of the folate receptor, is expressed on activated, but not resting, synovial macrophages. Activated macrophages can participate in the immune response by nonspecifically engulfing and killing foreign pathogens within the macrophage, by displaying degraded peptides from foreign proteins on the macrophage cell surface where they can be recognized by other immune cells, and by secreting cytokines and other factors that modulate the function of T and B lymphocytes, resulting in further stimulation of immune responses. However, activated macrophages can also contribute to the pathophysiology of disease in some instances. For example, activated macrophages can contribute to atherosclerosis, rheumatoid arthritis, autoimmune disease states, and graft versus host disease, among other disease states.

Following receptor binding of vitamins to vitamin receptors, such as folic acid and analogs and derivatives of folic acid to folate receptors, rapid endocytosis delivers the vitamin into the cell, where it is unloaded in an endosomal compartment at lower pH. Importantly, covalent conjugation of small molecules, proteins, and even liposomes to vitamins and other vitamin receptor binding ligands does not block the ability of the ligand to bind to its receptor, and therefore, such ligand conjugates can readily be delivered to and can enter cells by receptor-mediated endocytosis. Accordingly, diagnostic, imaging, and therapeutic agents can be targeted to vitamin receptors, including the folate receptor, for delivery into vitamin receptor expressing cells.

The prostate is a male reproductive organ that functions to produce and store seminal fluid, which provides nutrients and fluids for the survival of sperm introduced into the vagina during reproduction. Like other tissues, the prostate gland may develop either malignant (cancerous) or benign (non-cancerous) tumors. Prostate cancer is reportedly one of the most common male cancers in western societies, and is the second leading form of malignancy among American men.

Prostate-specific membrane antigen (PSMA) is a biomarker that is overexpressed on prostate cancer. PSMA is over-expressed in the malignant prostate tissues when compared to other organs in the human body such as kidney, proximal small intestine, and salivary glands. PSMA is also expressed on the neovasculature within many non-prostate solid tumors, including lung, colon, breast, renal, liver and pancreatic carcinomas, but not on normal vasculature. However, PSMA is expressed minimally in brain. PSMA is a type II cell surface membrane-bound glycoprotein with ˜110 kD molecular weight, including an intracellular segment (amino acids 1-18), a transmembrane domain (amino acids 19-43), and an extensive extracellular domain (amino acids 44-750). Though the functions of the intracellular segment and the transmembrane domains are currently reported to be insignificant, the extracellular domain is involved in several distinct activities. For example, PSMA plays a role in the central nervous system, where it metabolizes N-acetyl-aspartyl glutamate (NAAG) into glutamic and N-acetyl aspartic acid. PSMA also plays a role in the proximal small intestine where it removes γ-linked glutamate from poly-γ-glutamated folate and α-linked glutamate from peptides and small molecules.

Though the particular function of PSMA on prostate cancer cells remains unresolved, PSMA is known to undergo rapid internalization into the cell, similar to cell surface bound receptors like vitamin receptors. PSMA is internalized through clathrin-coated pits and subsequently can either recycle to the cell surface or go to lysosomes. Accordingly, diagnostic, imaging, and therapeutic agents can be targeted to PSMA for delivery into PSMA expressing cells, such as prostate cancer cells.

It has been discovered herein that the compounds and compositions described herein are useful for targeting and delivering radionuclides for diagnosing and/or monitoring various diseases and disease states caused by pathogenic cell populations. In addition, it has been discovered that the compounds and compositions described herein are also useful for targeting and delivering radionuclides for treating various diseases and disease states caused by pathogenic cell populations in radiotherapy.

In one illustrative and non-limiting embodiment of the invention described herein, compounds and compositions described herein are used for diagnosing and/or monitoring, or treating various diseases and disease states caused by pathogenic cell populations. In another illustrative embodiment, methods are described herein for administering compounds and compositions described herein for diagnosing and/or monitoring, or treating various diseases and disease states caused by pathogenic cell populations. In another embodiment, uses of compounds and compositions are described herein for manufacturing medicaments for diagnosing and/or monitoring, or treating various diseases and disease states caused by pathogenic cell populations. In another embodiment, kits are described herein for preparing and/or using compounds and compositions described herein for diagnosing and/or monitoring, or treating various diseases and disease states caused by pathogenic cell populations.

### BRIEF DESCRIPTION OF THE DRAWINGS

FIG. 1A shows a postmortem biodistribution study of ¹⁸F-AIF-QC07017 and ¹⁸F-AIF-QC07043 folate-NOTA-A1-¹⁸F conjugates in various tissues at 90 minutes post injection in nude mice bearing KB tumor xenografts. For each tissue, the histogram is in groups of 4 from left to right: ¹⁸F-AIF-QC07017, 18F-AIF-QC07017+excess folic acid, ¹⁸F-AIF-QC07043, 18F-AIF-QC07043+excess folic acid.

FIG. 1B shows a postmortem biodistribution study of ¹⁸F-AIF-QC07017 folate-NOTA-A1-¹⁸F conjugate in various tissues at 90 minutes post injection in nude mice bearing KB tumor xenografts or A549 tumor xenografts. It is to be understood that the vertical axis has been expanded and that the kidney data is truncated. For each tissue, the histogram is in groups of 4 from left to right: ¹⁸F-AIF-QC07017 against A549 tumor xenografts, ¹⁸F-AIF-QC07017+excess folic acid against A549 tumor xenografts, ¹⁸F-AIF-QC07017 against KB tumor xenografts, ¹⁸F-AIF-QC07017+excess folic acid against KB tumor xenografts.

FIG. 1C shows a postmortem biodistribution study of ¹⁸F-AIF-QC07043 folate-NOTA-A1-¹⁸F conjugate in various tissues at 90 minutes post injection in nude mice bearing KB tumor xenografts or A549 tumor xenografts. It is to be understood that the vertical axis has been expanded and that the kidney data is truncated. For each tissue, the histogram is in groups of 4 from left to right: ¹⁸F-AIF-QC07043 against A549 tumor xenografts, ¹⁸F-AIF-QC07043+excess folic acid against A549 tumor xenografts, ¹⁸F-AIF-QC07043 against KB tumor xenografts, ¹⁸F-AIF-QC07043+excess folic acid against KB tumor xenografts.

FIG. 2A shows a postmortem biodistribution study of ¹⁸F-AIF-QC07017 and ¹⁸F-AIF-QC07043 folate-NOTA-A1-¹⁸F conjugates, compared to ^{99m}Tc-EC20 in KB tumor xenograft tissues at 90 minutes post injection in nude mice. The histogram from left to right: ^{99m}Tc-EC20 against KB tumor xenografts, ^{99m}Tc-EC20+excess folic acid against KB tumor xenografts, ¹⁸F-AIF-QC07017 against KB tumor xenografts, ¹⁸F-AIF-QC07017+excess folic acid against KB tumor xenografts, ¹⁸F-AIF-QC07043 against KB tumor xenografts, ¹⁸F-AIF-QC07043+excess folic acid against KB tumor xenografts.

FIG. 2B shows a postmortem biodistribution study of ¹⁸F-AIF-QC07017 and ¹⁸F-AIF-QC07043 folate-NOTA-A1-¹⁸F conjugates, compared to ^{99m}Tc-EC20 in A549 tumor xenograft tissues at 90 minutes post injection in nude mice. The histogram from left to right: ^{99m}Tc-EC20 against A549 tumor xenografts, ^{99m}Tc-EC20+excess folic acid against A549 tumor xenografts, ¹⁸F-AIF-QC07017 against A549 tumor xenografts, ¹⁸F-AIF-QC07017+excess folic acid against A549 tumor xenografts, ¹⁸F-AIF-QC07043 against A549 tumor xenografts, ¹⁸F-AIF-QC07043+excess folic acid against A549 tumor xenografts.

### DETAILED DESCRIPTION

In each of the foregoing and each of the following embodiments, it is to be understood that the formulae include and represent not only all pharmaceutically acceptable salts of the compounds, but also include any and all hydrates and/or solvates of the compound formulae. It is appreciated that certain functional groups, such as the hydroxy, amino, and like groups form complexes and/or coordination compounds with water and/or various solvents, in the various physical forms of the compounds. Accordingly, the formulae described herein are to be understood to include and represent those various hydrates and/or solvates. It is also to be understood that the non-hydrates and/or non-solvates of the compound formulae are described by such formula, as well as the hydrates and/or solvates of the compound formulae.

As used herein, the term “composition” generally refers to any product comprising the specified ingredients in the specified amounts, as well as any product which results, directly or indirectly, from combinations of the specified ingredients in the specified amounts. It is to be understood that the compositions described herein may be prepared from isolated compounds described herein or from salts, solutions, hydrates, solvates, and other forms of the compounds described herein. It is appreciated that certain functional groups, such as the hydroxy, amino, and like groups form complexes and/or coordination compounds with water and/or various solvents, in the various physical forms of the compounds. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein. In addition, it is to be understood that the compositions may be prepared from various co-crystals of the compounds described herein.

Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21st ed., 2005)).

In each of the foregoing and each of the following embodiments, it is also to be understood that the formulae include and represent each possible isomer, such as stereoisomers and geometric isomers, both individually and in any and all possible mixtures. In each of the foregoing and each of the following embodiments, it is also to be understood that the formulae include and represent any and all crystalline forms, partially crystalline forms, and non crystalline and/or amorphous forms of the compounds.

Illustrative embodiments of the invention are described by the following clauses:

A conjugate of the formula

B-L-P

or a pharmaceutically acceptable salt thereof, wherein B is a radical of a targeting agent selected from vitamin receptor binding ligands, PSMA binding ligands, and PSMA inhibitors, L is a divalent linker, and P is a radical of an imaging agent or radiotherapy agent, such as a radionuclide or radionuclide containing group, or a precursor thereof, or a radical of a compound capable of binding a radionuclide or radionuclide containing group, such as a metal chelating group.

The conjugate of the preceding clause wherein the targeting agent is a radical of a folate receptor binding ligand.

The conjugate of any one of the preceding clauses wherein the targeting agent is a radical of a folic acid.

The conjugate of any one of the preceding clauses comprising folate-Asp.

The conjugate of any one of the preceding clauses comprising folate-Asp-Arg.

The conjugate of any one of the preceding clauses comprising folate-Arg.

The conjugate of any one of the preceding clauses wherein the linker comprises a polypeptide.

The conjugate of any one of the preceding clauses wherein the linker comprises a polypeptide comprising lysine, arginine, or aspartic acid, or a combination thereof. The conjugate of any one of the preceding clauses wherein the linker comprises a lysine.

The conjugate of any one of the preceding clauses wherein the linker comprises Lys.

The conjugate of any one of the preceding clauses wherein the linker comprises Arg-Lys.

The conjugate of any one of the preceding clauses wherein the linker comprises Arg-Arg-Lys.

The conjugate of any one of the preceding clauses wherein the linker comprises Asp-Arg-Arg-Lys.

The conjugate of any one of the preceding clauses wherein the linker does not include a polyamine radical, such as a polyamine diradical of the formula NH—(CH2)₂—NH.

The conjugate of any one of the preceding clauses wherein P comprises the formula

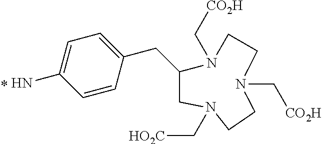 [SMILES: Nc1ccc(CC2CN(CC(=O)O)CCN(CC(=O)O)CCN2CC(=O)O)cc1]

or a derivative thereof comprising a chelated metal.

The conjugate of any one of the preceding clauses comprising the formula

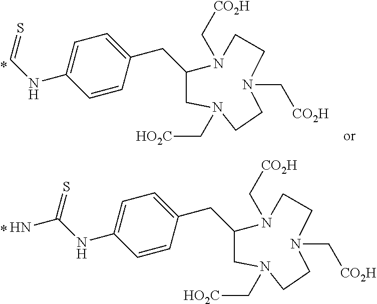

or a derivative thereof comprising a chelated metal.

The conjugate of any one of the preceding clauses comprising folate-PEG.

The conjugate of any one of the preceding clauses comprising folate-PEG₂.

The conjugate of any one of the preceding clauses comprising folate-PEG₆.

The conjugate of any one of the preceding clauses comprising folate-PEG₁₂.

The conjugate of any one of the preceding clauses wherein the linker comprises [(CH₂)₂O]ₙ, [(CH₂)₂O]ₙ—(CH₂)₂—C(O), [(CH₂)₂O]ₙ—(CH₂)₂—C(O)NH, [(CH₂)₂O]ₙ—(CH₂)₂—C(O)NH—(CH₂)₂, [(CH₂)₂]O₂—(CH₂)ₙ-C(O)NH—(CH₂)₂NH, where n is an integer from 1 to about 12.

The conjugate of any one of the preceding clauses wherein the linker comprises [(CH₂)₂O]₂, [(CH₂)₂₀₁₆, or [(CH₂)₂O]₁₂.

The conjugate of any one of the preceding clauses wherein the linker comprises (CH₂)₂O—(CH₂)₂—C(O), [(CH₂)₂O]₂—(CH₂)₂—C(O), [(CH₂)₂₀₁₆—(CH₂)₂—C(O), or [(CH₂)₂O]₁₂—(CH₂)₂—C(O).

The conjugate of any one of the preceding clauses wherein the linker comprises (CH₂)₂O—(CH₂)₂—C(O)NH, [(CH₂)₂O]₂—(CH₂)₂—C(O)NH, [(CH₂)₂₀₁₆—(CH₂)₂—C(O)NH, or [(CH₂)₂O]₁₂—(CH₂)₂—C(O)NH.

The conjugate of any one of the preceding clauses wherein the linker comprises (CH₂)₂O—(CH₂)₂—C(O)NH—(CH₂)₂, [(CH₂)₂O]₂—(CH₂)₂—C(O)NH—(CH₂)₂, [(CH₂)₂O]₆—(CH₂)₂—C(O)NH—(CH₂)₂, or [(CH₂)₂O]₁₂—(CH₂)₂—C(O)NH—(CH₂)₂.

The conjugate of any one of the preceding clauses wherein the linker comprises (CH₂)₂O—(CH₂)₂—C(O)NH—(CH₂)₂NH, [(CH₂)₂O]₂—(CH₂)₂—C(O)NH—(CH₂)₂NH, [(CH₂)₂O]₆—(CH₂)₂—C(O)NH—(CH₂)₂NH, or [(CH₂)₂O]₁₂—(CH₂)₂—C(O)NH—(CH₂)₂NH.

The conjugate of any one of the preceding clauses wherein the linker comprises NH[(CH₂)₂O]ₙ, NH[(CH₂)₂O]ₙ—(CH₂)₂—C(O), NH[(CH₂)₂O]ₙ—(CH₂)₂—C(O)NH, NH[(CH₂)₂O]ₙ—(CH₂)₂—C(O)NH—(CH₂)₂, NH[(CH₂)₂O]ₙ—(CH₂)₂—C(O)NH—(CH₂)₂NH, where n is an integer from 1 to about 12.

The conjugate of any one of the preceding clauses wherein the linker comprises NH(CH₂)₂₀, NH[(CH₂)₂₀₁₂, NH[(CH₂)₂₀₁₆, NH[(CH₂)₂O]₁₂.

The conjugate of any one of the preceding clauses wherein the linker comprises NH(CH₂)₂O—(CH₂)₂—C(O), NH[(CH₂)₂O]₂—(CH₂)₂—C(O), NH[(CH₂)₂O]₆—(CH₂)₂—C(O), or NH[(CH₂)₂O]₁₂—(CH₂)₂—C(O).

The conjugate of any one of the preceding clauses wherein the linker comprises NH(CH₂)₂O—(CH₂)₂—C(O)NH, NH[(CH₂)₂O]₂—(CH₂)₂—C(O)NH, NH[(CH₂)₂O]₆—(CH₂)₂—C(O)NH, or NH[(CH₂)₂O]₁₂—(CH₂)₂—C(O)NH.

The conjugate of any one of the preceding clauses wherein the linker comprises NH(CH₂)₂O—(CH₂)₂—C(O)NH—(CH₂)₂, NH[(CH₂)₂O]₂—(CH₂)₂—C(O)NH—(CH₂)₂, NH[(CH₂)₂O]₆—(CH₂)₂—C(O)NH—(CH₂)₂, or NH[(CH₂)₂O]₁₂—(CH₂)₂—C(O)NH—(CH₂)₂.

The conjugate of any one of the preceding clauses wherein the linker comprises NH(CH₂)₂O—(CH₂)₂—C(O)NH—(CH₂)₂NH, NH[(CH₂)₂O]₂—(CH₂)₂—C(O)NH—(CH₂)₂NH, NH[(CH₂)₂₀₁₆—(CH₂)₂—C(O)NH—(CH₂)₂NH, or NH[(CH₂)₂O]₁₂—(CH₂)₂—C(O)NH—(CH₂)₂NH.

The conjugate of any one of the preceding clauses wherein the linker comprises NH[(CH₂)₂O]ₙ—(CH₂)₂NH, where n is an integer from 1 to about 12.

The conjugate of any one of the preceding clauses wherein the linker comprises NH(CH₂)₂O—(CH₂)₂NH, NH[(CH₂)₂O]₂—(CH₂)₂NH, NH[(CH₂)₂₀₁₆—(CH₂)₂NH, or NH[(CH₂)₂O]₁₂—(CH₂)₂NH.

The conjugate of any one of the preceding clauses wherein the linker comprises NH[(CH₂)₂O]ₙ—(CH₂)₂NH—C(O)—(CH₂)₂—C(O), where n is an integer from 1 to about 12.

The conjugate of any one of the preceding clauses wherein the linker comprises NH(CH₂)₂O—(CH₂)₂NH—C(O)—(CH₂)₂—C(O), NH[(CH₂)₂O]₂—(CH₂)₂NH—C(O)—(CH₂)₂—C(O), NH[(CH₂)₂₀₁₆—(CH₂)₂NH—C(O)—(CH₂)₂—C(O), or NH[(CH₂)₂O]₁₂—(CH₂)₂NH—C(O)—(CH₂)₂—C(O).

The conjugate of any one of the preceding clauses comprising the formula

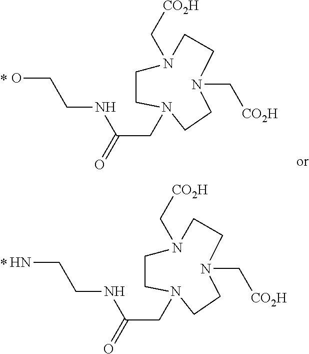 [SMILES: O=C(O)CN1CCN(CC(=O)O)CCN(CC(=O)NCCO)CC1 ; NCCNC(=O)CN1CCN(CC(=O)O)CCN(CC(=O)O)CC1]

or a derivative thereof comprising a chelated metal.

The conjugate of any one of the preceding clauses where P comprises the formula

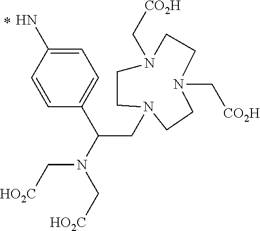 [SMILES: Nc1ccc(C(CN2CCN(CC(=O)O)CCN(CC(=O)O)CC2)N(CC(=O)O)CC(=O)O)cc1]

or a derivative thereof comprising a chelated metal.

The conjugate of any one of the preceding clauses comprising the formula

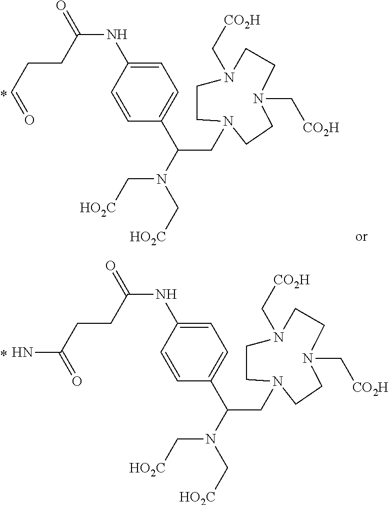 [SMILES: O=CCCC(=O)Nc1ccc(C(CN2CCN(CC(=O)O)CCN(CC(=O)O)CC2)N(CC(=O)O)CC(=O)O)cc1 ; NC(=O)CCC(=O)Nc1ccc(C(CN2CCN(CC(=O)O)CCN(CC(=O)O)CC2)N(CC(=O)O)CC(=O)O)cc1]

or a derivative thereof comprising a chelated metal.

The conjugate of any one of the preceding clauses wherein the targeting agent is a radical of a PSMA binding ligand or PSMA inhibitor.

The conjugate of any one of the preceding clauses wherein the targeting agent is a radical of a PSMA inhibitor.

The conjugate of any one of the preceding clauses comprising the formula

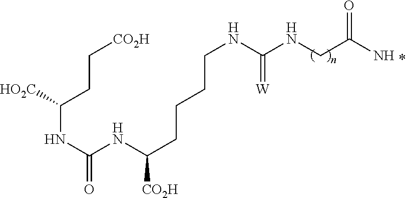

wherein n is an integer selected from 1, 2, 3, 4, 5, 6, 7, 8, 9, or 10; or

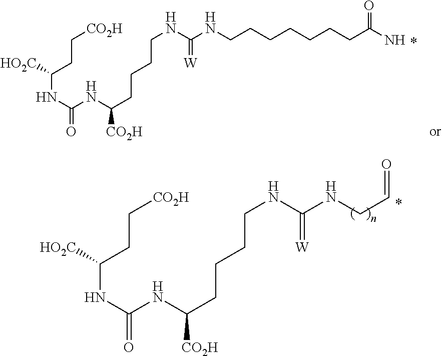

wherein n is an integer selected from 1, 2, 3, 4, 5, 6, 7, 8, 9, or 10; or

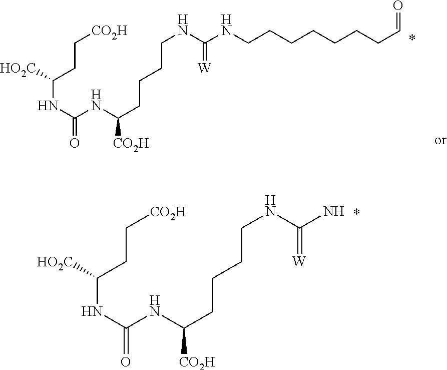

where W is O or S.

The conjugate of any one of the preceding clauses wherein the linker comprises a polypeptide.

The conjugate of any one of the preceding clauses wherein the linker comprises a polypeptide comprising phenylalanine, lysine, arginine, or aspartic acid, or a combination thereof.

The conjugate of any one of the preceding clauses wherein the linker comprises a lysine.

The conjugate of any one of the preceding clauses wherein the linker comprises Lys.

The conjugate of any one of the preceding clauses wherein the linker comprises Arg-Lys.

The conjugate of any one of the preceding clauses wherein the linker comprises Asp-Arg-Lys.

The conjugate of any one of the preceding clauses wherein the linker comprises Arg-Asp-Arg.

The conjugate of any one of the preceding clauses wherein the linker comprises Arg-Asp-Arg-Lys.

The conjugate of any one of the preceding clauses wherein the linker comprises Phe-Arg-Asp.

The conjugate of any one of the preceding clauses wherein the linker comprises Phe-Arg-Asp-Arg.

The conjugate of any one of the preceding clauses wherein the linker comprises Phe-Arg-Asp-Arg-Lys.

The conjugate of any one of the preceding clauses wherein the linker comprises Phe-Phe-Arg.

The conjugate of any one of the preceding clauses wherein the linker comprises Phe-Phe-Arg-Asp.

The conjugate of any one of the preceding clauses wherein the linker comprises Phe-Phe-Arg-Asp-Arg.

The conjugate of any one of the preceding clauses wherein the linker comprises Phe-Phe-Arg-Asp-Arg-Lys.

The conjugate of any one of the preceding clauses wherein the radical of the radionuclide or radionuclide containing group, or precursor thereof, or compound capable of binding a radionuclide or radionuclide containing group comprises a radical of NOTA. The conjugate of any one of the preceding clauses wherein P comprises the formula

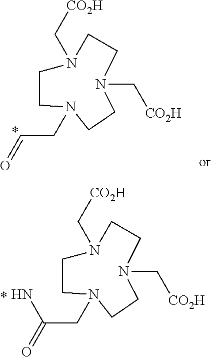

or a derivative thereof comprising a chelated metal.

The conjugate of any one of the preceding clauses comprising the formula

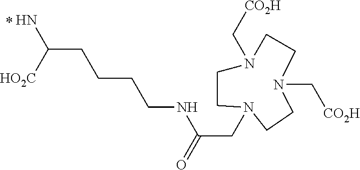 [SMILES: NC(CCCCNC(=O)CN1CCN(CC(=O)O)CCN(CC(=O)O)CC1)C(=O)O]

or a derivative thereof comprising a chelated metal.

The conjugate of any one of the preceding clauses comprising the formula

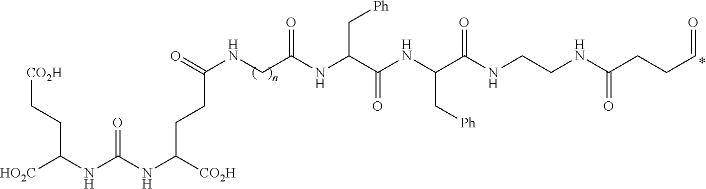

wherein n is an integer selected from 1, 2, 3, 4, 5, 6, 7, 8, 9, or 10.

The conjugate of any one of the preceding clauses comprising the formula

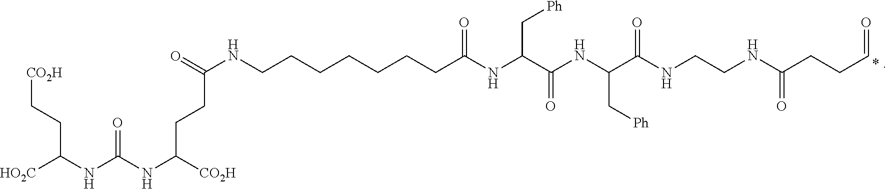 [SMILES: O=CCCC(=O)NCCNC(=O)C(Cc1ccccc1)NC(=O)C(Cc1ccccc1)NC(=O)CCCCCCCNC(=O)CCC(NC(=O)NC(CCC(=O)O)C(=O)O)C(=O)O]

The conjugate of any one of the preceding clauses wherein the linker comprises the formula

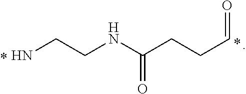

The conjugate of any one of the preceding clauses wherein the linker comprises the formula

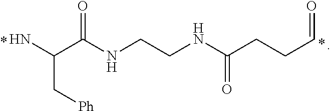 [SMILES: NC(Cc1ccccc1)C(=O)NCCNC(=O)CCC=O]

The conjugate of any one of the preceding clauses wherein the linker comprises the formula

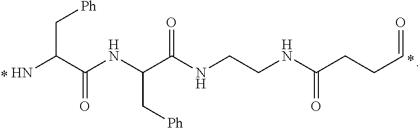 [SMILES: NC(Cc1ccccc1)C(=O)NC(Cc1ccccc1)C(=O)NCCNC(=O)CCC=O]

The conjugate of any one of the preceding clauses wherein one or more of the phenylalanines is L-phenylalanine.

The conjugate of any one of the preceding clauses comprising the formula

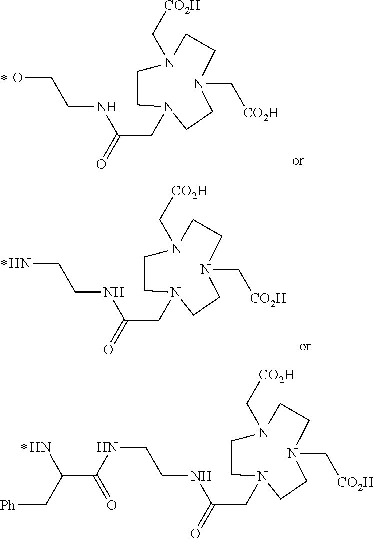 [SMILES: O=C(O)CN1CCN(CC(=O)O)CCN(CC(=O)NCCO)CC1 ; NCCNC(=O)CN1CCN(CC(=O)O)CCN(CC(=O)O)CC1 ; NC(Cc1ccccc1)C(=O)NCCNC(=O)CN1CCN(CC(=O)O)CCN(CC(=O)O)CC1]

or a derivative thereof comprising a chelated metal.

The conjugate of any one of the preceding clauses where P comprises the formula

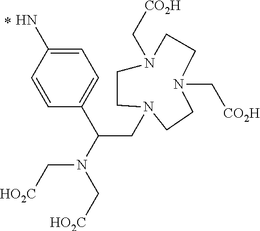 [SMILES: Nc1ccc(C(CN2CCN(CC(=O)O)CCN(CC(=O)O)CC2)N(CC(=O)O)CC(=O)O)cc1]

or a derivative thereof comprising a chelated metal.

The conjugate of any one of the preceding clauses comprising the formula

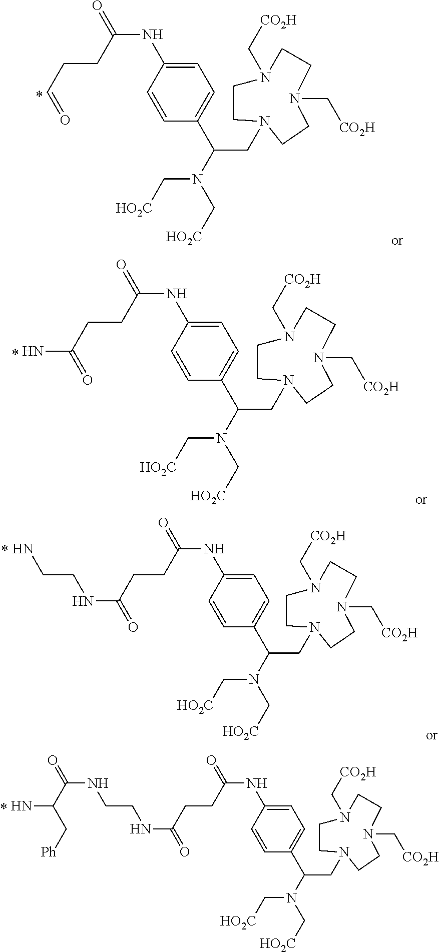 [SMILES: O=CCCC(=O)Nc1ccc(C(CN2CCN(CC(=O)O)CCN(CC(=O)O)CC2)N(CC(=O)O)CC(=O)O)cc1 ; NC(=O)CCC(=O)Nc1ccc(C(CN2CCN(CC(=O)O)CCN(CC(=O)O)CC2)N(CC(=O)O)CC(=O)O)cc1 ; NCCNC(=O)CCC(=O)Nc1ccc(C(CN2CCN(CC(=O)O)CCN(CC(=O)O)CC2)N(CC(=O)O)CC(=O)O)cc1 ; NC(Cc1ccccc1)C(=O)NCCNC(=O)CCC(=O)Nc1ccc(C(CN2CCN(CC(=O)O)CCN(CC(=O)O)CC2)N(CC(=O)O)CC(=O)O)cc1]

or a derivative thereof comprising a chelated metal.

The conjugate of any one of the preceding clauses wherein the radionuclide is a positron emitting radionuclide.

The conjugate of any one of the preceding clauses wherein the radionuclide is a metal ion.

The conjugate of any one of the preceding clauses wherein the radionuclide is a metal salt.

The conjugate of any one of the preceding clauses comprising an aluminum halide, such as an aluminum fluoride, aluminum chloride, aluminum bromide, or aluminum iodide.

The conjugate of any one of the preceding clauses comprising an aluminum fluoride.

The conjugate of any one of the preceding clauses comprising an aluminum ¹⁸F-fluoride.

The conjugate of any one of the preceding clauses comprising an aluminum iodide.

The conjugate of any one of the preceding clauses comprising an aluminum ¹²⁵I-iodide.

The conjugate of any one of the preceding clauses comprising a gallium ion.

The conjugate of any one of the preceding clauses comprising a ⁶⁶Ga ion.

The conjugate of any one of the preceding clauses comprising a ⁶⁸Ga ion.

The conjugate of any one of the preceding clauses comprising a zirconium ion.

The conjugate of any one of the preceding clauses comprising a ⁸⁹Zr ion.

The conjugate of any one of the preceding clauses comprising a copper ion.

The conjugate of any one of the preceding clauses comprising a ⁶⁴Cu ion.

The conjugate of any one of the preceding clauses wherein the radionuclide is a radiotherapy agent, such as iodine, including ¹³¹I, lutetium, including ¹⁷⁷Lu, yttrium, including ⁹⁰Y, strontium, including ⁸⁹Sr, samarium, including ¹⁵³Sm, and the like, or a radiotherapy agent containing group.

The conjugate of any one of the preceding clauses comprising a lutetium ion, such as a ¹⁷⁷Lu ion.

The conjugate of any one of the preceding clauses comprising a yttrium ion, such as a ⁹⁰Y ion.

A conjugate of the formulae

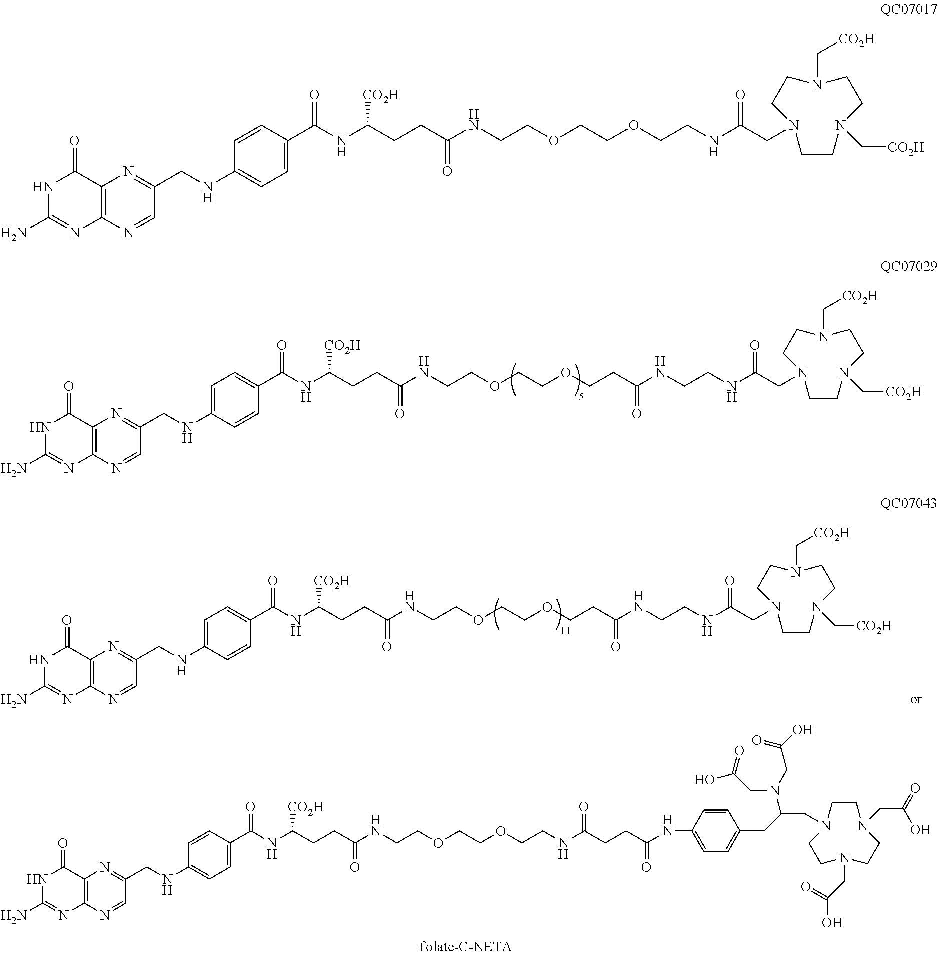

or a pharmaceutically acceptable salt thereof.

A conjugate of the formulae

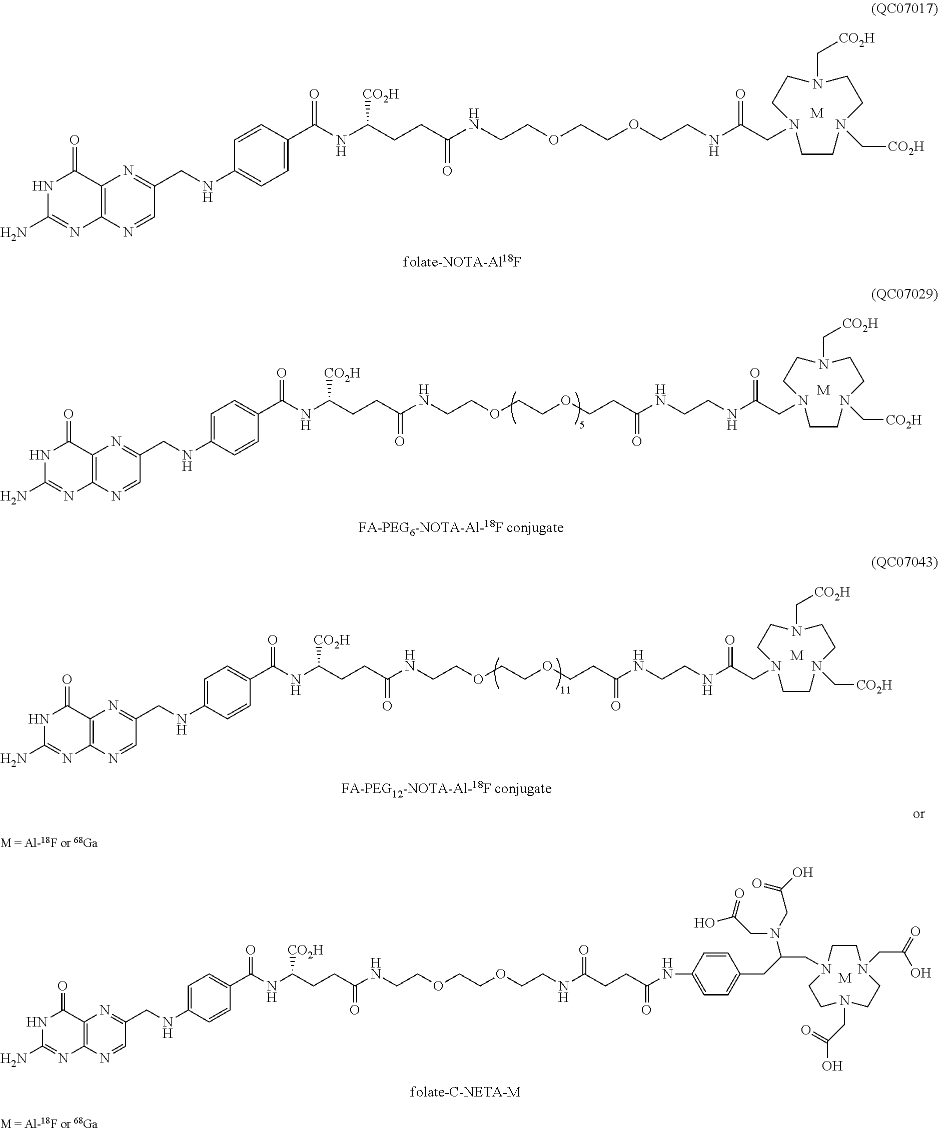

or a pharmaceutically acceptable salt thereof.

A conjugate of the formulae

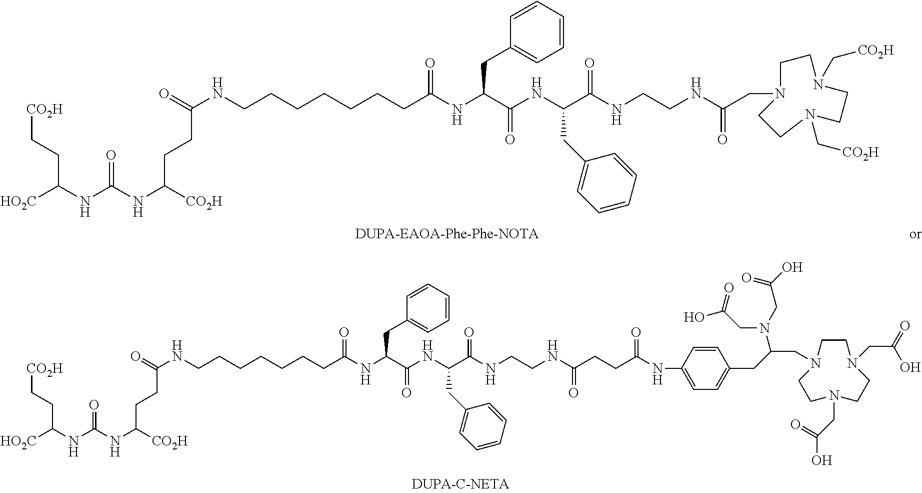 [SMILES: O=C(O)CCC(NC(=O)NC(CCC(=O)NCCCCCCCC(=O)N[C@@H](Cc1ccccc1)C(=O)N[C@@H](Cc1ccccc1)C(=O)NCCNC(=O)CN1CCN(CC(=O)O)CCN(CC(=O)O)CC1)C(=O)O)C(=O)O ; O=C(O)CCC(NC(=O)NC(CCC(=O)NCCCCCCCC(=O)N[C@@H](Cc1ccccc1)C(=O)N[C@@H](Cc1ccccc1)C(=O)NCCNC(=O)CCC(=O)Nc1ccc(CC(CN2CCN(CC(=O)O)CCN(CC(=O)O)CC2)N(CC(=O)O)CC(=O)O)cc1)C(=O)O)C(=O)O]

or a pharmaceutically acceptable salt thereof.

A conjugate of the formulae

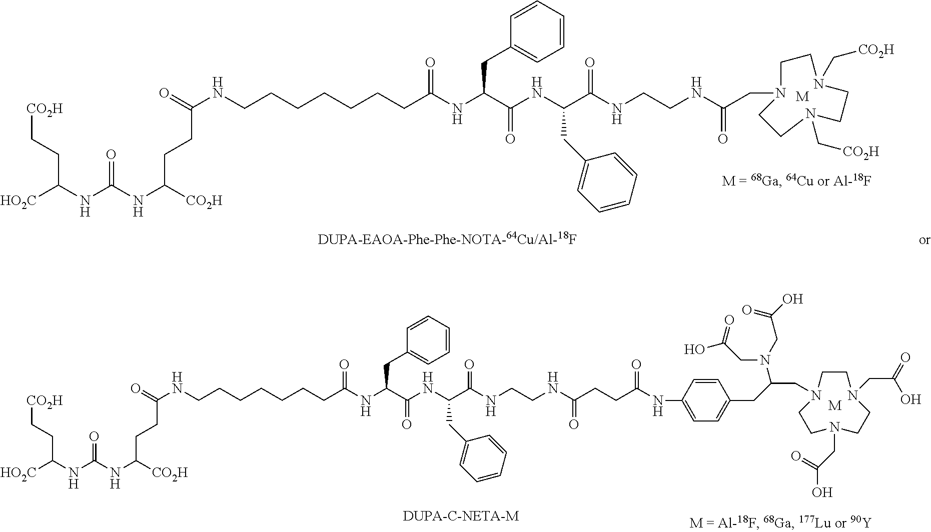

or a pharmaceutically acceptable salt thereof.

The conjugate of any one of the preceding clauses wherein P comprises the formula

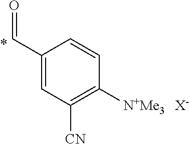

wherein X⁻is the conjugate base of an acid, such as trifluoromethanesulfonic acid.

The conjugate of any one of the preceding clauses comprising the formula

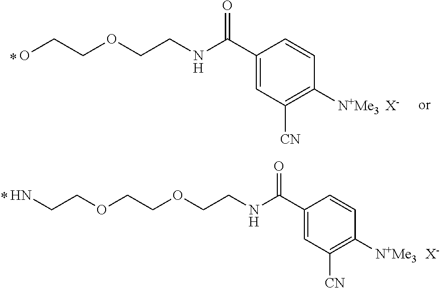

where X⁻is a conjugate base of an acid, such as trifluoromethanesulfonic acid.

The conjugate of any one of the preceding clauses wherein P comprises the formula

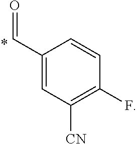 [SMILES: N#Cc1cc(C=O)ccc1F]

The conjugate of any one of the preceding clauses wherein P comprises the formula

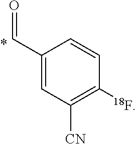

The conjugate of any one of the preceding clauses comprising the formula

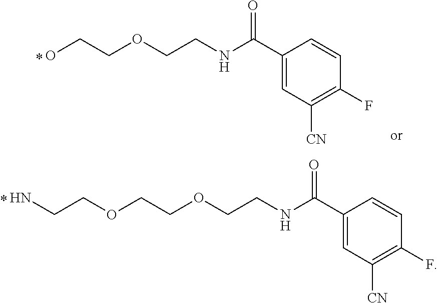

The conjugate of any one of the preceding clauses comprising the formula

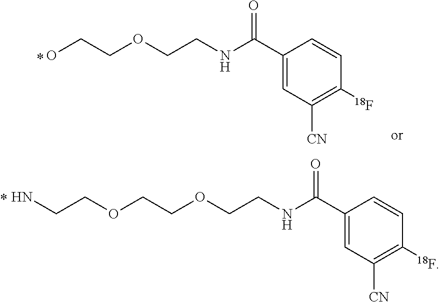

The conjugate of any one of the preceding clauses wherein P comprises the formula *NH—C(CH₂OH) 3.

The conjugate of any one of the preceding clauses comprising a boron fluoride. The conjugate of any one of the preceding clauses comprising a boron ¹⁸F-fluoride.

A pharmaceutical composition comprising one or more of the conjugates of any one of the preceding clauses, in combination with one or more carriers, diluents, or excipients, or a combination thereof.

A unit dose or unit dosage form composition comprising a diagnostically effective amount of one or more of the conjugates of any one of the preceding clauses, optionally in combination with one or more carriers, diluents, or excipients, or a combination thereof for diagnosing and/or monitoring a pathogenic cell population, such as a cancer or inflammatory disease.

A unit dose or unit dosage form composition comprising a therapeutically effective amount of one or more of the conjugates of any one of the preceding clauses, optionally in combination with one or more carriers, diluents, or excipients, or a combination thereof for treating a pathogenic cell population, such as a cancer or inflammatory disease.

A composition for diagnosing and/or monitoring a disease or disease state caused at least in part by a pathogenic cell population, such as a cancer or inflammatory disease, in a host animal, the composition comprising a diagnostically effective amount of one or more of the conjugates of any one of the preceding clauses; or a pharmaceutical composition comprising a diagnostically effective amount of one or more of the conjugates of any one of the preceding clauses, optionally further comprising one or more carriers, diluents, or excipients, or a combination thereof.

A composition for treating a disease or disease state caused at least in part by a pathogenic cell population, such as a cancer or inflammatory disease, in a host animal, the composition comprising a therapeutically effective amount of one or more of the conjugates of any one of the preceding clauses; or a pharmaceutical composition comprising a therapeutically effective amount of one or more of the conjugates of any one of the preceding clauses, optionally further comprising one or more carriers, diluents, or excipients, or a combination thereof.

A method for diagnosing and/or monitoring a disease or disease state caused at least in part by a pathogenic cell population, such as a cancer or inflammatory disease, in a host animal, the method comprising the step of administering to the host animal a diagnostically effective amount of one or more of the conjugates of any one of the preceding clauses; or a pharmaceutical composition comprising a diagnostically effective amount of one or more of the conjugates of any one of the preceding clauses, optionally further comprising one or more carriers, diluents, or excipients, or a combination thereof.

A method for treating a disease or disease state caused at least in part by a pathogenic cell population, such as a cancer or inflammatory disease, in a host animal, the method comprising the step of administering to the host animal a therapeutically effective amount of one or more of the conjugates of any one of the preceding clauses; or a pharmaceutical composition comprising a therapeutically effective amount of one or more of the conjugates of any one of the preceding clauses, optionally further comprising one or more carriers, diluents, or excipients, or a combination thereof.

Use of one or more of the conjugates of any one of the preceding clauses; or a pharmaceutical composition comprising one or more of the conjugates of any one of the preceding clauses, optionally further comprising one or more carriers, diluents, or excipients, or a combination thereof, in the manufacture of a medicament for diagnosing and/or monitoring a disease or disease state caused at least in part by a pathogenic cell population, such as a cancer or inflammatory disease, in a host animal.

Use of one or more of the conjugates of any one of the preceding clauses; or a pharmaceutical composition comprising one or more of the conjugates of any one of the preceding clauses, optionally further comprising one or more carriers, diluents, or excipients, or a combination thereof, in the manufacture of a medicament for treating a disease or disease state caused at least in part by a pathogenic cell population, such as a cancer or inflammatory disease, in a host animal.

A kit comprising one or more of the conjugates of any one of the preceding clauses, or a pharmaceutical composition thereof, optionally further comprising one or more carriers, diluents, or excipients, or a combination thereof; an optional solvent; an optional reaction container, and a set of instructions for preparing one or more radionuclides and combining the one or more radionuclides with the one or more of the conjugates to prepare an imaging agent, diagnostic agent, or therapeutic agent.

A kit comprising one or more of the conjugates of any one of the preceding clauses, or a pharmaceutical composition thereof, optionally further comprising one or more carriers, diluents, or excipients, or a combination thereof; an optional solvent; an optional reaction container, and a set of instructions for preparing one or more radionuclides and combining the one or more radionuclides with the one or more of the conjugates to prepare an imaging agent, diagnostic agent, or therapeutic agent.

It is to be understood that in each instance where a compound or chemical formula includes an atom or locus that is marked with or includes a (*), the (*) indicates that the compound or chemical formula is a radical having an open valence at that atom or locus, and that atom or locus is the location for attachment of another radical.

In another illustrative embodiment, the conjugate, composition, unit dose, method, use, or kit of any other embodiment described herein comprises a compound of formula

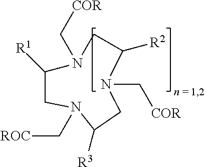

or a derivative thereof comprising a chelated metal; or a radical of the foregoing, where each R is in each instance independently selected to form a carboxylic acid or salt thereof, ester, or amide, and R¹, R², and R³, are each independently selected from hydrogen, and alkyl, cycloalkyl, aryl, arylalkyl, heteroaryl, and heteroarylalkyl, each of which is optionally substituted.

In another illustrative embodiment, the conjugate, composition, unit dose, method, use, or kit of any other embodiment described herein comprises 1,4,7,10-tetraazacyclododecane-1,4,7,10-tetraacetic acid (DOTA) or a derivative thereof comprising a chelated metal; or a radical of the foregoing.

In another illustrative embodiment, the conjugate, composition, unit dose, method, use, or kit of any other embodiment described herein comprises a compound of formula

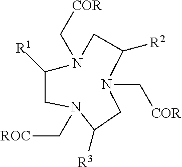

or a derivative thereof comprising a chelated metal; or a radical of the foregoing, where each R is in each instance independently selected to form a carboxylic acid or salt thereof, ester, or amide, and R¹, R², and R³, are each independently selected from hydrogen, and alkyl, cycloalkyl, aryl, arylalkyl, heteroaryl, and heteroarylalkyl, each of which is optionally substituted, such as the following illustrative compounds:

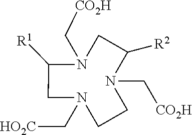

| R¹ | R² |
|---|---|
| 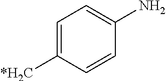 | H |
| 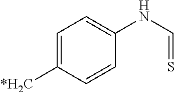 | H |
| 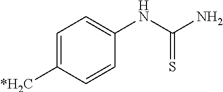 | H |
| 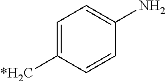 | 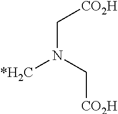 |
| 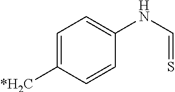 | 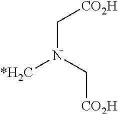 |
| 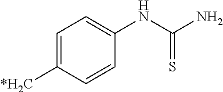 | 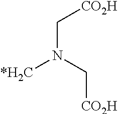 |
| 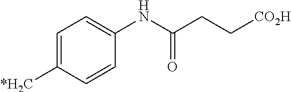 | 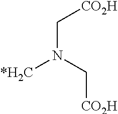 |
| 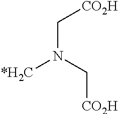 | 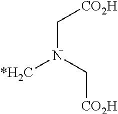 |
| 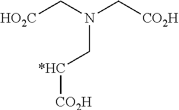 | 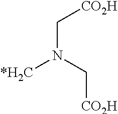 |

or a carboxylic acid salt or carboxamide derivative (CONH₂) thereof, or a radical of any of the foregoing; or a derivative thereof comprising a chelated metal.

In another illustrative embodiment, the conjugate, composition, unit dose, method, use, or kit of any other embodiment described herein comprises a compound of formula

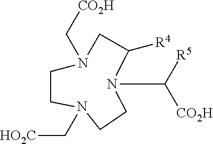

or a derivative thereof comprising a chelated metal; or a radical of the foregoing, where R⁴ and R⁵ are selected from hydrogen, and alkyl, cycloalkyl, aryl, arylalkyl, heteroaryl, and heteroarylalkyl, each of which is optionally substituted, such as the following illustrative compounds:

| R⁴ | R⁵ |
|---|---|
| 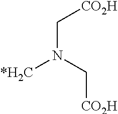 | 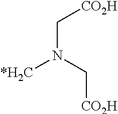 |
| 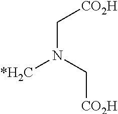 | 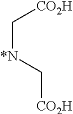 |

or a carboxylic acid salt or carboxamide derivative (CONH₂) thereof, or a radical of any of the foregoing; or a derivative thereof comprising a chelated metal.

In another illustrative embodiment, the conjugate, composition, unit dose, method, use, or kit of any other embodiment described herein comprises a compound of formula

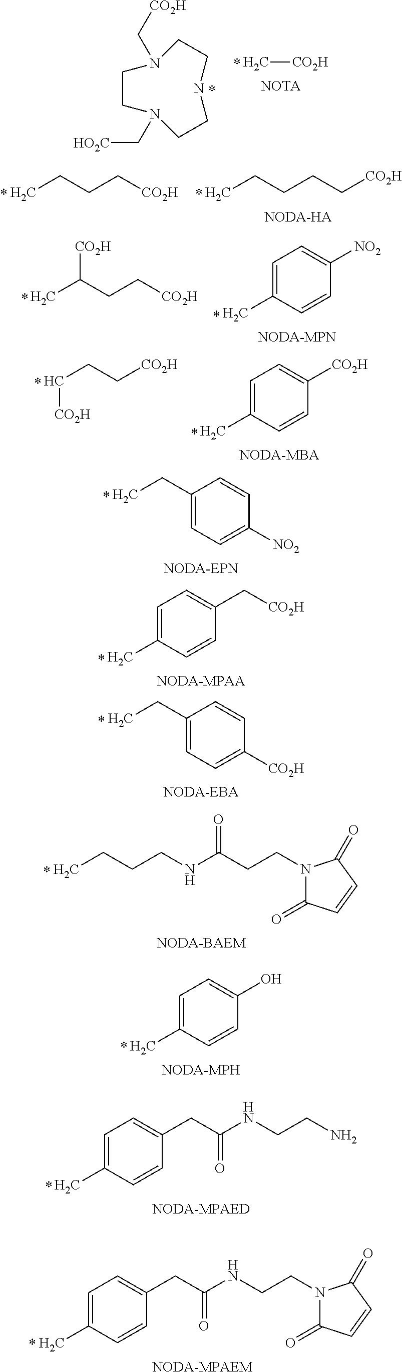

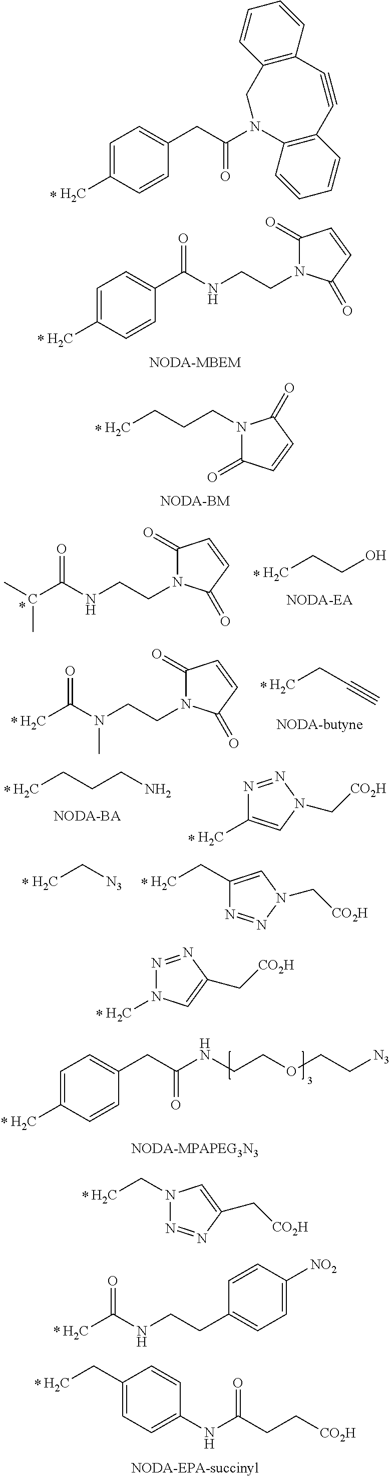

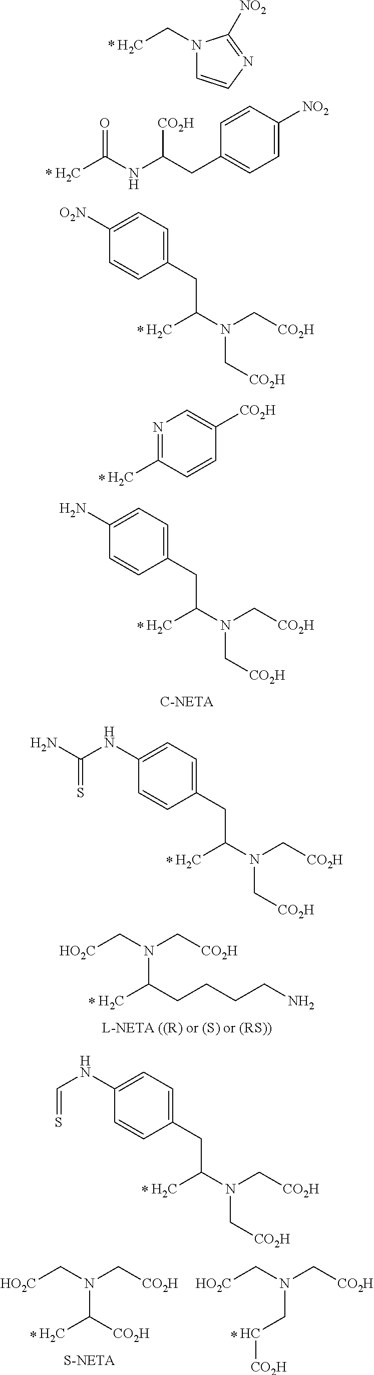

or a carboxylic acid salt or carboxamide derivative (CONH₂) thereof, or a radical of any of the foregoing; or a derivative thereof comprising a chelated metal.

In another illustrative embodiment, the conjugate, composition, unit dose, method, use, or kit of any other embodiment described herein comprises a compound selected from the formulae

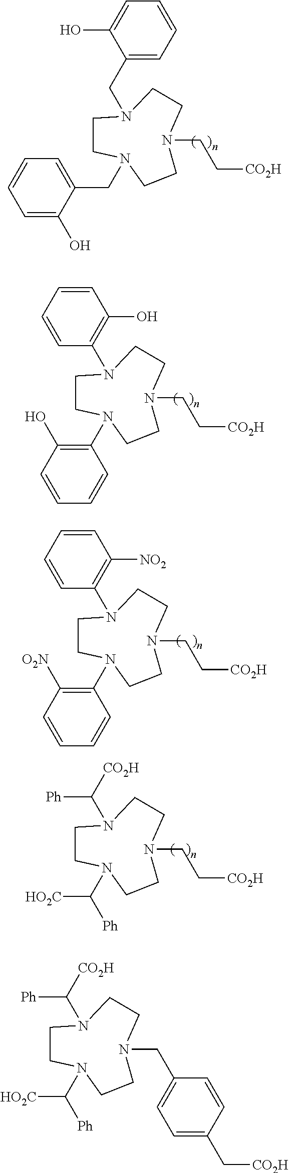

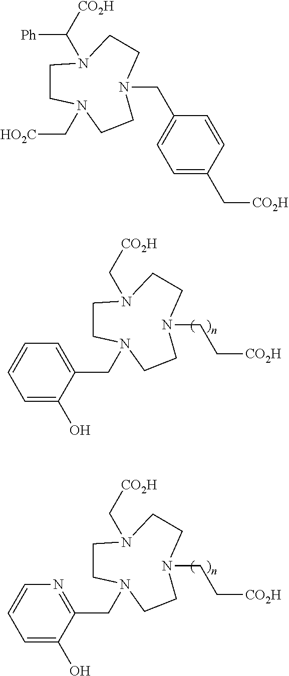

or a carboxylic acid salt or carboxamide derivative (CONH₂) thereof, or a radical of any of the foregoing, where n is an integer selected from 1, 2, 3, 4, 5, or 6; or a derivative thereof comprising a chelated metal.

As used herein the term “radical” generally refers to an open valence compound or chemical fragment that results after the removal of a hydrogen atom or a hydroxyl group from a carboxylic acid. For example, the following radicals may be formed from L-NETA

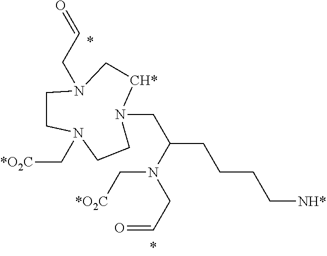

where each (*) atom is an open valence for attachment to a linker and/or targeting agent.

It is to be understood that the foregoing compounds and radicals thereof, may be further functionalized to attach reactive groups for the subsequent attachment of linkers and/or targeting groups. Illustratively, the following reactive intermediates are described herein

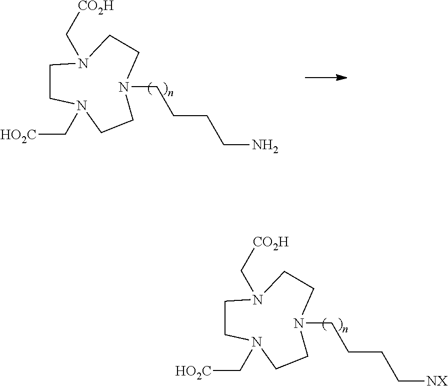

where n is 0 or 1, and NX is

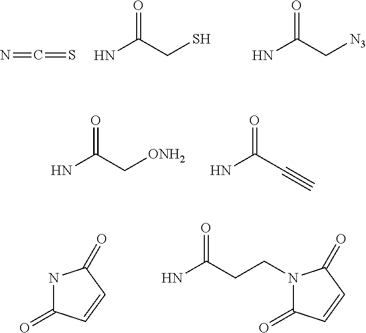

and the like.

It is to be understood that the following compounds, and metal chelates thereof, are not conjugates of the invention:

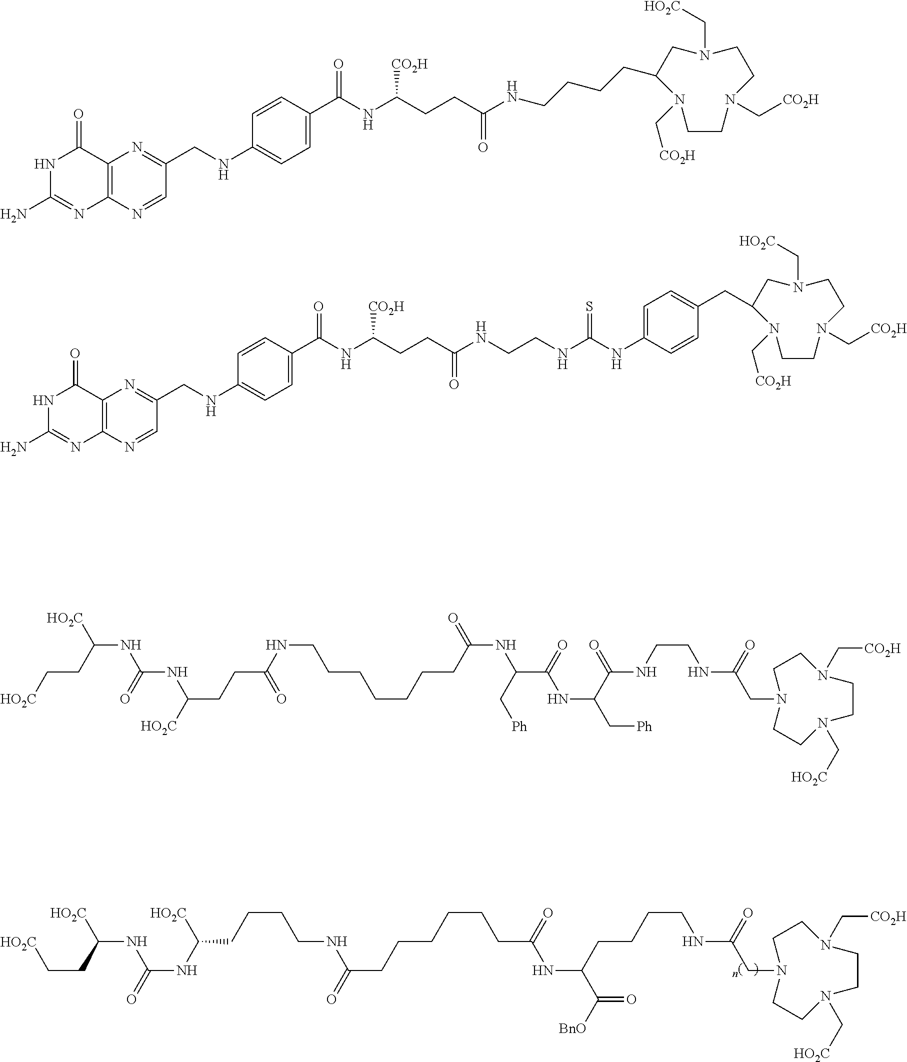

where n is 1 or 3.

The compounds described herein may contain one or more chiral centers, or may otherwise be capable of existing as multiple stereoisomers. It is to be understood that in one embodiment, the invention described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “alkyl” includes a chain of carbon atoms, which is optionally branched. As used herein, the terms “alkenyl” and “alkynyl” each include a chain of carbon atoms, which is optionally branched, and include at least one double bond or triple bond, respectively. It is to be understood that alkynyl may also include one or more double bonds. It is to be further understood that in certain embodiments, alkyl is advantageously of limited length, including C₁-C₂₄, C₁-C₁₂, C₁-C₈, C₁-C₆, and C₁-C₄. Illustratively, such particularly limited length alkyl groups, including C₁-C₈, C₁-C₆, and C₁-C₄ may be referred to as lower alkyl. It is to be further understood that in certain embodiments alkenyl and/or alkynyl may each be advantageously of limited length, including C₂-C₂₄, C₂-C₁₂, C₂-C₈, C₂-C₆, and C₂-C₄. Illustratively, such particularly limited length alkenyl and/or alkynyl groups, including C₂-C₈, C₂-C₆, and C₂-C₄ may be referred to as lower alkenyl and/or alkynyl. It is appreciated herein that shorter alkyl, alkenyl, and/or alkynyl groups may add less lipophilicity to the compound and accordingly will have different pharmacokinetic behavior. In embodiments of the invention described herein, it is to be understood, in each case, that the recitation of alkyl refers to alkyl as defined herein, and optionally lower alkyl. In embodiments of the invention described herein, it is to be understood, in each case, that the recitation of alkenyl refers to alkenyl as defined herein, and optionally lower alkenyl. In embodiments of the invention described herein, it is to be understood, in each case, that the recitation of alkynyl refers to alkynyl as defined herein, and optionally lower alkynyl. Illustrative alkyl, alkenyl, and alkynyl groups are, but not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like, and the corresponding groups containing one or more double and/or triple bonds, or a combination thereof.

As used herein, the term “alkylene” includes a divalent chain of carbon atoms, which is optionally branched. As used herein, the term “alkenylene” and “alkynylene” includes a divalent chain of carbon atoms, which is optionally branched, and includes at least one double bond or triple bond, respectively. It is to be understood that alkynylene may also include one or more double bonds. It is to be further understood that in certain embodiments, alkylene is advantageously of limited length, including C₁-C₂₄, C₁-C₁₂, C₁-C₈, C₁-C₆, and C₁-C₄. Illustratively, such particularly limited length alkylene groups, including C₁-C₈, C₁-C₆, and C₁-C₄ may be referred to as lower alkylene. It is to be further understood that in certain embodiments alkenylene and/or alkynylene may each be advantageously of limited length, including C₂-C₂₄, C₂-C₁₂, C₂-C₈, C₂-C₆, and C₂-C₄. Illustratively, such particularly limited length alkenylene and/or alkynylene groups, including C₂-C₈, C₂-C₆, and C₂-C₄ may be referred to as lower alkenylene and/or alkynylene. It is appreciated herein that shorter alkylene, alkenylene, and/or alkynylene groups may add less lipophilicity to the compound and accordingly will have different pharmacokinetic behavior. In embodiments of the invention described herein, it is to be understood, in each case, that the recitation of alkylene, alkenylene, and alkynylene refers to alkylene, alkenylene, and alkynylene as defined herein, and optionally lower alkylene, alkenylene, and alkynylene. Illustrative alkyl groups are, but not limited to, methylene, ethylene, n-propylene, isopropylene, n-butylene, isobutylene, sec-butylene, pentylene, 1,2-pentylene, 1,3-pentylene, hexylene, heptylene, octylene, and the like.

As used herein, the term “linker” includes is a chain of atoms that connects two or more functional parts of a molecule to form a conjugate. Illustratively, the chain of atoms is selected from C, N, O, S, Si, and P, or C, N, O, S, and P, or C, N, O, and S. The chain of atoms covalently connects different functional capabilities of the conjugate, such as targeting agents, drugs, diagnostic agents, imaging agents, and the like. The linker may have a wide variety of lengths, such as in the range from about 2 to about 100 atoms in the contiguous backbone. The atoms used in forming the linker may be combined in all chemically relevant ways, such as chains of carbon atoms forming alkylene, alkenylene, and alkynylene groups, and the like; chains of carbon and oxygen atoms forming ethers, polyoxyalkylene groups, or when combined with carbonyl groups forming esters and carbonates, and the like; chains of carbon and nitrogen atoms forming amines, imines, polyamines, hydrazines, hydrazones, or when combined with carbonyl groups forming amides, ureas, semicarbazides, carbazides, and the like; chains of carbon, nitrogen, and oxygen atoms forming alkoxyamines, alkoxylamines, or when combined with carbonyl groups forming urethanes, amino acids, acyloxylamines, hydroxamic acids, and the like; and many others. In addition, it is to be understood that the atoms forming the chain in each of the foregoing illustrative embodiments may be either saturated or unsaturated, thus forming single, double, or triple bonds, such that for example, alkanes, alkenes, alkynes, imines, and the like may be radicals that are included in the linker. In addition, it is to be understood that the atoms forming the linker may also be cyclized upon each other or be part of cyclic structure to form divalent cyclic structures that form the linker, including cyclo alkanes, cyclic ethers, cyclic amines, and other heterocycles, arylenes, heteroarylenes, and the like in the linker. In this latter arrangement, it is to be understood that the linker length may be defined by any pathway through the one or more cyclic structures. Illustratively, the linker length is defined by the shortest pathway through the each one of the cyclic structures. It is to be understood that the linkers may be optionally substituted at any one or more of the open valences along the chain of atoms, such as optional substituents on any of the carbon, nitrogen, silicon, or phosphorus atoms. It is also to be understood that the linker may connect the two or more functional parts of a molecule to form a conjugate at any open valence, and it is not necessary that any of the two or more functional parts of a molecule forming the conjugate are attached at any apparent end of the linker.

As used herein, the term “cycloalkyl” includes a chain of carbon atoms, which is optionally branched, where at least a portion of the chain in cyclic. It is to be understood that cycloalkylalkyl is a subset of cycloalkyl. It is to be understood that cycloalkyl may be polycyclic. Illustrative cycloalkyl include, but are not limited to, cyclopropyl, cyclopentyl, cyclohexyl, 2-methylcyclopropyl, cyclopentyleth-2-yl, adamantyl, and the like. As used herein, the term “cycloalkenyl” includes a chain of carbon atoms, which is optionally branched, and includes at least one double bond, where at least a portion of the chain in cyclic. It is to be understood that the one or more double bonds may be in the cyclic portion of cycloalkenyl and/or the non-cyclic portion of cycloalkenyl. It is to be understood that cycloalkenylalkyl and cycloalkylalkenyl are each subsets of cycloalkenyl. It is to be understood that cycloalkyl may be polycyclic. Illustrative cycloalkenyl include, but are not limited to, cyclopentenyl, cyclohexylethen-2-yl, cycloheptenylpropenyl, and the like. It is to be further understood that chain forming cycloalkyl and/or cycloalkenyl is advantageously of limited length, including C₃-C₂₄, C₃-C₁₂, C₃-C₈, C₃-C₆, and C₅-C₆. It is appreciated herein that shorter alkyl and/or alkenyl chains forming cycloalkyl and/or cycloalkenyl, respectively, may add less lipophilicity to the compound and accordingly will have different pharmacokinetic behavior.

As used herein, the term “heteroalkyl” includes a chain of atoms that includes both carbon and at least one heteroatom, and is optionally branched. Illustrative heteroatoms include nitrogen, oxygen, and sulfur. In certain variations, illustrative heteroatoms also include phosphorus, and selenium. As used herein, the term “cycloheteroalkyl” including heterocyclyl and heterocycle, includes a chain of atoms that includes both carbon and at least one heteroatom, such as heteroalkyl, and is optionally branched, where at least a portion of the chain is cyclic. Illustrative heteroatoms include nitrogen, oxygen, and sulfur. In certain variations, illustrative heteroatoms also include phosphorus, and selenium. Illustrative cycloheteroalkyl include, but are not limited to, tetrahydrofuryl, pyrrolidinyl, tetrahydropyranyl, piperidinyl, morpholinyl, piperazinyl, homopiperazinyl, quinuclidinyl, and the like.

As used herein, the term “aryl” includes monocyclic and polycyclic aromatic carbocyclic groups, each of which may be optionally substituted. Illustrative aromatic carbocyclic groups described herein include, but are not limited to, phenyl, naphthyl, and the like. As used herein, the term “heteroaryl” includes aromatic heterocyclic groups, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

As used herein, the terms “optionally substituted aryl” and “optionally substituted heteroaryl” include the replacement of hydrogen atoms with other functional groups on the aryl or heteroaryl that is optionally substituted. Such other functional groups, also referred to herein as aryl substituents, illustratively include, but are not limited to, amino, hydroxy, halo, thio, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxy, thio, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

Illustrative substituents include, but are not limited to, a radical-(CH₂)ₓZ^{X}, where x is an integer from 0-6 and Z^{X} is selected from halogen, hydroxy, alkanoyloxy, including C₁-C₆ alkanoyloxy, optionally substituted aroyloxy, alkyl, including C₁-C₆ alkyl, alkoxy, including C₁-C₆ alkoxy, cycloalkyl, including C₃-C₈ cycloalkyl, cycloalkoxy, including C₃-C₈ cycloalkoxy, alkenyl, including C₂-C₆ alkenyl, alkynyl, including C₂-C₆ alkynyl, haloalkyl, including C₁-C₆ haloalkyl, haloalkoxy, including C₁-C₆ haloalkoxy, halocycloalkyl, including C₃-C₈ halocycloalkyl, halocycloalkoxy, including C₃-C₈ halocycloalkoxy, amino, C₁-C₆ alkylamino, (C₁-C₆ alkyl)(C₁-C₆ alkyl)amino, alkylcarbonylamino, N—(C₁-C₆ alkyl)alkylcarbonylamino, aminoalkyl, C₁-C₆ alkylaminoalkyl, (C₁-C₆ alkyl)(C₁-C₆ alkyl)aminoalkyl, alkylcarbonylaminoalkyl, N—(C₁-C₆ alkyl)alkylcarbonylaminoalkyl, cyano, and nitro; or Z× is selected from —CO₂R⁴ and —CONR⁵R⁶, where R⁴, R⁵, and R⁶ are each independently selected in each occurrence from hydrogen, C₁-C₆ alkyl, aryl-C₁-C₆ alkyl, and heteroaryl-C₁-C₆ alkyl.

It is to be understood that in every instance disclosed herein, the recitation of a range of integers for any variable describes the recited range, every individual member in the range, and every possible subrange for that variable. For example, the recitation that n is an integer from 0 to 8, describes that range, the individual and selectable values of 0, 1, 2, 3, 4, 5, 6, 7, and 8, such as n is 0, or n is 1, or n is 2, etc. In addition, the recitation that n is an integer from 0 to 8 also describes each and every subrange, each of which may for the basis of a further embodiment, such as n is an integer from 1 to 8, from 1 to 7, from 1 to 6, from 2 to 8, from 2 to 7, from 1 to 3, from 2 to 4, etc.

As used herein, the term “composition” generally refers to any product comprising the specified ingredients in the specified amounts, as well as any product which results, directly or indirectly, from combinations of the specified ingredients in the specified amounts. It is to be understood that the compositions described herein may be prepared from isolated compounds described herein or from salts, solutions, hydrates, solvates, and other forms of the compounds described herein. It is appreciated that certain functional groups, such as the hydroxy, amino, and like groups form complexes and/or coordination compounds with water and/or various solvents, in the various physical forms of the compounds. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein.

Illustratively, compositions may include one or more carriers, diluents, and/or excipients. The compounds described herein, or compositions containing them, may be formulated in a diagnostically or therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21st ed., 2005)).

The term “diagnostically effective amount” as used herein, refers to that amount of active compound or pharmaceutical agent that elicits the biological or medicinal response in a tissue system, animal or human that is being sought by a researcher, veterinarian, medical doctor or other clinician, which includes diagnosis and/or monitoring of the symptoms of the disease or disorder being treated. Illustrative diagnostically effective amounts of the conjugate to be administered to the host animal include about 1 μg/kg to about 10 mg/kg, 1 ng/kg to about 10 mg/kg, or from about 10 μg/kg to about 1 mg/kg, or from about 100 μg/kg to about 500 μg/kg.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill. Illustrative therapeutically effective amounts of the conjugate to be administered to the host animal include about 1 μg/kg to about 10 mg/kg, 1 ng/kg to about 10 mg/kg, or from about 10 μg/kg to about 1 mg/kg, or from about 100 μg/kg to about 500 μg/kg. The term “administering” as used herein includes all means of introducing the compounds and compositions described herein to the host animal, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically-acceptable carriers, adjuvants, and/or vehicles.

As used herein, the term “amino acid” refers generally to beta, gamma, and longer amino acids, such as amino acids of the formula:

—N(R)—(CR′R″)_{q}—C(O)—

where R is hydrogen, alkyl, acyl, or a suitable nitrogen protecting group, R′ and R″ are hydrogen or a substituent, each of which is independently selected in each occurrence, and q is an integer such as 1, 2, 3, 4, or 5. Illustratively, R′ and/or R″ independently correspond to, but are not limited to, hydrogen or the side chains present on naturally occurring amino acids, such as methyl, benzyl, hydroxymethyl, thiomethyl, carboxyl, carboxylmethyl, guanidinopropyl, and the like, and derivatives and protected derivatives thereof. The above described formula includes all stereoisomeric variations. For example, the amino acid may be selected from alanine, aspartic acid, asparagine, cysteine, glutamic acid, phenylalanine, histidine, isoleucine, lysine, leucine, methionine, proline, glutamine, arginine, serine, threonine, valine, tryptophan, tyrosine, and ornithine, and the like.

It is to be understood that in every instance disclosed herein, the recitation of a range of integers for any variable describes the recited range, every individual member in the range, and every possible subrange for that variable. For example, the recitation that n is an integer from 0 to 8, describes that range, the individual and selectable values of 0, 1, 2, 3, 4, 5, 6, 7, and 8, such as n is 0, or nis 1, or n is 2, etc. In addition, the recitation that n is an integer from 0 to 8 also describes each and every subrange, each of which may for the basis of a further embodiment, such as n is an integer from 1 to 8, from 1 to 7, from 1 to 6, from 2 to 8, from 2 to 7, from 1 to 3, from 2 to 4, etc.

In another embodiment, the linkers described herein include a polyether, such as the linkers of the following formulae:

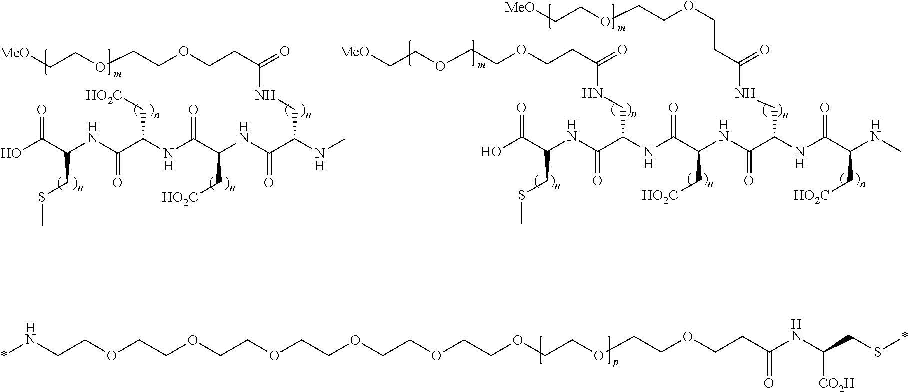

where m is an integer independently selected in each instance from 1 to about 8; p is an integer selected from 1 to about 10; and n is an integer independently selected in each instance from 1 to about 3. In one aspect, m is independently in each instance 1 to about 3. In another aspect, n is 1 in each instance. In another aspect, p is independently in each instance about 4 to about 6. Illustratively, the corresponding polypropylene polyethers corresponding to the foregoing are described herein and may be included in the conjugates as linkers. In addition, it is appreciated that mixed polyethylene and polypropylene polyethers may be included in the conjugates as linkers. Further, cyclic variations of the foregoing polyether compounds, such as those that include tetrahydrofuranyl, 1,3-dioxanes, 1,4-dioxanes, and the like are described herein.

In another embodiment, the linkers described herein include a plurality of hydroxyl functional groups, such as linkers that incorporate monosaccharides, oligosaccharides, polysaccharides, and the like. It is to be understood that the polyhydroxyl containing linkers comprise a plurality of —(CROH)-groups, where R is hydrogen or alkyl.

In another embodiment, the linkers include one or more of the following diradicals:

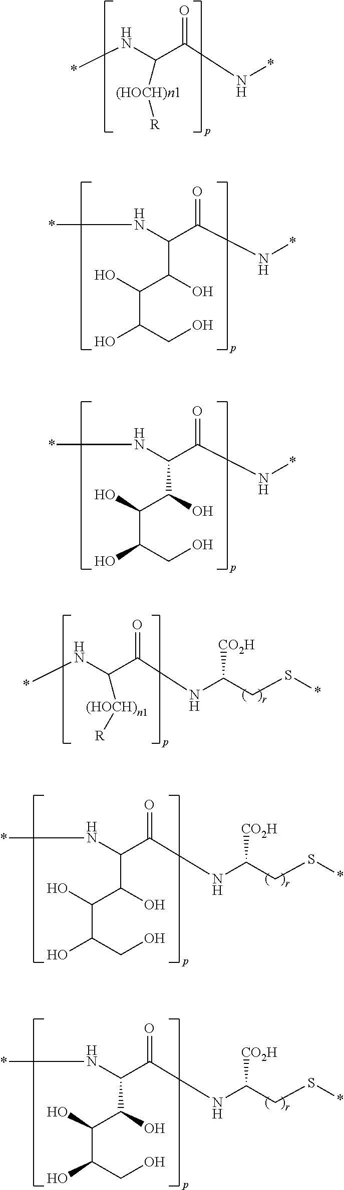

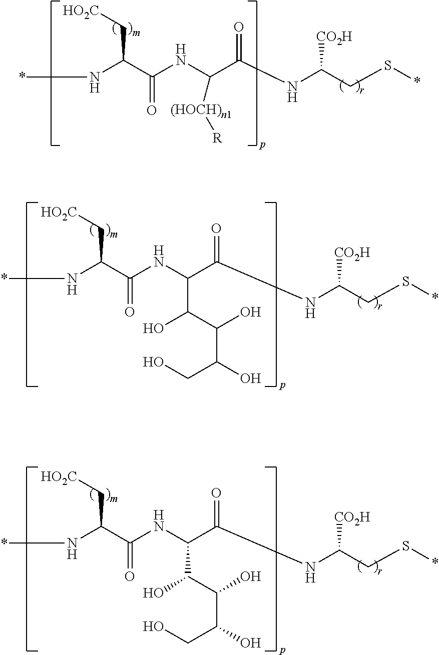

wherein R is H, alkyl, cycloalkyl, or arylalkyl; m is an integer from 1 to about 3; n1 is an integer from 1 to about 5, or n1 is an integer from 2 to about 5, p is an integer from 1 to about 5, and r is an integer selected from 1 to about 3. In one aspect, the integer n is 3 or 4. In another aspect, the integer p is 3 or 4. In another aspect, the integer r is 1.

In another embodiment, the linkers include one or more of the following diradicals:

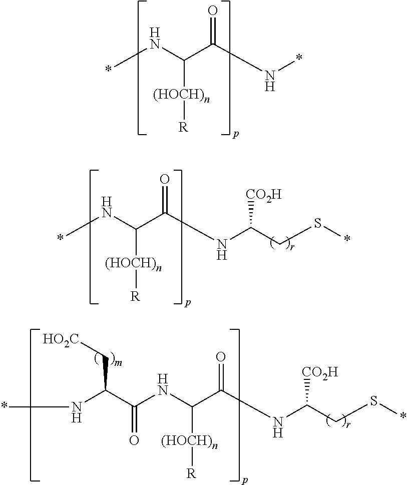

wherein R is H, alkyl, cycloalkyl, or arylalkyl; m is an integer from 1 to about 3; n is an integer from 1 to about 5, or from 2 to about 5, p is an integer from 1 to about 5, and r is an integer selected from 1 to about 3. In one aspect, the integer n is 3 or 4. In another aspect, the integer p is 3 or 4. In another aspect, the integer r is 1.

In another embodiment, the linker includes one or more of the following cyclic polyhydroxyl groups:

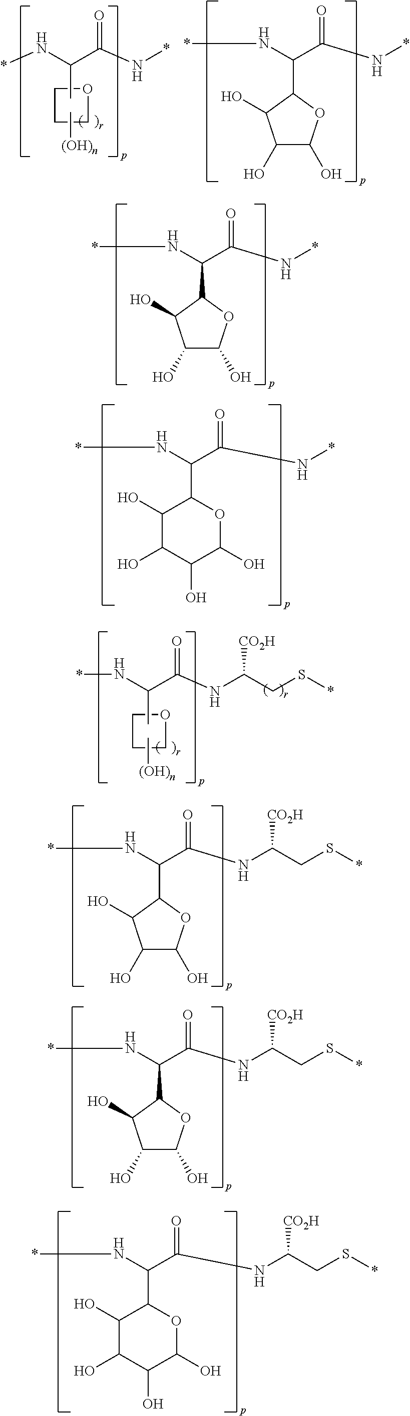

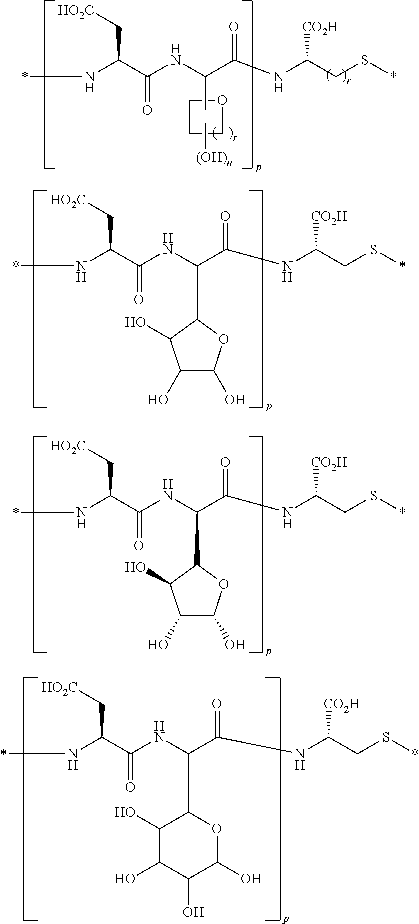

wherein n is an integer from 2 to about 5, p is an integer from 1 to about 5, and each r is an independently selected integer from 1 to about 4. In one aspect, the integer n is 3 or 4. In another aspect, the integer p is 3 or 4. In another aspect, each integer r is independently 2 or 3. It is understood that all stereochemical forms of such sections of the linkers are described herein. For example, in the above formula, the section may be derived from ribose, xylose, glucose, mannose, galactose, or other sugar and retain the stereochemical arrangements of pendant hydroxyl and alkyl groups present on those molecules. In addition, it is to be understood that in the foregoing formulae, various deoxy compounds are also described. Illustratively, compounds of the following formulae are described:

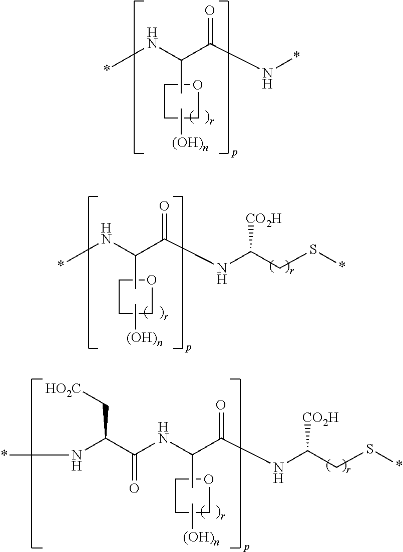

wherein n is equal to or less than r, such as when r is 2 or 3, n is 1 or 2, or 1, 2, or 3, respectively.

In another embodiment, the linker includes a polyhydroxyl compound of the following formula:

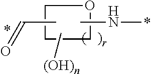

wherein n and r are each an integer selected from 1 to about 3. In one aspect, the linker includes one or more polyhydroxyl compounds of the following formulae:

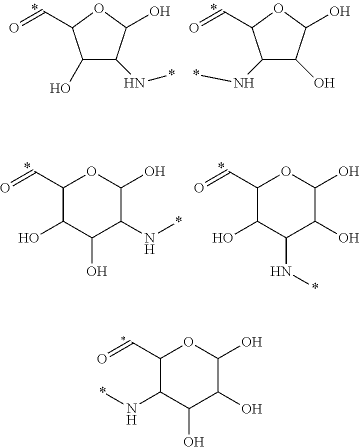

It is understood that all stereochemical forms of such sections of the linkers are described herein. For example, in the above formula, the section may be derived from ribose, xylose, glucose, mannose, galactose, or other sugar and retain the stereochemical arrangements of pendant hydroxyl and alkyl groups present on those molecules.

In another configuration, the linkers L described herein include polyhydroxyl groups that are spaced away from the backbone of the linker. In one embodiment, such carbohydrate groups or polyhydroxyl groups are connected to the back bone by a triazole group, forming triazole-linked linkers. Illustratively, such linkers include diradicals of the following formulae:

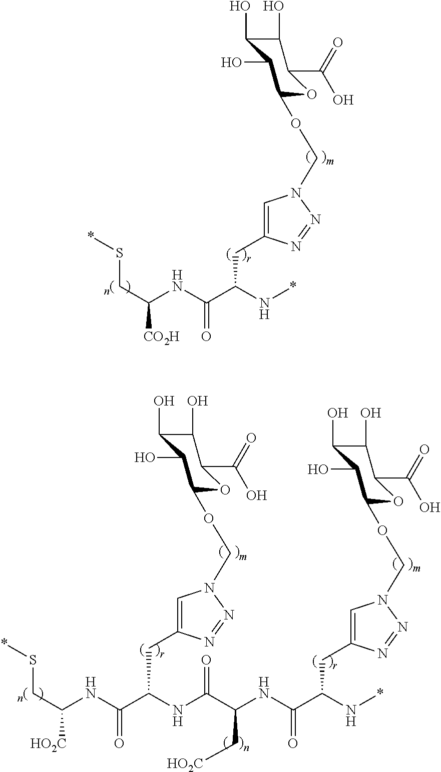

wherein n, m, and r are integers and are each independently selected in each instance from 1 to about 5. In one illustrative aspect, m is independently 2 or 3 in each instance. In another aspect, r is 1 in each instance. In another aspect, n is 1 in each instance. In one variation, the group connecting the polyhydroxyl group to the backbone of the linker is a different heteroaryl group, including but not limited to, pyrrole, pyrazole, 1,2,4-triazole, furan, oxazole, isoxazole, thienyl, thiazole, isothiazole, oxadiazole, and the like. Similarly, divalent 6-membered ring heteroaryl groups are described. Other variations of the foregoing illustrative linkers include oxyalkylene groups, such as the following formulae:

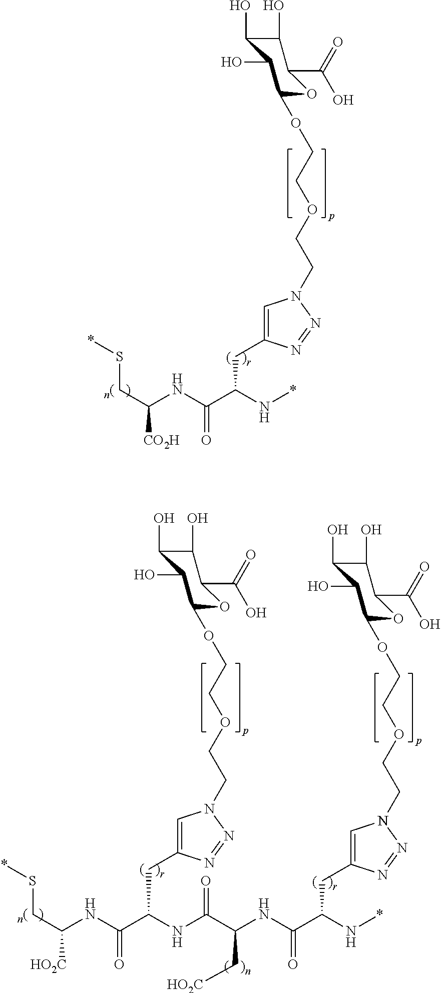

wherein n and r are integers and are each independently selected in each instance from 1 to about 5; and p is an integer selected from 1 to about 4.

In another embodiment, such carbohydrate groups or polyhydroxyl groups are connected to the back bone by an amide group, forming amide-linked linkers. Illustratively, such linkers include diradicals of the following formulae:

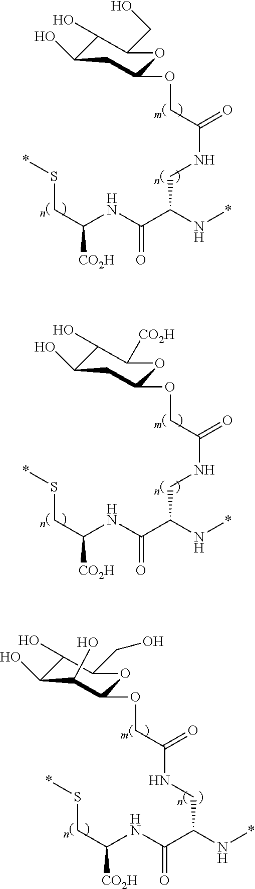

wherein each n is an independently selected integer from 1 to about 3, and m is an independently selected integer from 1 to about 22. In one illustrative aspect, each n is independently 1 or 2. In another illustrative aspect, m is selected from about 6 to about 10, illustratively 8. In one variation, the group connecting the polyhydroxyl group to the backbone of the linker is a different functional group, including but not limited to, esters, ureas, carbamates, acylhydrazones, and the like. Similarly, cyclic variations are described. Other variations of the foregoing illustrative linkers include oxyalkylene groups, such as the following formulae:

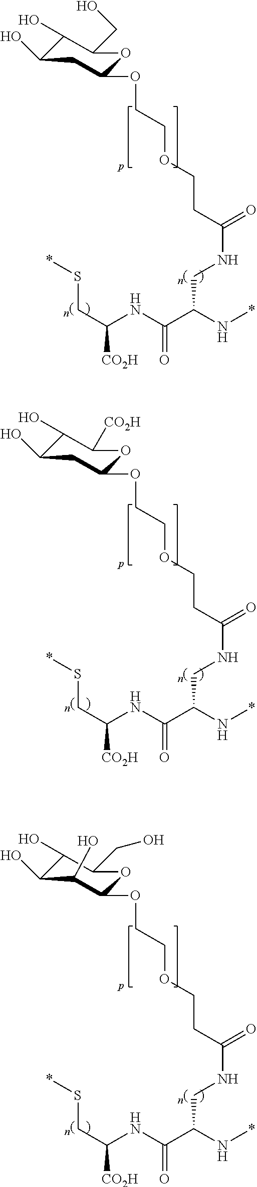

wherein n is in each instance an independently selected integer from 1 to about 5; and p is an integer selected from 1 to about 4.

In another embodiment, the linkers include one or more of the following diradicals:

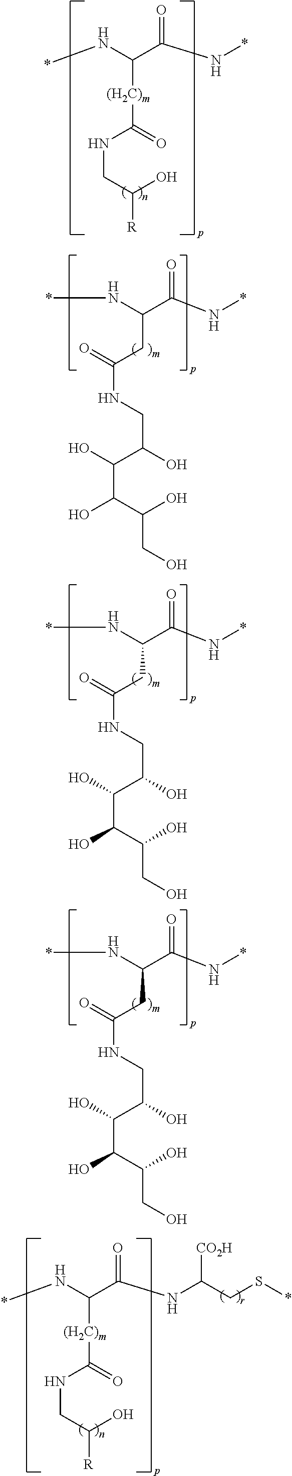

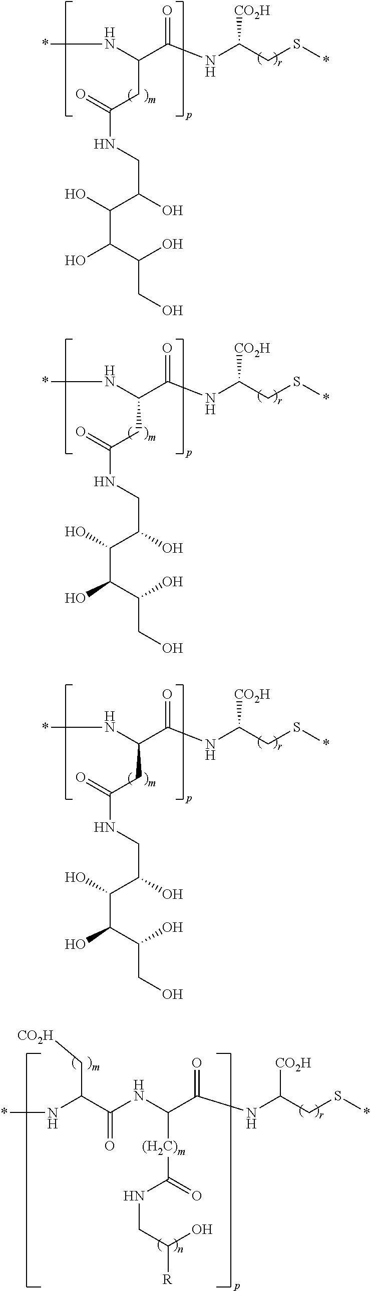

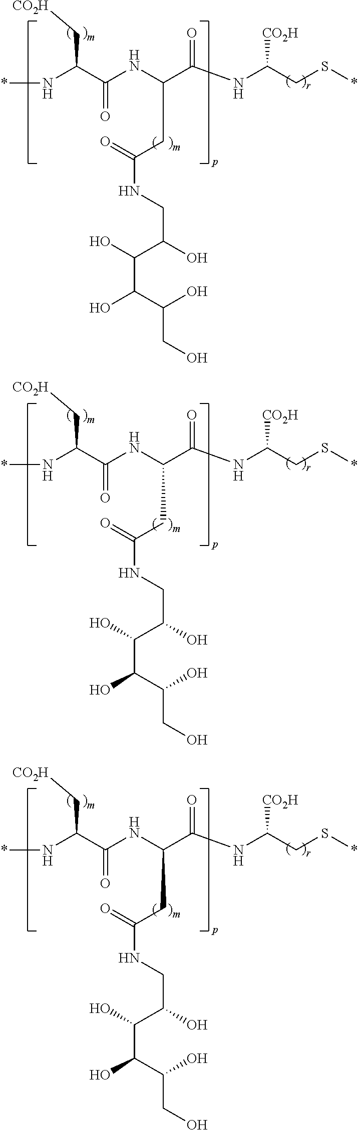

wherein R is H, alkyl, cycloalkyl, or arylalkyl; each m is an independently selected integer from 1 to about 3; each n is an independently selected integer from 1 to about 6, p is an integer from 1 to about 5, and r is an integer selected from 1 to about 3. In one variation, each n is independently 3 or 4. In another variation, the integer p is 3 or 4. In another variation, the integer r is 1.

In another embodiment, the linkers include one or more of the following diradicals:

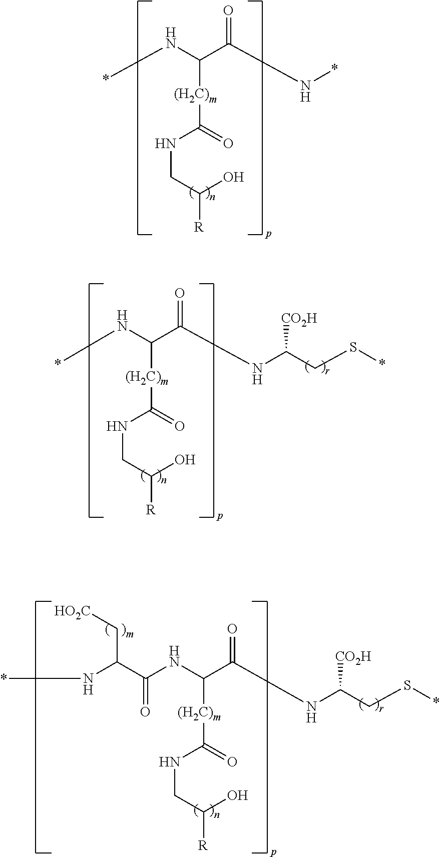

wherein R is H, alkyl, cycloalkyl, or arylalkyl; each m is an independently selected integer from 1 to about 3; each n is an independently selected integer from 2 to about 6, p is an integer from 1 to about 5, and r is an integer selected from 1 to about 3. In one variation, each n is independently 3 or 4. In another variation, the integer p is 3 or 4. In another variation, the integer r is 1.

In another embodiment, the linkers include one or more of the following diradicals:

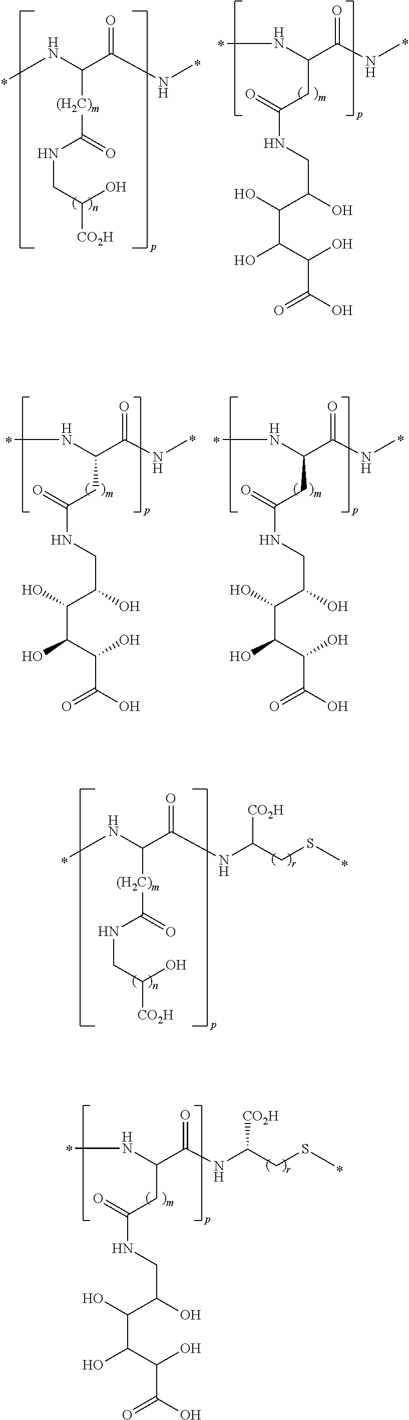

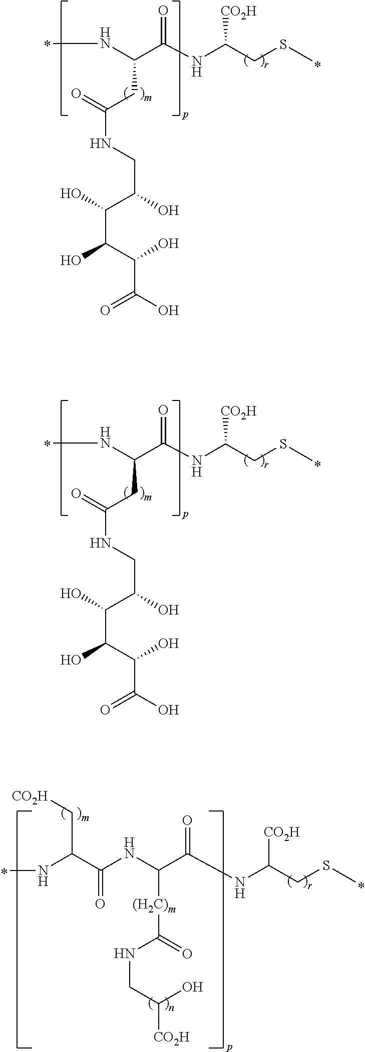

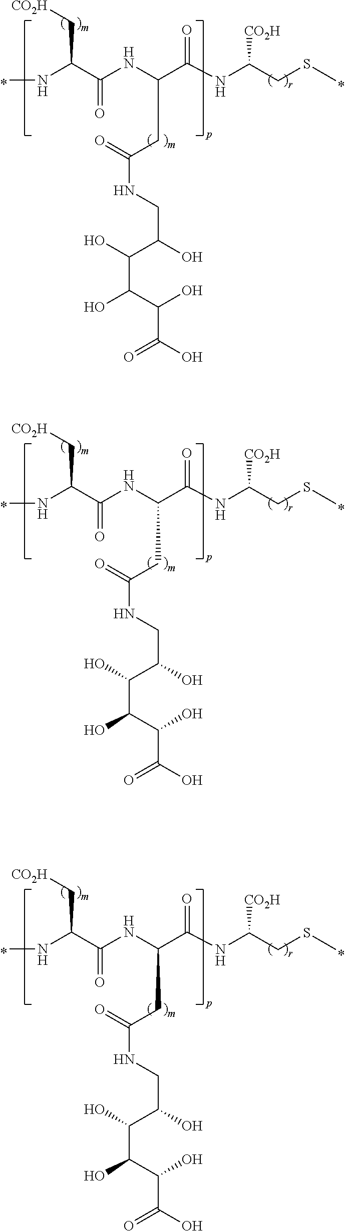

wherein each m is an independently selected integer from 1 to about 3; each n is an independently selected integer from 1 to about 6, p is an integer from 1 to about 5, and r is an integer selected from 1 to about 3. In one variation, each n is independently 3 or 4. In another variation, the integer p is 3 or 4. In another variation, the integer r is 1.

In another embodiment, the linkers include one or more of the following diradicals:

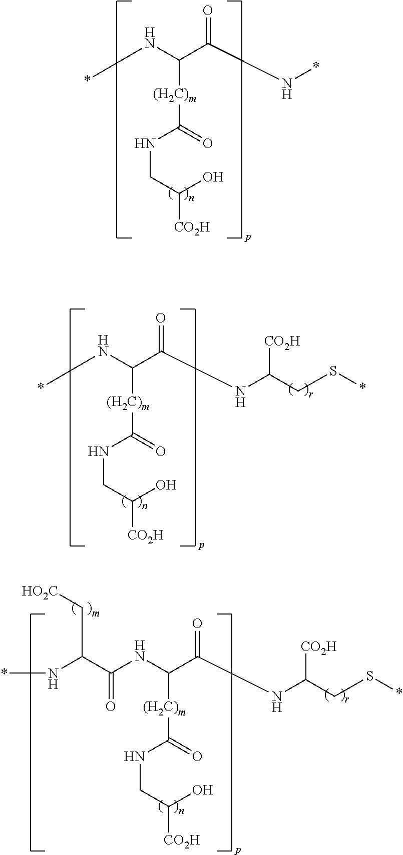

wherein each m is an independently selected integer from 1 to about 3; each n is an independently selected integer from 2 to about 6, p is an integer from 1 to about 5, and r is an integer selected from 1 to about 3. In one variation, each n is independently 3 or 4. In another variation, the integer p is 3 or 4. In another variation, the integer r is 1.

In another embodiment, the linkers include one or more of the following diradicals:

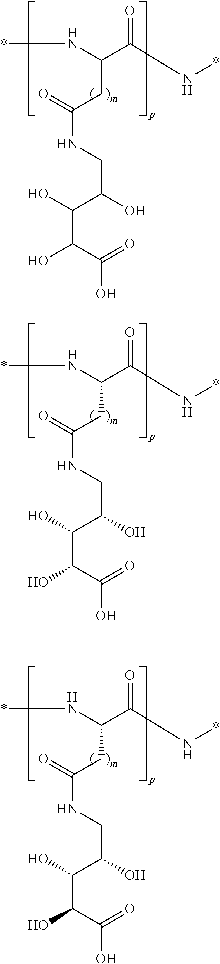

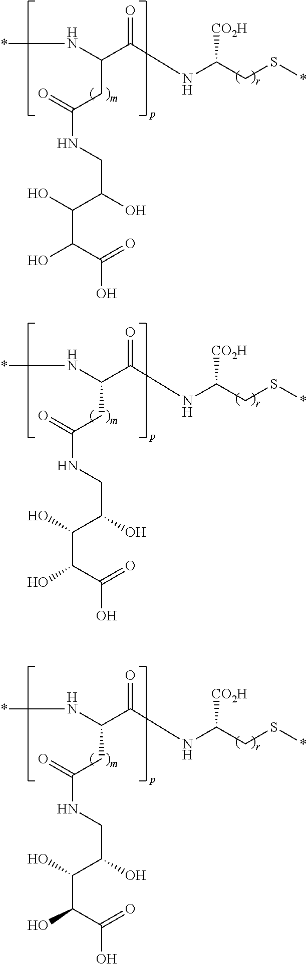

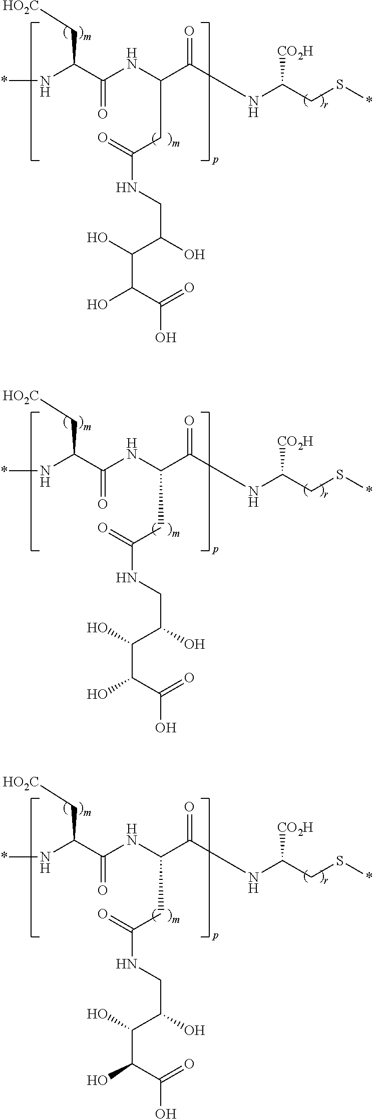

wherein each m is an independently selected integer from 1 to about 3, p is an integer from 1 to about 5, and r is an integer selected from 1 to about 3. In another variation, the integer p is 3 or 4. In another variation, the integer r is 1.

In another embodiment, the linker is a combination of backbone and branching side motifs such as is illustrated by the following formulae

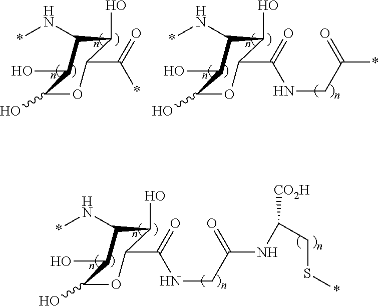

wherein n is an integer independently selected in each instance from 0 to about 3. The above formula are intended to represent 4, 5, 6, and even larger membered cyclic sugars. In addition, it is to be understood that the above formula may be modified to represent deoxy sugars, where one or more of the hydroxy groups present on the formulae are replaced by hydrogen, alkyl, or amino. In addition, it is to be understood that the corresponding carbonyl compounds are described by the above formulae, where one or more of the hydroxyl groups is oxidized to the corresponding carbonyl. In addition, in this illustrative embodiment, the pyranose includes both carboxyl and amino functional groups and (a) can be inserted into the backbone and (b) can provide synthetic handles for branching side chains in variations of this embodiment. Any of the pendant hydroxyl groups may be used to attach other chemical radicals, including additional sugars to prepare the corresponding oligosaccharides. Other variations of this embodiment are also described, including inserting the pyranose or other sugar into the backbone at a single carbon, i.e. a spiro arrangement, at a geminal pair of carbons, and like arrangements. For example, one or two ends of the linker, or the agent P, or the ligand B may be connected to the sugar to be inserted into the backbone in a 1,1; 1,2; 1,3; 1,4; 2,3, or other arrangement.

In another embodiment, the linkers include one or more amino groups of the following formulae:

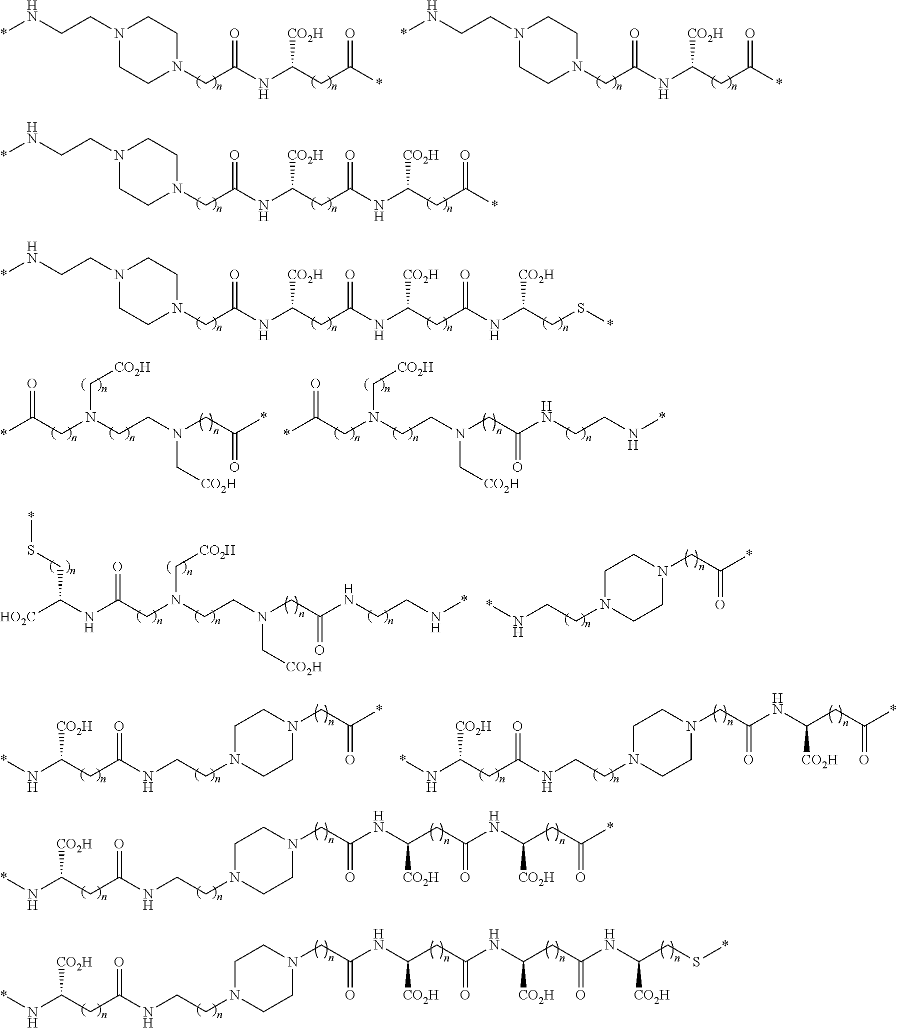

where each n is an integer independently selected in each instance from 1 to about 3. In one aspect, the each n is independently 1 or 2 in each instance. In another aspect, the integer n is 1 in each instance.

In another embodiment, the linker is a sulfuric acid ester, such as an alkyl ester of sulfuric acid. Illustratively, the linker is of the following formula:

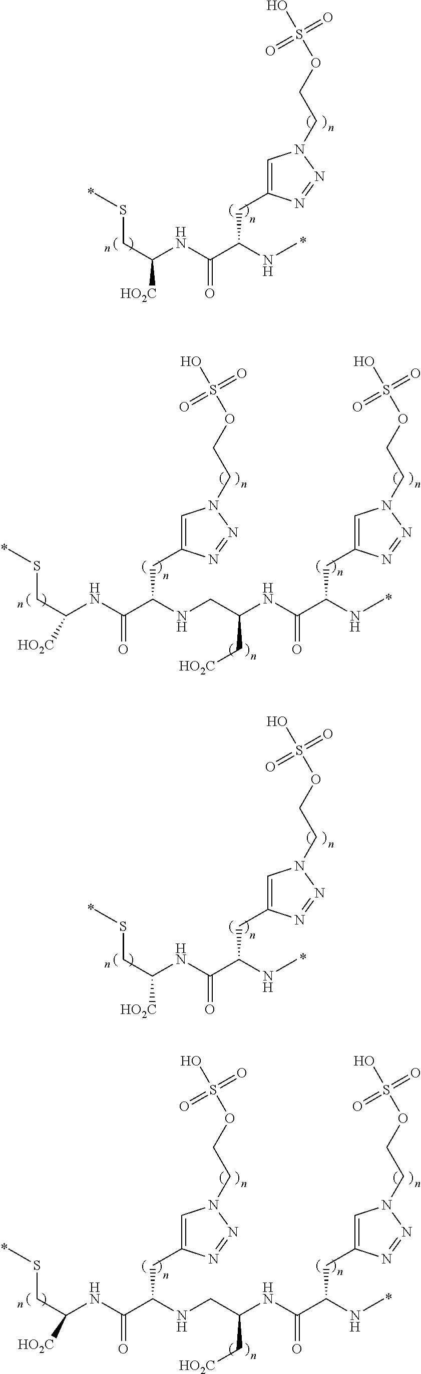

where each n is an integer independently selected in each instance from 1 to about 3. Illustratively, each n is independently 1 or 2 in each instance.

It is understood, that in such polyhydroxyl, polyamino, carboxylic acid, sulfuric acid, and like linkers that include free hydrogens bound to heteroatoms, one or more of those free hydrogen atoms may be protected with the appropriate hydroxyl, amino, or acid protecting group, respectively, or alternatively may be blocked as the corresponding pro-drugs, the latter of which are selected for the particular use, such as pro-drugs that release the parent drug under general or specific physiological conditions.

It is to be understood that in each of the foregoing illustrative examples, the stereochemical configurations shown herein are merely illustrative, and other stereochemical configurations are described. For example in one variation, the corresponding unnatural amino acid configurations may be included in the conjugated described herein as follows:

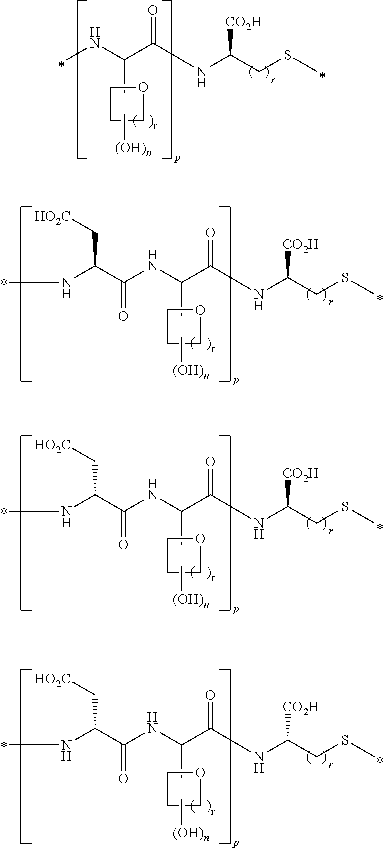

wherein each n is an independently selected integer from 2 to about 5, p is an integer from 1 to about 5, and r is an integer from 1 to about 4, as described above.

It is to be further understood that in the foregoing embodiments, open positions, such as (*) atoms are locations for attachment of the targeting agent B or the agent (P). In addition, it is to be understood that such attachment of either or both of B and A may be direct or through an intervening linker. Illustrative additional linkers are described in U.S. Pat. No. 7,601,332, the disclosure of which is incorporated herein by reference.

Illustrative bivalent radicals forming part of the linker.

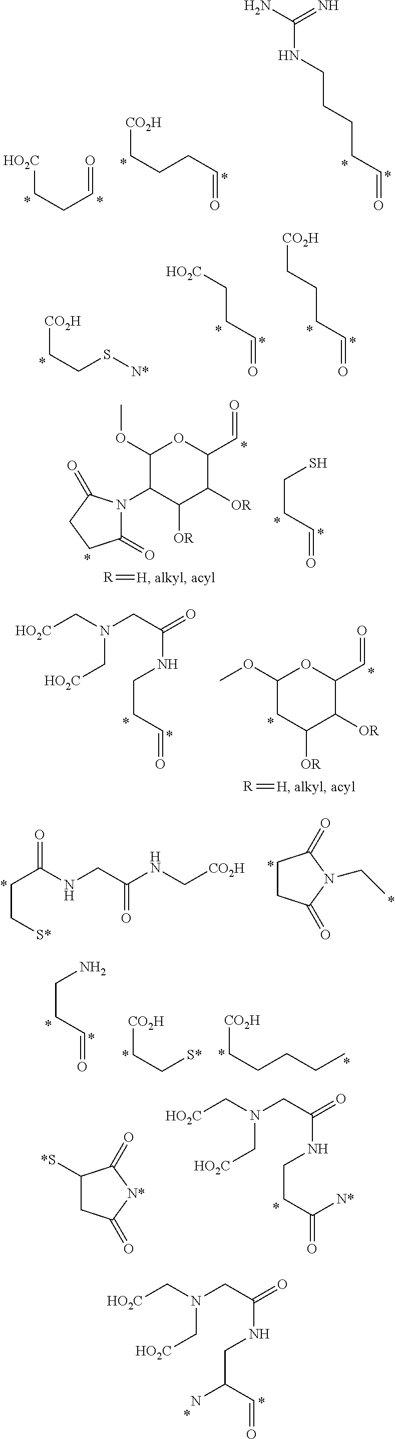

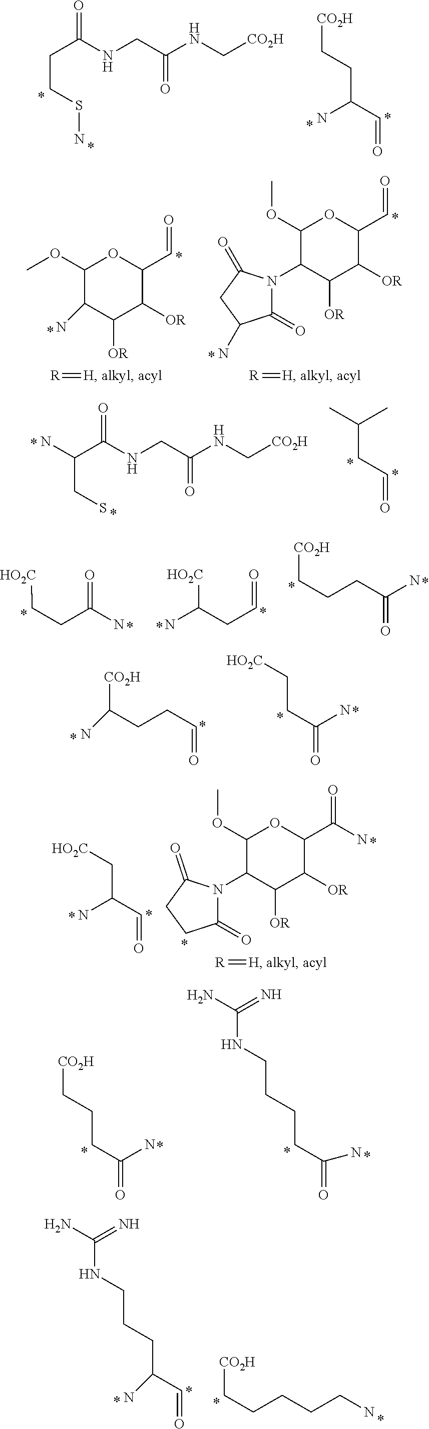

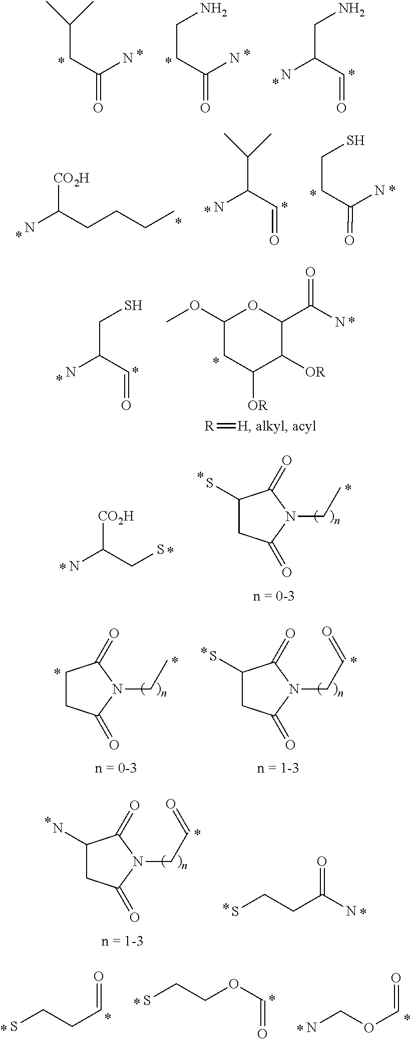

It is to be understood that the bivalent linkers may be combined in any chemically relevant way, either directly or via an intervening heteroatom to construct the linkers described herein.

In another embodiment, the polyvalent linkers described herein comprise a linker selected from the group consisting of carbonyl, thionocarbonyl, alkylene, cycloalkylene, alkylenecycloalkyl, alkylenecarbonyl, cycloalkylenecarbonyl, carbonylalkylcarbonyl, 1 alkylenesuccinimid-3-yl, 1 (carbonylalkyl) succinimid-3-yl, alkylenesulfoxyl, sulfonylalkyl, alkylenesulfoxylalkyl, alkylenesulfonylalkyl, carbonyltetrahydro-2H-pyranyl, carbonyltetrahydrofuranyl, 1-(carbonyltetrahydro-2H-pyranyl) succinimid-3-yl, and 1-(carbonyltetrahydrofuranyl) succinimid-3-yl.

In another embodiment, the compounds described herein comprise one or more amino acids.

The compounds described herein can be used for both human clinical medicine and veterinary applications. Thus, the host animal harboring the population of pathogenic cells and administered the compounds described herein can be human or, in the case of veterinary applications, can be a laboratory, agricultural, domestic, or wild animal. The present invention can be applied to host animals including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

The compounds, compositions, methods, and uses described herein are useful for diagnosing and/or monitoring diseases caused at least in part by populations of pathogenic cells, which may cause a variety of pathologies in host animals. As used herein, the term “pathogenic cells” or “population of pathogenic cells” generally refers to cancer cells, infectious agents such as bacteria and viruses, bacteria- or virus-infected cells, inflammatory cells, activated macrophages capable of causing a disease state, and any other type of pathogenic cells that uniquely express, preferentially express, or overexpress binding sites for the targeting agents described herein.

Illustratively, the population of pathogenic cells can be a cancer cell population that is tumorigenic, including benign tumors and malignant tumors, or it can be non-tumorigenic. The cancer cell population can arise spontaneously or by such processes as mutations present in the germline of the host animal or somatic mutations, or it can be chemically-, virally-, or radiation-induced. The invention can be utilized to diagnose, monitor, and/or treat such cancers, including carcinomas, sarcomas, lymphomas, Hodgekin's disease, melanomas, mesotheliomas, Burkitt's lymphoma, nasopharyngeal carcinomas, leukemias, and myelomas. The cancer cell population can include, but is not limited to, oral, thyroid, endocrine, skin, gastric, esophageal, laryngeal, pancreatic, colon, bladder, bone, ovarian, cervical, uterine, breast, testicular, prostate, rectal, kidney, liver, and lung cancers.

Illustratively, the population of pathogenic cells can also be activated monocytes or macrophages associated with disease states such as fibromyalgia, rheumatoid arthritis, osteoarthritis, ulcerative colitis, Crohn's disease, psoriasis, osteomyelitis, multiple sclerosis, atherosclerosis, pulmonary fibrosis, sarcoidosis, systemic sclerosis, organ transplant rejection (GVHD), lupus erythematosus, Sjögren's syndrome, glomerulonephritis, inflammations of the skin, such as psoriasis, and the like, chronic inflammations, and inflammations due to injury, such as head or spinal cord injury, embolisms, and the like.

The conjugates described herein can be formed from, for example, a wide variety of vitamins or receptor-binding vitamin analogs/derivatives, linkers, and imaging and radiotherapy agents. The conjugates described herein are capable of selectively targeting a population of pathogenic cells in the host animal due to preferential expression of a receptor for the targeting agent, such as a vitamin, accessible for binding, on the pathogenic cells. Illustrative vitamin moieties that can be used as the targeting agent (B) include carnitine, inositol, lipoic acid, pyridoxal, ascorbic acid, niacin, pantothenic acid, folic acid, riboflavin, thiamine, biotin, vitamin B12, and the lipid soluble vitamins A, D, E and K. These vitamins, and their receptor-binding analogs and derivatives, constitute an illustrative targeting entity that can be coupled with the imaging or radiotherapy agent by a bivalent linker (L) to form a targeting agent (B) imaging or radiotherapy agent conjugate as described herein. The term vitamin is understood to include vitamin analogs and/or derivatives, unless otherwise indicated. Illustratively, pteroic acid which is a derivative of folate, biotin analogs such as biocytin, biotin sulfoxide, oxybiotin and other biotin receptor-binding compounds, and the like, are considered to be vitamins, vitamin analogs, and vitamin derivatives. It should be appreciated that vitamin analogs or derivatives as described herein refer to vitamins that incorporates an heteroatom through which the vitamin analog or derivative is covalently bound to the bivalent linker (L).

Illustrative vitamin moieties include folic acid, biotin, riboflavin, thiamine, vitamin B₁₂, and receptor-binding analogs and derivatives of these vitamin molecules, and other related vitamin receptor binding molecules.

In one embodiment, the targeting group B is a folate, an analog of folate, or a derivative of folate. It is to be understood as used herein, that the term folate is used both individually and collectively to refer to folic acid itself, and/or to such analogs and derivatives of folic acid that are capable of binding to folate receptors.

Illustrative embodiments of vitamin analogs and/or derivatives include folate and analogs and derivatives of folate such as folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refer to the art-recognized analogs having a carbon atom substituted for one or two nitrogen atoms in the naturally occurring folic acid structure, or analog or derivative thereof. For example, the deaza analogs include the 1-deaza, 3-deaza, 5-deaza, 8-deaza, and 10-deaza analogs of folate, folinic acid, pteropolyglutamic acid, and folate receptor-binding pteridines such as tetrahydropterins, dihydrofolates, and tetrahydrofolates. The dideaza analogs include, for example, 1,5-dideaza, 5,10-dideaza, 8,10-dideaza, and 5,8-dideaza analogs of folate, folinic acid, pteropolyglutamic acid, and folate receptor-binding pteridines such as tetrahydropterins, dihydrofolates, and tetrahydrofolates. Other folates useful as complex forming ligands for this invention are the folate receptor-binding analogs aminopterin, amethopterin (also known as methotrexate), N10-methylfolate, 2-deamino-hydroxyfolate, deaza analogs such as 1-deazamethopterin or 3-deazamethopterin, and 3′,5′-dichloro-4-amino-4-deoxy-N10-methylpteroylglutamic acid (dichloromethotrexate). The foregoing folic acid analogs and/or derivatives are conventionally termed “folates,” reflecting their ability to bind with folate-receptors, and such ligands when conjugated with exogenous molecules are effective to enhance transmembrane transport, such as via folate-mediated endocytosis as described herein.

Additional analogs of folic acid that bind to folic acid receptors are described in US Patent Application Publication Serial Nos. 2005/0227985 and 2004/0242582, the disclosures of which are incorporated herein by reference. Illustratively, radicals of such folate analogs have the general formula:

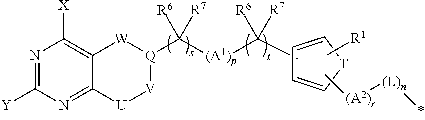

wherein
X and Y are each—independently selected from the group consisting of halo, R², OR², SR³, and NR⁴R⁵;

U, V, and W represent divalent moieties each independently selected from the group consisting of (R^{6a})C═, N═, (R^{6a})C(R^{7a}), and N(R^{4a});
Q is selected from the group consisting of C and CH;
T is selected from the group consisting of S, O, N, NH, and —C≡C—;
A¹ and A² are each independently selected from the group consisting of oxygen, sulfur, C(Z), C(Z)O, OC(Z), N(R^{4b}), C(Z)N(R^{4b}), N(R^{4b})C(Z), OC(Z)N(R^{4b}), N(R^{4b})C(Z)O, N(R^{4b})C(Z)N(R^{5b}), S(O), S(O)₂, N(R^{4a}) S(O)₂, C(R^{6b})(R^{7b}), N(C≡CH), N(CH₂C≡CH), C₁-C₁₂ alkylene, and C₁-C₁₂ alkyeneoxy, where Z is oxygen or sulfur;
R¹ is selected-from the group consisting of hydrogen, halo, C₁-C₁₂ alkyl, and C₁-C₁₂ alkoxy; R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy) carbonyl, and (C₁-C₁₂ alkylamino) carbonyl;
R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;
L is one or more, such as 1, 2, 3, 4, 5, 6, 7, 8, 9, or 10, amino acids; and
n, p, r, s and t are each independently either 0 or 1.

As used herein, it is to be understood that the term folate refers both individually to folic acid used in forming a conjugate, or alternatively to a folate analog or derivative thereof that is capable of binding to folate or folic acid receptors.

In another embodiment, the targeting group is a PSMA ligand or inhibitor, such as a derivative of pentanedioic acid of the formula:

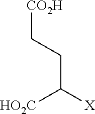

wherein X is RP(O)(OH)CH₂—(U.S. Pat. No. 5,968,915); RP(O)(OH)N(R¹)—(U.S. Pat. No. 5,863,536); RP(O)(OH)O—(U.S. Pat. No. 5,795,877); RN(OH)C(O)Y—or RC(O)NH(OH)Y, wherein Y is —CR₁R₂—, —NR³—or —O—(U.S. Pat. No. 5,962,521); RS(O)Y, RSO₂Y, or RS(O)(NH)Y, wherein Y is —CR₁R₂—, —NR³—or —O—(U.S. Pat. No. 5,902,817); and RS-alkyl, wherein R is for example hydrogen, alkyl, aryl, or arylalkyl, each of which may be optionally substituted (J. Med. Chem. 46:1989-1996 (2003)).

In each of the foregoing formulae, R, R₁, R₂, and R₃ are each independently selected from hydrogen, C₁-C₉ straight or branched chain alkyl, C₂-C₉ straight or branched chain alkenyl, C₃-C₈ cycloalkyl, C₅-C₇ cycloalkenyl, and aryl. In addition, in each case, each of R, R₁, R₂, and R₃ may be optionally substituted, such as with one or more groups selected from C₃-C₈ cycloalkyl, C₅-C₇ cycloalkenyl, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, amino, aryl. In one aspect, aryl is selected from 1-naphthyl, 2-naphthyl, 2-indolyl, 3-indolyl, 2-furyl, 3-furyl, 2-thienyl, 3-thienyl, 2-pyridyl, 3-pyridyl, 4-pyridyl, benzyl, and phenyl, and in each case aryl may be optionally substituted with one or more, illustratively with one to three, groups selected from halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino. In one variation of each of the above formulae, R is not hydrogen.

Illustrative PSMA ligands (U.S. Pat. No. 5,968,915) include
2-[methylhydroxyphosphinyl]methyl]pentanedioic acid;
2-[ethylhydroxyphosphinyl]methyl]pentanedioic acid;
2-[propylhydroxyphosphinyl]methyl]pentanedioic acid;
2-[butylhydroxyphosphinyl]methyl]pentanedioic acid;
2-[cyclohexylhydroxyphosphinyl]methyl]pentanedioic acid;
2-[phenylhydroxyphosphinyl]methyl]pentanedioic acid;
2-[2-(tetrahydrofuranyl) hydroxyphosphinyl]methyl]pentanedioic acid;
2-[(2-tetrahydropyranyl) hydroxyphosphinyl]methyl]pentanedioic acid;
2-[[((4-pyridyl)methyl) hydroxyphosphinyl]methyl]pentanedioic acid;
2-[[((2-pyridyl)methyl) hydroxyphosphinyl]methyl]pentanedioic acid;
2-[(phenylmethyl) hydroxyphosphinyl]methyl]pentanedioic acid;
2-[[((2-phenylethyl)methyl) hydroxyphosphinyl]methyl]pentanedioic acid;
2-[[((3-phenylpropyl)methyl) hydroxyphosphinyl]methyl]pentanedioic acid;
2-[[((3-phenylbutyl)methyl) hydroxyphosphinyl]methyl]pentanedioic acid;
2-[[((2-phenylbutyl)methyl) hydroxyphosphinyl]methyl]pentanedioic acid;
2-[(4-phenylbutyl) hydroxyphosphinyl]methyl]pentanedioic acid; and
2-[[(aminomethyl) hydroxyphosphinyl]methyl]pentanedioic acid.

Illustrative PSMA ligands (U.S. Pat. No. 5,863,536) include
N-[methylhydroxyphosphinyl] glutamic acid; N-[ethylhydroxyphosphinyl] glutamic acid;
N-[propylhydroxyphosphinyl] glutamic acid; N-[butylhydroxyphosphinyl] glutamic acid;
N-[phenylhydroxyphosphinyl] glutamic acid; N-[(phenylmethyl) hydroxyphosphinyl] glutamic acid; N-[((2-phenylethyl)methyl) hydroxyphosphinyl] glutamic acid; and
N-methyl-N-[phenylhydroxyphosphinyl] glutamic acid.

Illustrative PSMA ligands (U.S. Pat. No. 5,795,877) include
2-[methylhydroxyphosphinyl] oxy]pentanedioic acid;
2-[ethylhydroxyphosphinyl] oxy]pentanedioic acid;
2-[propylhydroxyphosphinyl] oxy]pentanedioic acid;
2-[[butylhydroxyphosphinyl] oxy]pentanedioic acid;
2-[[phenylhydroxyphosphinyl] oxy]pentanedioic acid;
2-[[((4-pyridyl)methyl) hydroxyphosphinyl] oxy]pentanedioic acid;
2-[[((2-pyridyl)methyl) hydroxyphosphinyl] oxy]pentanedioic acid;
2-[(phenylmethyl) hydroxyphosphinyl] oxy]pentanedioic acid; and
2 [[((2-phenylethyl)methyl) hydroxyphosphinyl] oxy]pentanedioic acid.

Illustrative PSMA ligands (U.S. Pat. No. 5,962,521) include
2-[(N-hydroxy) carbamoyl]methyl]pentanedioic acid;
2-[(N-hydroxy-N-methyl) carbamoyl]methyl]pentanedioic acid; 2-[(N-butyl-N-hydroxy) carbamoyl]methyl]pentanedioic acid; 2-[[(N-benzyl-N-hydroxy) carbamoyl]methyl]pentanedioic acid; 2-[(N-hydroxy-N-phenyl) carbamoyl]methyl]pentanedioic acid;
2-[[(N-hydroxy-N-2-phenylethyl) carbamoyl]methyl]pentanedioic acid; 2-[(N-ethyl-N-hydroxy) carbamoyl]methyl]pentanedioic acid; 2-[(N-hydroxy-N-propyl) carbamoyl]methyl]pentanedioic acid; 2-[(N-hydroxy-N-3-phenylpropyl) carbamoyl]methyl]pentanedioic acid;
2-[[(N-hydroxy-N-4-pyridyl) carbamoyl]methyl]pentanedioic acid;
2-[[(N-hydroxy) carboxamido]methyl]pentanedioic acid; 2-[[N-hydroxy(methyl) carboxamido]methyl]pentanedioic acid; 2-[[N-hydroxy (benzyl) carboxamido]methyl]pentanedioic acid;
2-[N-hydroxy (phenyl) carboxamido]methyl]pentanedioic acid;
2-[N-hydroxy (2-phenylethyl) carboxamido]methyl]pentanedioic acid;
2-[N-hydroxy (ethyl) carboxamido]methyl]pentanedioic acid; 2-[N-hydroxy (propyl) carboxamido]methyl]pentanedioic acid; 2-[[N-hydroxy (3-phenylpropyl) carboxamido]methyl]pentanedioic acid; and
2-[[N-hydroxy (4-pyridyl) carboxamido]methyl]pentanedioic acid.

Illustrative PSMA ligands (U.S. Pat. No. 5,902,817) include
2-[(sulfinyl)methyl]pentanedioic acid; 2-[(methylsulfinyl)methyl]pentanedioic acid;
2-[(ethylsulfinyl)methyl]pentanedioic acid; 2-[(propylsulfinyl)methyl]pentanedioic acid;
2-[(butylsulfinyl)methyl]pentanedioic acid; 2-[(phenylsulfinyl]methyl]pentanedioic acid; 2-[(2-phenylethyl) sulfinyl]methyl]pentanedioic acid;
2-[[(3-phenylpropyl) sulfinyl]methyl]pentanedioic acid;
2-[[(4-pyridyl) sulfinyl]methyl]pentanedioic acid; 2-[(benzylsulfinyl)methyl]pentanedioic acid; 2-[(sulfonyl)methyl]pentanedioic acid; 2-[(methylsulfonyl)methyl]pentanedioic acid;
2-[(ethylsulfonyl)methyl]pentanedioic acid; 2-[(propylsulfonyl)methyl]pentanedioic acid;
2-[(butylsulfonyl)methyl]pentanedioic acid; 2-[(phenylsulfonyl]methyl]pentanedioic acid; 2-[[(2-phenylethyl) sulfonyl]methyl]pentanedioic acid;
2-[(3-phenylpropyl) sulfonyl]methyl]pentanedioic acid; 2-[(4-pyridyl) sulfonyl]methyl]pentanedioic acid; 2-[(benzylsulfonyl)methyl]pentanedioic acid;
2-[(sulfoximinyl)methyl]pentanedioic acid; 2-[(methylsulfoximinyl)methyl]pentanedioic acid; 2-[(ethylsulfoximinyl)methyl]pentanedioic acid; 2-[(propylsulfoximinyl)methyl]pentanedioic acid;
2-[(butylsulfoximinyl)methyl]pentanedioic acid; 2-[(phenylsulfoximinyl]methyl]pentanedioic acid; 2-[(2-phenylethyl) sulfoximinyl]methyl]pentanedioic acid; 2-[(3-phenylpropyl) sulfoximinyl]methyl]pentanedioic acid; 2-[(4-pyridyl) sulfoximinyl]methyl]pentanedioic acid; and 2-[(benzylsulfoximinyl)methyl]pentanedioic acid.

Illustrative PSMA ligands include

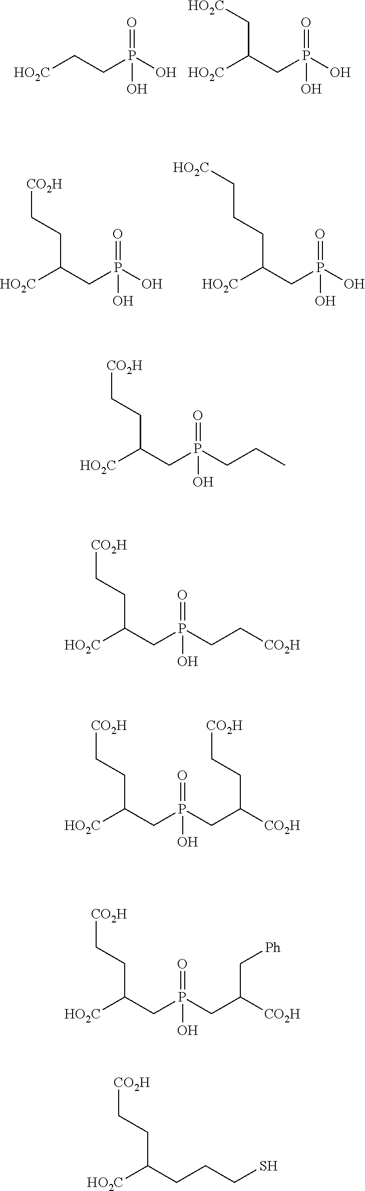

In another embodiment, the PSMA ligand is a urea of two amino acids. In one aspect, the amino acids include one or more additional carboxylic acids. In another embodiment, the amino acids include one or more additional phosphoric, phosphonic, phosphinic, sulfinic, sulfonic, or boronic acids. In another aspect, the amino acids include one or more thiol groups or derivatives thereof. In another aspect, the amino acids include one or more carboxylic acid bioisosteres, such as tetrazoles and the like.

In another embodiment, the PSMA ligand is a compound of the formula:

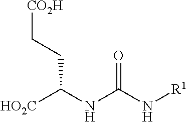

where R¹ is

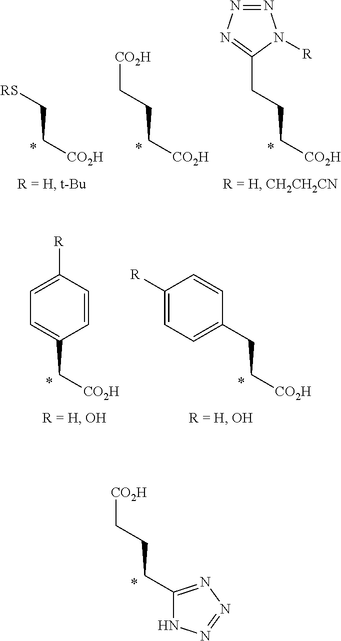

In another illustrative embodiment, the binding agent is a urea of an amino dicarboxylic acid, such as aspartic acid, glutamic acid, and the like, and another amino dicarboxylic acid, or an analog thereof, such as a binding agent of the formulae

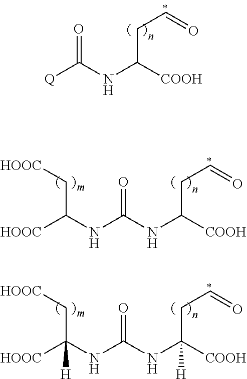

wherein Q is a an amino dicarboxylic acid, such as aspartic acid, glutamic acid, or an analog thereof, n and m are each independently selected from an integer between 1 and about 6, and (*) represents the point of attachment for the linker L.

Illustratively, the PSMA ligand is a compound of the formulae:

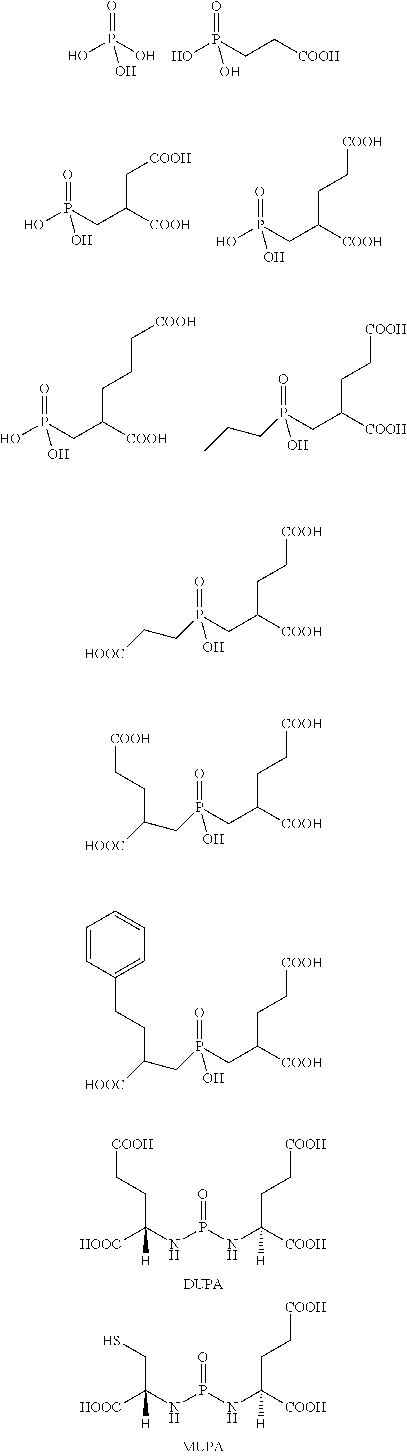

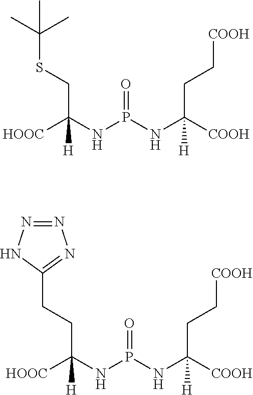

In another embodiment, the PSMA ligand is 2-[3-(1-Carboxy-2-mercapto-ethyl)-ureido]-pentanedioic acid (MUPA) or 2-[3-(1,3-Dicarboxy-propyl)-ureido]-pentanedioic acid (DUPA).

Other illustrative examples of PSMA ligands include peptide analogs such as quisqualic acid, aspartate glutamate (Asp-Glu), Glu-Glu, Gly-Glu, γ-Glu-Glu, beta-N-acetyl-L-aspartate-L-glutamate (β-NAAG), and the like.

In another embodiment, the PSMA ligand comprises a urea or thiourea of lysine and an amino acid, or one or more carboxylic acid derivatives thereof, including, but not limited to ureas or thioureas of lysine and aspartic acid, or glutamic acid, or homoglutamic acid.

In another embodiment, the PSMA ligand comprises a urea or thiourea of L-lysine and L-glutamate.

In another embodiment, the PSMA ligand comprises a compound selected from the following

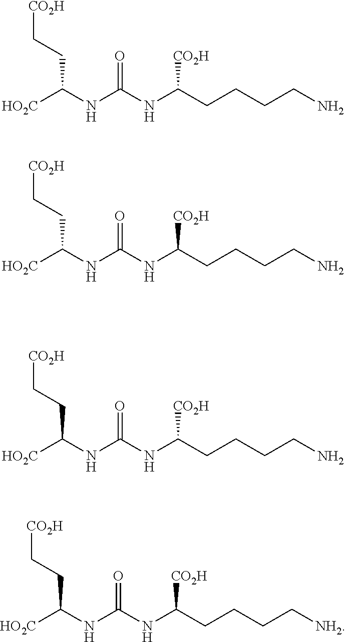 [SMILES: NCCCC[C@H](NC(=O)N[C@@H](CCC(=O)O)C(=O)O)C(=O)O ; NCCCC[C@@H](NC(=O)N[C@@H](CCC(=O)O)C(=O)O)C(=O)O ; NCCCC[C@H](NC(=O)N[C@H](CCC(=O)O)C(=O)O)C(=O)O ; NCCCC[C@@H](NC(=O)N[C@H](CCC(=O)O)C(=O)O)C(=O)O]

In another embodiment, the PSMA ligand comprises the following

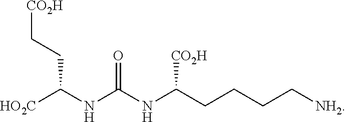 [SMILES: NCCCC[C@H](NC(=O)N[C@@H](CCC(=O)O)C(=O)O)C(=O)O]

The compounds, linkers, intermediates, and conjugates described herein may be prepared using conventional processes, including the described in International Patent Publication Nos. WO 2009/002993, WO 2004/069159, WO 2007/022494, and WO 2006/012527, and U.S. patent application Ser. No. 13/837,539 (filed Mar. 15, 2013). The disclosures of each of the foregoing are herein incorporated by reference in their entirety.

Each publication cited herein is incorporated herein by reference.

In another embodiment, a method is described for diagnosing and/or monitoring a disease or disease state where the method comprises the steps of administering to a patient being evaluated for the disease state an effective amount of a conjugate of the general formula B-L-P. The method includes allowing sufficient time for the conjugate to bind to the target tissue, and diagnosing and/or monitoring the disease or disease state extra-corporeally, such as by using positron emission tomography.

The radionuclide may include a positron-emitting isotope having a suitable half-life and toxicity profile. In various embodiments, the radioisotope has a half-life of more than 30 minutes, more than 70 minutes, more than 80 minutes, more than 90 minutes, more than 100 minutes, less than 8 hours, less than 6 hours, less than 4 hours, or less than 3 hours. In other embodiments, the radioisotope has a half-life of about 30 minutes to about 4 hours, about 70 minutes to about 4 hours, about 80 minutes to about 4 hours, about 90 minutes to about 4 hours, about 100 minutes to about 4 hours, about 30 minutes to about 6 hours, about 70 minutes to about 6 hours, about 80 minutes to about 6 hours, about 90 minutes to about 6 hours, about 100 minutes to about 6 hours, about 30 minutes to about 8 hours, about 70 minutes to about 8 hours, about 80 minutes to about 8 hours, about 90 minutes to about 8 hours, or about 100 minutes to about 8 hours.

The radionuclide may include one or more positron-emitting isotopes, such as but not limited to isotopes selected from ⁸⁹Zr, ⁴⁵Ti, ⁵¹Mn, ⁶⁴Cu, ⁶¹Cu, ⁶³Zn, ⁸²Rb, ⁶⁸Ga, ⁶⁶Ga, ¹¹C, ¹³N, ¹⁵O, ¹²⁴I, ³⁴Cl, and ¹⁸F. In another embodiment, the radionuclide is a halide, such as a positron-emitting halide. In another embodiment, the radionuclide is a metal ion, such as a positron-emitting metal ion. In another embodiment, the radionuclide is a gallium ion, such as a positron-emitting gallium ion. In another embodiment, the radionuclide is selected from ⁸⁹Zr, ⁶⁴Cu, ⁶⁸Ga, ⁶⁶Ga, 124I, and ¹⁸F. In another illustrative embodiment, the radioisotope is selected from ⁸⁹Zr, ⁶⁴Cu, ⁶⁸Ga, 124I, and ¹⁸F. In another embodiment, the radioisotope is ⁶⁸Ga, or ⁸⁹Zr, or ¹⁸F. In another embodiment in each of the foregoing and following embodiments described herein, the radioisotope is ⁶⁸Ga. In another embodiment in each of the foregoing and following embodiments described herein, the radioisotope is ¹⁸F. In another embodiment in each of the foregoing and following embodiments described herein, the radioisotope is ⁸⁹Zr. In another embodiment in each of the foregoing and following embodiments described herein, the radioisotope is ⁶⁴Cu. It is also to be understood that the fluorine isotopes described herein may be selected from various isotopic combinations of ¹⁸F and ¹⁹F. It is understood that factors that may be included during selection of a suitable isotope include sufficient half-life of the positron-emitting isotope to permit preparation of a diagnostic composition in a pharmaceutically acceptable carrier prior to administration to the patient, and sufficient remaining half-life to yield sufficient activity to permit extra-corporeal measurement by a PET scan. Further, a suitable isotope should have a sufficiently short half-life to limit patient exposure to unnecessary radiation. In an illustrative embodiment, ¹⁸F, having a half-life of 110 minutes, provides adequate time for preparation of the diagnostic composition, as well as an acceptable deterioration rate. Further, on decay ¹⁸F is converted to 180.

Illustrative positron-decaying isotopes having suitable half-lives include ³⁴Cl, half-life about 32 minutes; ⁴⁵Ti, half-life about 3 hours; ⁵¹Mn, half-life about 45 minutes; ⁶¹Cu, half-life about 3.4 hours; ⁶³Zn, half-life about 38 minutes; ⁸²Rb, half-life about 2 minutes; ⁶⁸Ga, half-life about 68 minutes, ⁶⁶Ga, half-life about 9.5 hours, ¹¹C, half-life about 20 minutes, ¹⁵O, half-life about 2 minutes, ¹³N, half-life about 10 minutes, or ¹⁸F, half-life about 110 minutes. In another embodiment, the radionuclide is a radiotherapy agent. Illustrative radionuclides for radiotherapy include isotopes of lutetium such as ¹⁷⁷Lu, isotopes of yttrium, such as ⁹⁰Y, isotopes of copper, such as ⁶⁷Cu and ⁶⁴Cu, and the like.

The radionuclide may be covalently attached to the conjugate, such as to an aryl or heteroaryl aromatic group, including benzamidyl, benzylic, phenyl, pyridinyl, pyrimidinyl, pyridazinyl, naphthyl, benzothiazolyl, benzimizolyl, benzoxazolyl, and like groups. In one illustrative embodiment, the radioisotope is ¹⁸F and the radionuclide includes an aryl group to which the radioisotope is covalently attached.

The radionuclide may be non-covalently attached to the conjugate, such as within a chelate.

The methods may also be used in combination with any other methods of cancer diagnosis already developed and known in the art, including methods using other already developed diagnostic agents and utilizing x-ray computed tomography (CT), magnetic resonance imaging (MRI), functional magnetic resonance imaging (fMRI), ultrasound, and single photon emission computed tomography (SPECT).

It is understood that in certain applications of the methods described herein, each of the processes and synthetic methods described herein either substantially complete fluorination, or alternatively only partial fluorination may be desired. Accordingly, the processes and synthetic methods described herein may be performed in various alternative embodiments. It is therefore understood that in those aspects where only partial fluorination is desired, the processes and syntheses described herein may be performed with less than stoichiometric amounts of fluorinating agent. Similarly, it is understood that in certain applications of the methods described herein, each of the processes and synthetic methods described herein either substantially complete radiofluorination, or alternatively only partial radiofluorination may be desired. Accordingly, the processes and synthetic methods described herein may be performed in various alternative embodiments. It is therefore understood that in those aspects where only partial radiofluorination is desired, the processes and syntheses described herein may be performed with less than stoichiometric amounts of radiofluorination agent, where the balance is optionally ¹⁹F.

The following examples further illustrate specific embodiments of the invention; however, the following illustrative examples should not be interpreted in any way to limit the invention.

### EXAMPLES

General. Water was distilled and then deionized (18 MQ/cm²) by passing through a Milli-Q water filtration system (Millipore Corp., Milford, MA). All chemicals and solvents, unless specified, were purchased from Sigma (St. Louis, MO) and were used without further purification. Amino acids were purchased from Chem-Impex Int (Chicago, IL). 2,2′-(7-(2-((2,5-dioxopyrrolidin-1-yl)oxy)-2-oxoethyl)-1,4,7-triazonane-1,4-diyl)diacetic acid (NOTA-NHS) was purchased from CheMatech (France). N10-TFA-Pteroic Acid was provided by Endocyte, Inc. High-performance liquid chromatography (HPLC) analysis and purification of the DUPA-NOTA precursor were performed on an Agilent G6130B instrument. The radioactive HPLC was performed with a γ-counter using a Xselect CSH C18 (250×10 mm) column and MeCN and 0.1% Formic Acid as mobile phases.

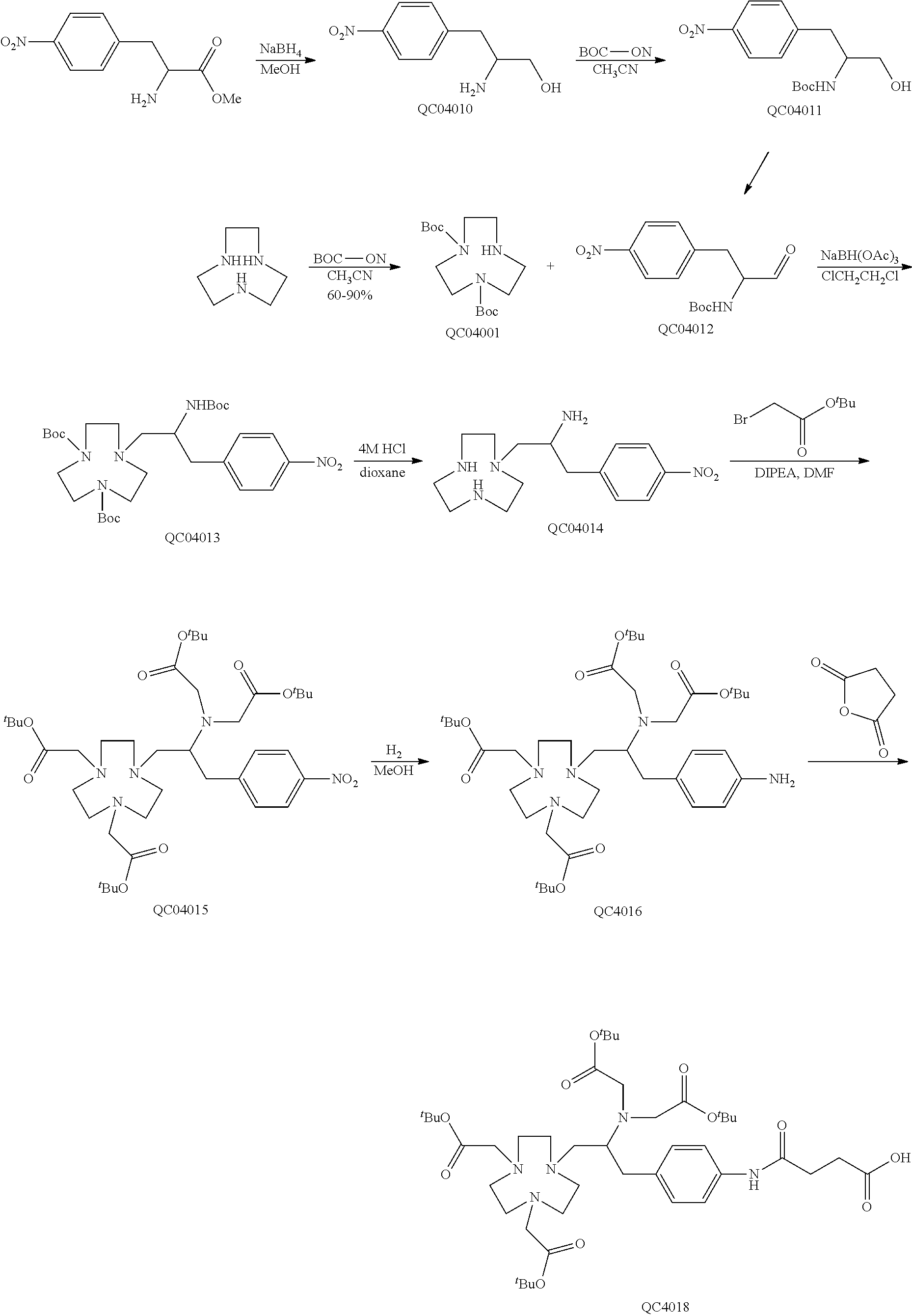

EXAMPLE. C-NETA. tert-Butyl [2-Hydroxy-1-(4-nitrobenzyl)ethyl]carbamate (QC04011) was prepared from the commercially available methyl 2-amino-3-(4-nitrophenyl) propanoate through NaBH₄ reduction and Boc-protection. Successive Dess-Martin oxidation and reductive amination with QC04001 afforded tris-Boc protected compound QC04013, which was transformed to QC04014 after Boc-deprotection in 4 M HCl in dioxane. Treatment of QC04014 with tert-butyl bromoacetate, followed by hydrogenolysis of the NO₂ group provided QC04016. Further reaction of QC04016 with succinic anhydride provided the bifunctional C-NETA (QC04018) as the corresponding tert-butyl ester.

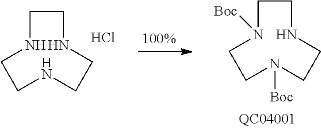 [SMILES: C1CNCCNCCN1 ; Cl ; CC(C)(C)OC(=O)N1CCNCCN(C(=O)OC(C)(C)C)CC1]

EXAMPLE. Di-tert-butyl [1,4,7]Triazanonane-1,4-dicarboxylate (QC04001). QC04001 was prepared according to a modification of a synthetic procedure reported previously. [19-21] To a solution of 1,4,7-triazonane trihydrogenchloride (TACN.3HCl, 1.85 g, 7.7 mmol, M.W.: 238.6) in CHCl₃ (25 mL) was added DIPEA (4.0 mL, 3.0 g, 23.1 mmol, M.W.: 129.24, d: 0.742) and BOC—ON(3.77 g, 15.3 mmol, M.W.: 246.26) in portions. The resulting mixture was stirred for 5 days and the solvent evaporated under vacuum. The residue was partitioned between 10% NaOH solution (10 mL) and diethyl ether (30 mL). The ether layer was separated and washed with 10% NaOH solution (10 mL) and water (10 mL) several times. The ether layer was dried (MgSO₄), filtered, and concentrated under vacuum to provide QC04001 (2.53 g, quantitative), which was used without further purification. ¹H NMR (400 MHZ, CDCl₃) δ=3.47-3.50 (m, 2H), 3.42-3.45 (m, 2H), 3.38 (br, s, 1H), 3.28-3.34 (m, 2H), 3.16-3.28 (m, 2H), 2.86-2.99 (m, 4H), 1.48 (s, 18H); ¹³C NMR (101 MHZ, CDCl₃) δ=156.08, 155.85 (C═O), 79.80, 79.70 (“Bu), 53.20, 52.62, 52.52, 51.78, 50.50, 49.91, 49.63, 48.39, 48.23, 47.83, 47.46 (TACN ring from 53.20-47.46), 28.60 (^{t-}Bu).

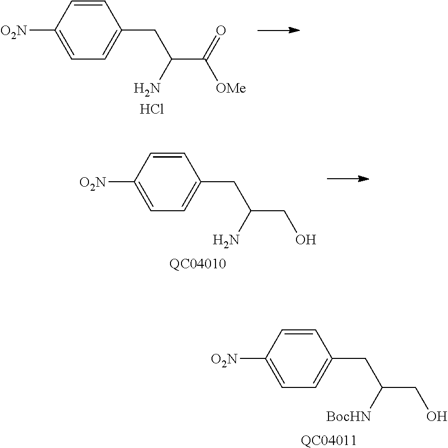 [SMILES: COC(=O)C(N)Cc1ccc([N+](=O)[O-])cc1 ; Cl ; NC(CO)Cc1ccc([N+](=O)[O-])cc1 ; "QC04010" ; CC(C)(C)OC(=O)NC(CO)Cc1ccc([N+](=O)[O-])cc1 ; "QC04011"]

EXAMPLE. tert-Butyl [2-Hydroxy-1-(4-nitrobenzyl)ethyl]carbamate (QC04011) [19]. With minor revision to the reported procedure, where the HCl salt of methyl 2-amino-3-(4-nitrophenyl) propanoate was used directly without neutralization with Et3N, to a solution of the methyl 2-amino-3-(4-nitrophenyl) propanoate hydrochloride salt (6.22 g, 23.9 mmol) in MeOH(70 mL) at 23° C. was added NaBH₄ (2.86 g, 71.4 mmol) in multiple portions. The reaction was monitored by TLC and LC-MS. The mixture was heated to reflux (with water bath at ˜ 70° C.), and NaBH₄ was added portion-wise as needed until most of the starting material disappeared, requiring about 6 grams of NaBH₄ in total. After evaporation of the solvent, the residue was treated with H₂O (70 mL) and extracted with DCM/IPA (3/1). The combined organic layer was dried, filtered, and concentrated under vacuum to provide white solid QC04010 (4.4 g, 94), which was used without further purification.

EXAMPLE. QC04010 (4.4 g, 22.7 mmol) was dissolved in CH₃CN(30 mL) at ambient temperature, to which was added BOC—ON(11.2 g, 27.2 mmol, 1.2 eq.) portionwise. To the above mixture was added DIPEA (5.24 mL, 3.76 g, 29.2 mmol, M.W.: 129.24, d: 0.742), the resulting mixture was stirred for 4 h and evaporated. The residue was partitioned between ether (50 mL) and 10% NaOH solution (20 mL). The ether layer was separated and washed with 10% NaOH solution (10 mL) and water (10 mL) sequentially. The ether layer was dried, filtered, and concentrated under vacuum. The residue was washed with ether (20 mL) to provide QC04011 (5.31 g, 75%), which was used without further purification. To prepare an analytical sample, the residue is purified via column chromatography on SiO₂ eluting with Hexane/Ethyl Acetate (3/1 to 1/1 with 1% of MeOH) to afford pure QC04011 as a white solid. ¹H NMR (400 MHZ, CDCl₃)>=8.15 (d, J=8.8 MHz, 2H), 7.40 (d, J=8.8 MHz, 2H), 4.84 (d, J=6.8 MHz, 1H), 3.90 (s, 1H), 3.68 (dd, J=3.1 MHz, 1H), 3.57 (dd, J=3.1 MHz, 1H), 2.98 (d, J=6.0 MHz, 2H), 1.39 (s, 9H); ¹³C NMR (101 MHz, CDCl₃) δ=156.0, 146.4, 146.2, 130.1, 123.5, 79.8, 63.3, 53.1, 37.3, 28.0.

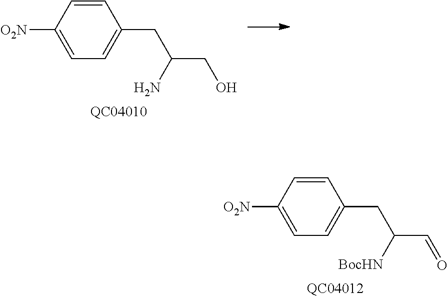 [SMILES: NC(CO)Cc1ccc([N+](=O)[O-])cc1 ; "QC04010" ; CC(C)(C)OC(=O)NC(C=O)Cc1ccc([N+](=O)[O-])cc1 ; "QC04012"]

EXAMPLE. tert-Butyl (1-(4-nitrophenyl)-3-oxopropan-2-yl) carbamate. QC04011 (1.27 g, 4.3 mmol) was dissolved in CH₂Cl2 (40 mL), and cooled to 0° C., to which Dess-Martin periodinane (1.70 g, 5.16 mmol, 1.2 equiv) was added in one portion. After stirring for 15 min at 0° C., the reaction was warmed to 23° C. and stirred for 45 min. The reaction was quenched by addition of a basic aq Na₂S₂O₃ solution (50/50, v/v of aq Na₂S₂O₃ and aq Na₂HCO₃), and the resulting mixture was vigorously stirred for 15 min. After extraction with CH₂Cl₂ (3×), the organic phases were washed successively with water and brine, dried overNa₂SO₄, filtered and concentrated in vacuo to provide QC04012, which was used without further purification.

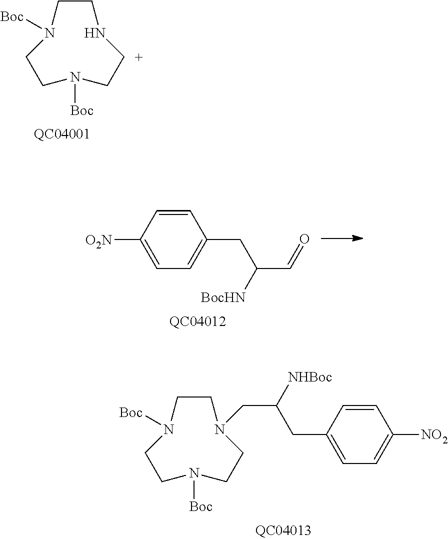

EXAMPLE. Reductive amination of QC04012 and QC04001 to prepare QC04013:₄. 1,4-Di-tert-butyl 7-(2- {[(tert-Butoxy) carbonyl]amino}3-(4-nitrophenyl) propyl)-1,4,7-triazonane-1,4-dicarboxylate (QC04013): Compound QC04012 (4.3 mmol in theory) was added to a solution of QC04001 (1.40 g, 4.3 mmol) in DCE (100 mL) at 0° C. . . . The resulting solution was stirred for 10 min and sodium triacetoxyborohydride (1.28 g, 6.02 mmol, 1.4 eq.) was added portionwise to the solution over 30 min. The mixture was stirred at ambient temperature overnight. The reaction mixture was concentrated, treated with a saturated aqueous solution of NaHCO₃ (50 mL), and extracted with ethyl acetate (3×50 mL). The combined organic layers were dried with Na₂SO₄, filtered, and concentrated in vacuo. The residue was purified via flash chromatography (SiO₂, Hex/EA=3/1) to provide QC04013 (2.31 g, 88.5% for 2 steps, based on 2.61 g in theory) as a pale yellow semi-solid. ¹H NMR (400 MHZ, CDCl₃) δ=8.11 (2H, d, J=7.6 Hz), 7.35 (2H, d, J=7.6 Hz), 5.28 (1H, s, br), 3.54-3.88 (2H, m), 3.39-3.54 (2H, m), 3.32-3.40 (1H, m), 3.15-3.32 (2H, m), 2.79-3.15 (4H, m), 2.37-2.73 (6H, m), 1.43 (9H, s), 1.42 (9H, s), 1.38 (9H, s); ¹³C NMR (101 MHz, CDCl₃) δ=156.15, 155.99, 155.70, 155.56, 147.00, 146.95, 146.81, 146.76, 130.36, 123.73, 123.65, 123.60, 80.07, 79.99, 79.92, 79.81, 79.57, 79.46, 60.79, 60.47, 55.52, 54.33, 54.06, 53.64, 53.15, 53.28, 51.54, 50.80, 50.71, 50.42, 49.87, 49.07, 48.12, 39.67, 39.45, 28.74, 28.61. MS m/z: MS-API: Calcd. for C₃₀H₅₀N₅O₈ ([M+H]⁺): 608.4, Found: 608.3;

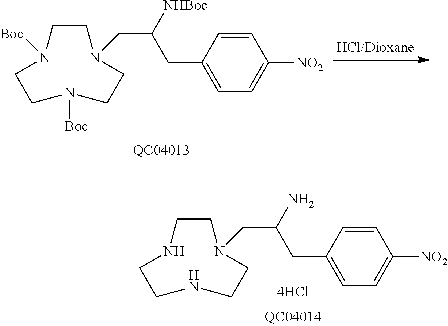 [SMILES: CC(C)(C)OC(=O)NC(Cc1ccc([N+](=O)[O-])cc1)CN1CCN(C(=O)OC(C)(C)C)CCN(C(=O)OC(C)(C)C)CC1 ; NC(Cc1ccc([N+](=O)[O-])cc1)CN1CCNCCNCC1]

EXAMPLE. 1-(4-Nitrophenyl)-3-(1,4,7-triazonan-1-yl) propan-2-amine. QC04013 (2.31 g, 3.8 mmol) was dispersed in 30 mL of 4 M HCl/Dioxane, the resulting mixture was stirred at room temperature for 20 hours. The reaction mixture was rapidly added to cold Et2O to precipitate a white solid. The solid was collected and dried in air to afford the pure product QC04014 (1.71 g, in quantitative yield) as a pale-white solid. MS m/z: MS-API: Calcd. for C₁₅H₂₆N₅O₂ ([M+H]⁺): 308.2, Found: 308.2;

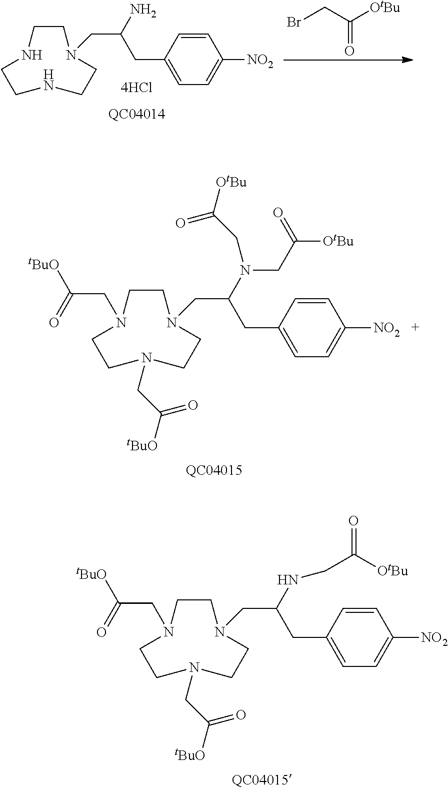

EXAMPLE. Introduction of the tri-tert-butyl ethylacetate^{1b}. To a solution of QC04014 (78 mg, 0.19 mmol) and DIPEA (0.272 mL, 202 mg, 1.56 mmol, 8.2 eq. M.W.: 129.24, d: 0.742) in DMF (2 mL) was added Nal (233.8 mg, 1.56 mmol, 8.2 eq. M.W.: 149.89) and tert-Butyl bromoacetate (0.126 mL, 168 mg, 0.86 mmol, 4.5 eq. M.W.: 195.05, d: 1.321) slowly at room temperature. The resulting mixture was warmed to 60-70° C. and stirred for 20 hs. After completion, monitored by TLC and LC-MS, the reaction was quenched by water and extracted with Et20. The combined organic solvent was washed successively with water and brine, and dried over Na₂SO₄. After filtration, the solvent was evaporated under vacuum, and resulting deep-colored oil residue was purified by flash chromatography on SiO₂ (DCM/MeOH═100/1-100/4) to provide QC04015 (14 mg, 10%) as a yellow oil and QC04015′ (61 mg, 49.4%). MS m/z: MS-API: Calcd. for C₃₉H₆₆N₅O₁₀ ([M+H]⁺): 764.5, Found: 764.4;

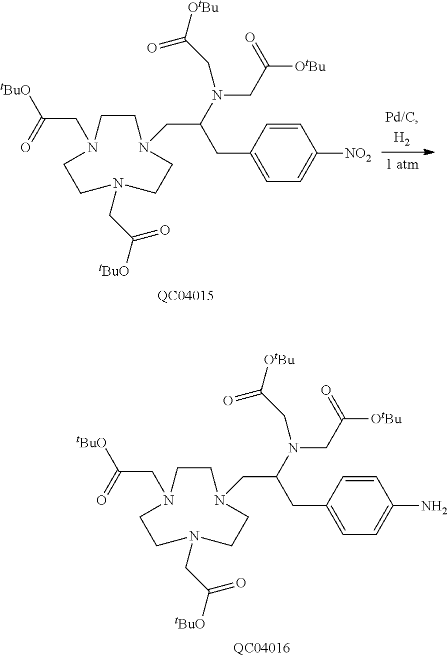 [SMILES: CC(C)(C)OC(=O)CN1CCN(CC(=O)OC(C)(C)C)CCN(CC(Cc2ccc([N+](=O)[O-])cc2)N(CC(=O)OC(C)(C)C)CC(=O)OC(C)(C)C)CC1 ; CC(C)(C)OC(=O)CN1CCN(CC(=O)OC(C)(C)C)CCN(CC(Cc2ccc(N)cc2)N(CC(=O)OC(C)(C)C)CC(=O)OC(C)(C)C)CC1]

EXAMPLE. To a solution of QC04015 (20 mg, 0.039 mmol) in MeOH(2 mL) was added 10% Pd/C catalyst (5 mg). The resulting mixture was subjected to hydrogenolysis by agitation with H₂ (g) at 1 atm (˜ 15 psi) at ambient temperature for 14 h. The reaction mixture was diluted with excess DCM and filtered through celite, and the filtrate was concentrated in vacuo to provide QC04016 (13 mg, 67.5%). MS m/z: MS-API: Calcd. for C₃₉H₆₈N₅O₈ ([M+H]⁺): 734.5, Found: 734.4;

### FOLATE TARGETED EXAMPLES

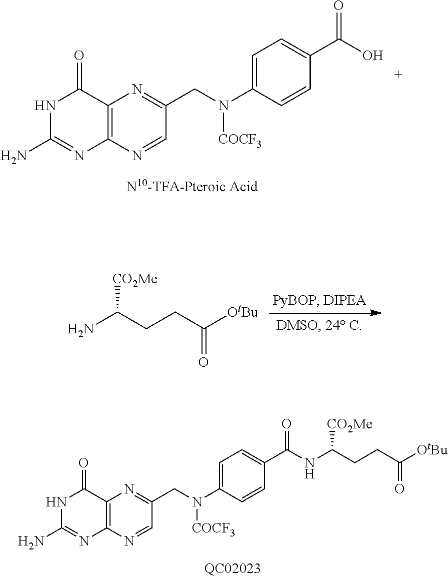

EXAMPLE. (S)-5-tert-butyl 1-methyl 2-(4-(N-((2-amino-4-oxo-3,4-dihydropteridin-6-yl)methyl)-2,2,2-trifluoroacetamido)benzamido) pentanedioate (QC02023). HCl·H₂N-Glu (OtBu)-OMe (350 mg, 1.38 mmol) was added to a solution of N10-TFA-Pteroic Acid (560 mg, 1.37 mmol) and DIPEA (1.2 mL, 6.85 mmol) in DMSO (6.0 mL) at 23° C. under N₂. After stirring for 15 min at 23° C., PyBOP (720 mg, 1.0 mmol) was added, and the reaction mixture was stirred for 24 h at 23° C. Volatile material was removed under reduced vacuum to afford the crude product as a semi-solid, which was further purified via solid extraction with Hex/EA (1/1) 3 times to provide QC02023 as a pale-yellow solid in quantitative yield, which was used without further purification. λₘₐₓ=280 nm; LC-MS(Agilent G6130B Quadrupole LC/MS): Mobile phase: Buffer (pH 7)—CH₃CN; Column: Analytic C18 column; Method: 0-100 CH3CN-15 min, t_{R}=5.62 min. MS m/z: MS-API: Calcd. for C₂₆H₂₉F₃N₇O₇ ([M+H]⁺): 608.2, Found: 608.1;

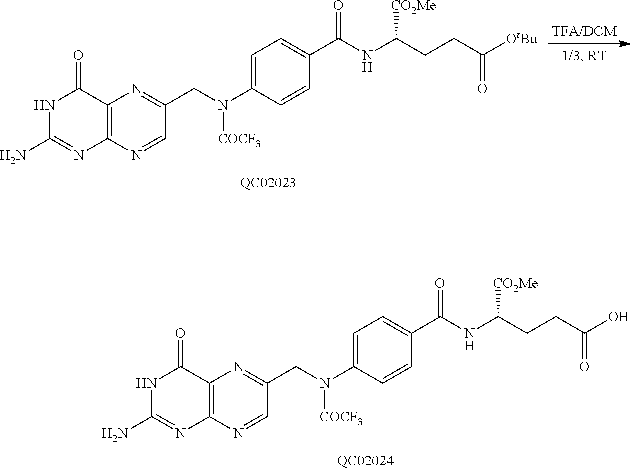

EXAMPLE. (S)-4-(4-(N-((2-amino-4-oxo-3,4-dihydropteridin-6-yl)methyl)-2,2,2-trifluoroacetamido)benzamido)-5-methoxy-5-oxopentanoic acid (QC02024). 224 mg of QC02023 was treated with TFA/DCM (15 mL, ⅓) at 23° C. The reaction was stirred at 23° C. and monitored by TLC. After 1.5 hours, starting material was not observed by TLC. The volatile material was removed under reduced pressure resulting in a semi-solid residue, which was treated with cold Et2O, to provide a pale white solid precipitate, which was collected by filtration and dried in air to provide(S)-4-(4-(N-((2-amino-4-oxo-3,4-dihydropteridin-6-yl)methyl)-2,2,2-trifluoroacetamido)benzamido)-5-methoxy-5-oxopentanoic acid QC02024 (169 mg, 83% for 2 steps). λₘₐₓ=280 nm; LC-MS(Agilent G6130B Quadrupole LC/MS): Mobile phase: Buffer (pH 7)—CH3CN; Column: Analytic C18 column; Method: 0-100 CH3CN-15 min, t_{R}=3.40 min. MS m/z: MS-API: Calcd. for C₂₂H₂₁F₃N₇O₇ ([M+H]⁺): 552.1, Found: 552.1; ¹H NMR (400 MHZ, DMSO) δ=12.16 (s, br, 1H), 8.88 (d, J=7.2 Hz, 1H), 8.65 (s, 1H), 7.92 (d, J=8.0 Hz, 2H), 7.64 (d, J=8.0 Hz, 2H), 7.16 (s, br, 1H), 5.14 (s, 2H), 4.38-4.55 (m, 1H), 3.64 (s, 3H), 2.28-2.40 (m, 2H), 2.00-2.12 (m, 1H), 1.87-2.00 (m, 1H); ¹³C NMR (101 MHZ, DMSO) δ=173.91, 172.36, 165.93, 161.03, 156.11, 155.76 (d, J=35.8 Hz), 154.19, 149.40, 144.45, 141.80, 134.30, 128.89, 128.62, 128.29, 117.91 (d, J=48.5 Hz), 53.90, 52.23, 52.06, 30.26, 25.81; 19F NMR (377 MHz, CDCl₃) δ=−62.87.

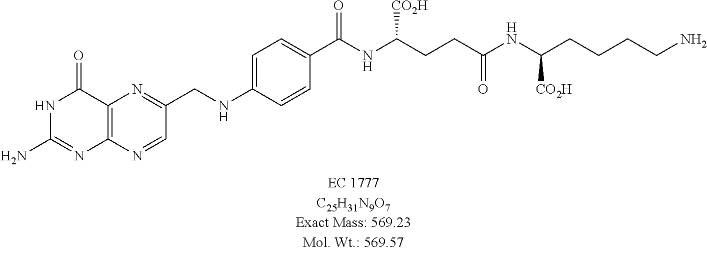 [SMILES: NCCCC[C@H](NC(=O)CC[C@H](NC(=O)c1ccc(NCc2cnc3nc(N)[nH]c(=O)c3n2)cc1)C(=O)O)C(=O)O]

EXAMPLE. Pte-γGlu-Lys-OH(EC1777). EC1777 was prepared using solid phase peptide synthesis as follows.

| | | | Molecular | Quantity |
|---|---|---|---|---|
| Compound | mmol | Equivalent | Weight | (grams) |
| Fmoc-Lys-Resin | 0.5 | 1 | | 1.00 |
| (Loading ~0.5 mmol/g) | | | | |
| Fmoc-Glu-O^{t}Bu | 1.0 | 2 | 425.5 | 0.426 |
| N¹⁰-TFA-Pteroic Acid | 0.65 | 1.3 | 408 | 0.265 |
| PyBOP | 1.3 | 2 | 520.31 | 0.52 |
| DIPEA | 1.5 | 3 | 129.24 | 0.168 |
| | | | (d = 0.742) | |

In a peptide synthesis vessel, Fmoc-Lys-resin (1.0 g, 0.5 mmol) was placed and washed with DMF (3×10 ml). Initial Fmoc deprotection was performed using 20% piperidine in DMF (3×10 ml) solution for 10 mins per cycle. Subsequent washes of DMF (3×10 ml) and i-PrOH(3×10 ml), a Kaiser test was done to determine reaction completion. Following another DMF wash (3×10 ml); an amino acid solution (2.0 eq.) in DMF, PyBOP (2.0 eq.) and DIPEA (3.0 eq.) were added to the vessel and the solution bubbled with Argon for 1 hour. The coupling solution was filtered, the resin was washed with DMF (3×10 ml) and i-PrOH(3×10 ml) and a Kaiser test was done to assess reaction completion. The above process was performed successively for the additional coupling. Resin cleavage was performed with a cocktail consisting of 95% CF3CO2H, 2.5% H2O and 2.5% triisopropylsilane. The cleavage cocktail (10 ml) was poured onto the resin and bubbled with Argon for 30 mins, followed by filtration into a clean flask. Further cleavage was performed twice successively with fresh cleavage cocktail for 10 mins of bubbling. The combined filtrate was poured onto cold diethyl ether, the precipitate formed was collected by centrifugation at 4000 rpm for 5 mins (3×). The precipitate was obtained following decanting and drying of the solid under vacuum. Deprotection of the trifluoro-acetyl group was achieved by dissolving the crude precipitate in H2O (15 ml), which was basified with Na₂CO3 to pH 9 with Argon bubbling. Upon completion of the reaction, confirmed by LCMS, the solution was acidified to pH 3 using 2 M HCl and the desired linker was purified by preparative HPLC(mobile phase A=10 mM Ammonium acetate, pH=5; Organic phase B=Acetonitrile; Method; 10% B to 100% B in 30 mins) to yield EC 1777 (112 mg, 39%); ¹H NMR (500 MHz DMSO-d₆) Pivotal signals: δ 8.60 (s, 1H), 7.58 (d, 2H), 6.60 (d, 2H), 4.45 (s, 2H). [M+H]+=Calculated 570.23, found 570.582

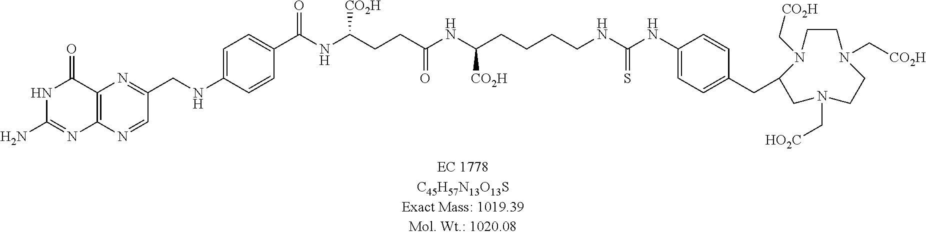 [SMILES: Nc1nc2ncc(CNc3ccc(C(=O)N[C@@H](CCC(=O)N[C@@H](CCCCNC(=S)Nc4ccc(CC5CN(CC(=O)O)CCN(CC(=O)O)CCN5CC(=O)O)cc4)C(=O)O)C(=O)O)cc3)nc2c(=O)[nH]1]

EXAMPLE. Pte-γGlu-Lys-NOTA. In a dry flask, EC 1777 (30.5 mg, 0.054 mmol, 1.0 eq.), 1,1,3,3-tetramethylguanidine (13.45 μl, 0.107 mmol, 2.0 eq.) and DMSO (2.5 ml) under Argon were sonicated for 1 hour. DIPEA (0.19 ml, 1.07 mmol, 20 eq.) was added to the solution, followed by sonication for an addition hour. To the transparent solution was added p-SCN-Bn-NOTA.3HCl (33 mg, 0.059 mmol, 1.1 eq.) and the reaction was monitored until completion by LCMS and purified using preparative HPLC(mobile phase A=10 mM Ammonium acetate, pH=5; Organic phase B=Acetonitrile; Method; 10% B to 100% B in 30 mins) to yield EC 1778 (16 mg, 29%). ¹H NMR (500 MHz DMSO-d₆) Pivotal signals: δ 8.60 (s, 1H), 7.58 (d, 2H), 7.29 (d, 2H), 7.07 (d, 2H), 6.61 (d, 2H), 4.45 (s, 2H), 4.20 (t, 1H). [M+H]+=Calculated 1020.39, found 1020.63.

EXAMPLE. Pte-γGlu-Lys-NOTA-A1-¹⁸F is prepared by reaction of Pte-γGlu-Lys-NOTA with Al¹⁸F₃·3H₂O (1 step method) or with AlCl₃·3H₂O followed by reaction with Na¹⁸F (2 step method) using published processes.

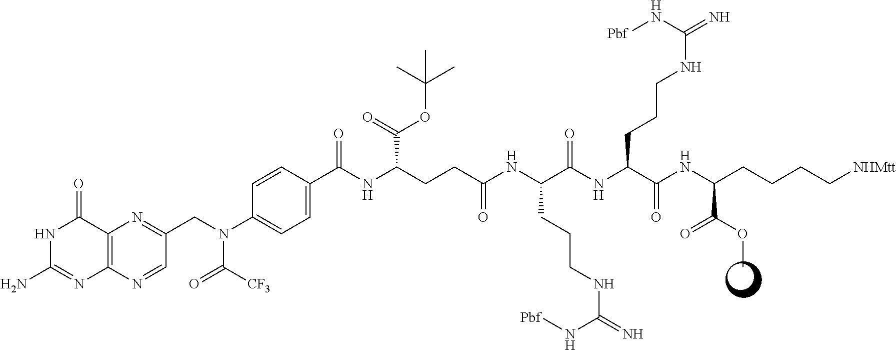

EXAMPLE. N10-TFA-Pte-γGlu-OtBu-Arg (Pbf)-Arg (Pbf)-Lys(Mtt)-resin 3. The general procedure described for the synthesis of resin bound folate-peptide resin 1 was followed for the coupling of 2× Fmoc-L-Arg (Pbf)-OH, Fmoc-Glu-OtBu, and N10-TFA-Pte-OH to Fmoc-L-Lys(Mtt)-Wang resin.

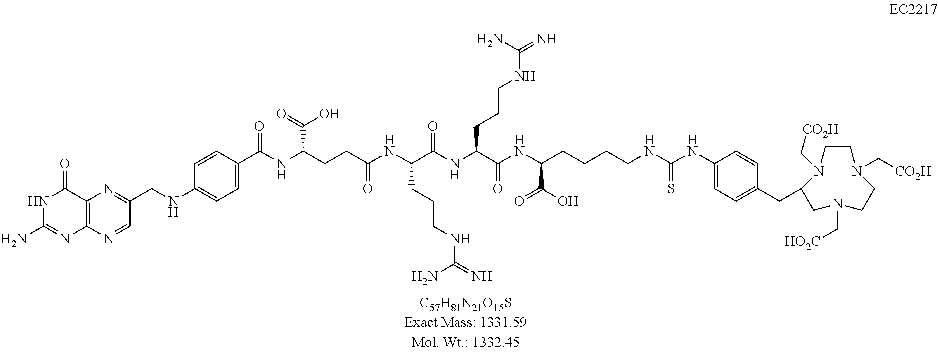 [SMILES: N=C(N)NCCC[C@H](NC(=O)CC[C@H](NC(=O)c1ccc(NCc2cnc3nc(N)[nH]c(=O)c3n2)cc1)C(=O)O)C(=O)N[C@@H](CCCNC(=N)N)C(=O)N[C@@H](CCCCNC(=S)Nc1ccc(CC2CN(CC(=O)O)CCN(CC(=O)O)CCN2CC(=O)O)cc1)C(=O)O]

EXAMPLE. Pte-γGlu-Arg-Arg-Lys-Bn-NOTA 4 (EC2217). In a peptide synthesis vessel, N10-TFA-Pte-γGlu-OtBu-Arg (Pbf)-Arg (Pbf)-Lys(Mtt)-resin (0.28 g, 0.07 mmol) was placed and washed with DCM (3×10 ml). Selective Mtt deprotection was performed by adding a 2% CF₃CO₂H/DCM solution to the vessel and bubbling with Argon for 10 min. After filtering, the resin was washed with dichloromethane followed by a fresh solution of 2% CF₃CO₂H/DCM. This process was repeated until there was no more yellow solution being yielded and a Kaiser test was done. Following a DMF wash (3×10 ml); p-SCN-Bn-NOTA·3HCl (50 mg, 0.09 mmol, 1.2 eq.) in DMF, and DIPEA (80 μl, 0.45 mmol, 6.0 eq.) were added to the vessel and the solution bubbled with Argon for 2 hour. The coupling solution was filtered, the resin was washed with DMF (3×10 ml) and i-PrOH(3×10 ml) and a Kaiser test was done to assess reaction completion. Resin cleavage/global tert-butyl ester deprotection was performed with a cocktail consisting of 95% CF₃CO₂H, 2.5% H₂O and 2.5% triisopropylsilane. The cleavage cocktail (10 ml) was poured onto the resin and bubbled with Argon for 60 mins, followed by filtration into a clean flask. Further cleavage was performed twice successively with fresh cleavage cocktail for 20 mins of bubbling. The combined filtrate was poured onto cold diethyl ether, the precipitate formed was collected by centrifugation at 4000 rpm for 5 mins (3×). The precipitate was obtained following decanting and drying of the solid under vacuum. Deprotection of the trifluoro-acetyl group was achieved by dissolving the crude precipitate in H2O (15 ml), which was basified with Na₂CO₃ to pH 9 with Argon bubbling. Upon completion of the reaction, confirmed by LCMS, the solution was acidified to pH 5 using 2 M HCl and the desired linker was purified by preparative HPLC(mobile phase A=10 mM Ammonium acetate, pH=5; Organic phase B=Acetonitrile; Method; 10% B to 100% B in 30 mins) to yield EC2217 (35 mg, 35%). ¹H NMR (500 MHz DMSO-d₆) Pivotal signals: δ 8.61 (s, 1H), 7.54 (d, J=8.4 Hz, 2H), 7.17-7.03 (m, 2H), 6.99 (d, J=8.0 Hz, 2H), 6.66 (d, J=8.5 Hz, 2H), 4.52-4.45 (m, 1H), 4.17 (dt, J=8.9, 4.6 Hz, 2H), 4.12 (s, 1H), 4.07-3.97 (m, 1H). [M+H]+=Calculated 1332.59, found 1332.87

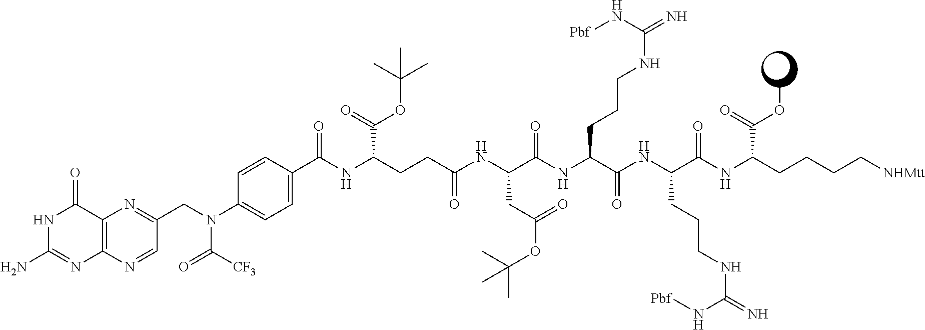

EXAMPLE. N10-TFA-Pte-γGlu-OtBu-Asp (OtBu)-Arg (Pbf)-Arg (Pbf)-Lys(Mtt)-resin 5. The general procedure described for the synthesis of resin bound folate-peptide resin 1 was followed for the coupling of 2× Fmoc-L-Arg (Pbf)-OH, Fmoc-L-Asp (OtBu)-OH, Fmoc-Glu-OtBu, and N10-TFA-Pte-OH to Fmoc-L-Lys(Mtt)-Wang resin.

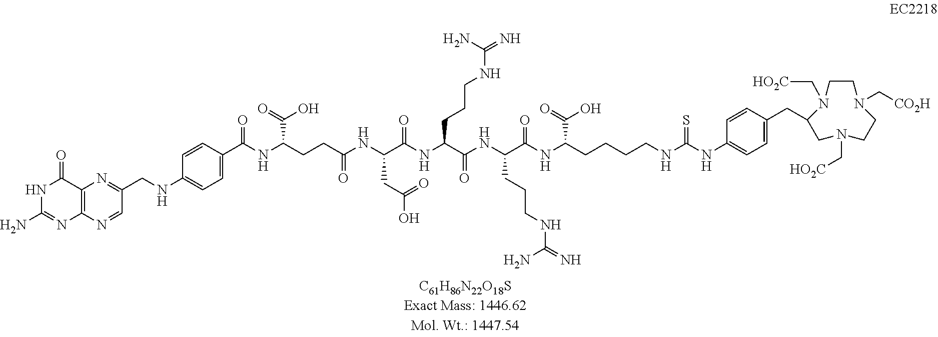 [SMILES: N=C(N)NCCC[C@H](NC(=O)[C@H](CCCNC(=N)N)NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CCCCNC(=S)Nc1ccc(CC2CN(CC(=O)O)CCN(CC(=O)O)CCN2CC(=O)O)cc1)C(=O)O]

EXAMPLE. Pte-γGlu-Asp-Arg-Arg-Lys-Bn-NOTA 6 (EC2218). Pte-γGlu-Asp-Arg-Arg-Lys-Bn-NOTA, EC2218 was prepared in 18% yield according to the process described for folate-peptide-NOTA, 4.¹H NMR (500 MHz DMSO-d₆) Pivotal signals: δ 8.58 (s, 1H), 7.52 (d, J=9.0 Hz, 2H), 7.14-7.08 (m, 4H), 6.61 (d, J=9.0 Hz, 2H), 4.16-4.09 (m, 2H), 4.06 (dd, J=10.0, 4.3 Hz, 1H), 3.90 (dd, J=7.8, 4.7 Hz, 1H). [M+H]+=Calculated 1449.64, found 1449.76

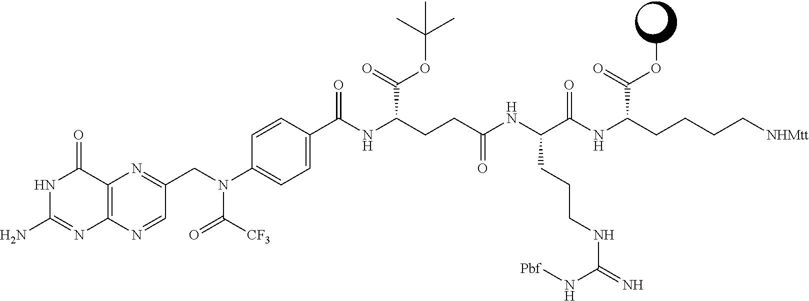

EXAMPLE. N10-TFA-Pte-γGlu-OtBu-Arg (Pbf)-Lys(Mtt)-resin 7. The general procedure described for the synthesis of resin bound folate-peptide resin 1 was followed for the coupling of Fmoc-L-Arg (Pbf)-OH, Fmoc-Glu-OtBu, and N10-TFA-Pte-OH to Fmoc-L-Lys(Mtt)-Wang resin.

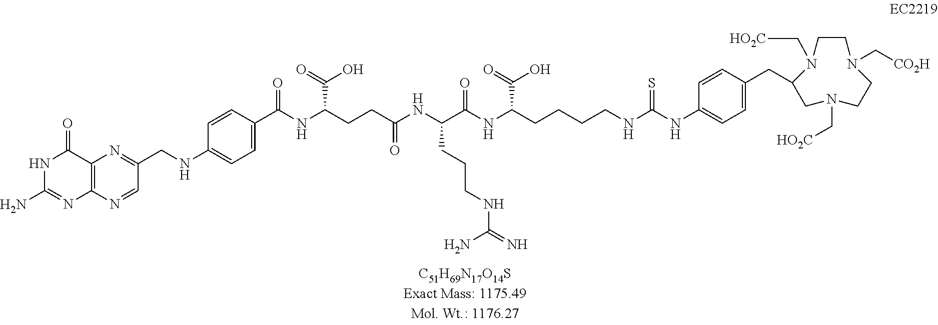 [SMILES: N=C(N)NCCC[C@H](NC(=O)CC[C@H](NC(=O)c1ccc(NCc2cnc3nc(N)[nH]c(=O)c3n2)cc1)C(=O)O)C(=O)N[C@@H](CCCCNC(=S)Nc1ccc(CC2CN(CC(=O)O)CCN(CC(=O)O)CCN2CC(=O)O)cc1)C(=O)O]

EXAMPLE. Pte-γGlu-Arg-Lys-Bn-NOTA 8 (EC2219). Pte-γGlu-Arg-Lys-Bn-NOTA, EC2219 was prepared in 20% yield according to the process described for folate-peptide-NOTA, 4.¹H NMR (500 MHz DMSO-d₆) Pivotal signals: δ 8.68 (s, 1H), 7.60 (d, J=8.4 Hz, 3H), 7.27-6.97 (m, 4H), 6.77-6.69 (m, 2H), 4.28-f 4.19 (m, 2H), 4.08 (dd, J=9.0, 5.4 Hz, 1H), 4.01 (dd, J=8.5, 5.4 Hz, 1H). [M+H]+=Calculated 1178.51, found 1178.7

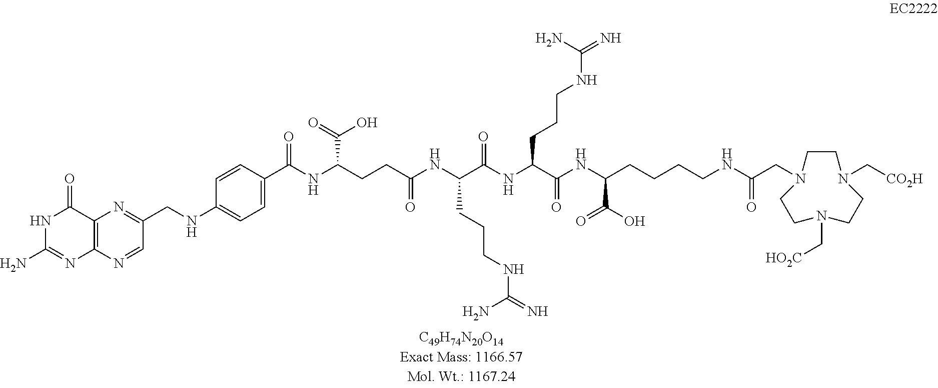 [SMILES: N=C(N)NCCC[C@H](NC(=O)CC[C@H](NC(=O)c1ccc(NCc2cnc3nc(N)[nH]c(=O)c3n2)cc1)C(=O)O)C(=O)N[C@@H](CCCNC(=N)N)C(=O)N[C@@H](CCCCNC(=O)CN1CCN(CC(=O)O)CCN(CC(=O)O)CC1)C(=O)O]

EXAMPLE. Pte-γGlu-Arg-Arg-Lys-NOTA 9 (EC2222). In a peptide synthesis vessel, N10-TFA-Pte-γGlu-OtBu-Arg (Pbf)-Arg (Pbf)-Lys(Mtt)-resin (0.5 g, 0.12 mmol) was placed and washed with DCM (3×10 ml). Selective Mtt deprotection was performed by adding a 2% CF₃CO₂H/DCM solution to the vessel and bubbling with Argon for 10 min. After filtering, the resin was washed with dichloromethane followed by a fresh solution of 2% CF₃CO₂H/DCM. This process was repeated until there was no more yellow solution being yielded and a Kaiser test was done. Following a DMF wash (3×10 ml); NOTA-Bis(tBu) ester (0.10 g, 0.24 mmol, 2.0 eq.) in DMF, PyBOP (0.14 g, 0.26 mmol, 2.2 eq) and DIPEA (64 μl, 0.36 mmol, 3.0 eq.) were added to the vessel and the solution bubbled with Argon for 2 hour. The coupling solution was filtered, the resin was washed with DMF (3×10 ml) and i-PrOH(3×10 ml) and a Kaiser test was done to assess reaction completion. Resin cleavage/global tert-butyl ester deprotection was performed with a cocktail consisting of 95% CF₃CO₂H, 2.5% H₂O and 2.5% triisopropylsilane. The cleavage cocktail (10 ml) was poured onto the resin and bubbled with Argon for 1 hr, followed by filtration into a clean flask. Further cleavage was performed twice successively with fresh cleavage cocktail for 10 mins of bubbling. The combined filtrate was poured onto cold diethyl ether, the precipitate formed was collected by centrifugation at 4000 rpm for 5 mins (3×). The precipitate was obtained following decanting and drying of the solid under vacuum. Deprotection of the trifluoro-acetyl group was achieved by dissolving the crude precipitate in H₂O (15 ml), which was basified with Na₂CO₃ to pH 9 with Argon bubbling. Upon completion of the reaction, confirmed by LCMS, the solution was acidified to pH 5 using 2 M HCl and the desired linker was purified by preparative HPLC(mobile phase A=10 mM Ammonium acetate, pH=5; Organic phase B=Acetonitrile; Method; 10% B to 100% B in 30 mins) to yield EC2222 (28 mg, 20%). ¹H NMR (500 MHz DMSO-d₆) Pivotal signals: δ 8.60 (s, 1H), 7.51 (d, J=8.1 Hz, 2H), 6.64 (d, J=8.4 Hz, 2H), 4.21-4.09 (m, 2H), 4.09-4.03 (m, 1H), 3.98-3.88 (m, 1H), 3.50 (s, 1H). [M+H]+=Calculated 1167.57, found 1167.8

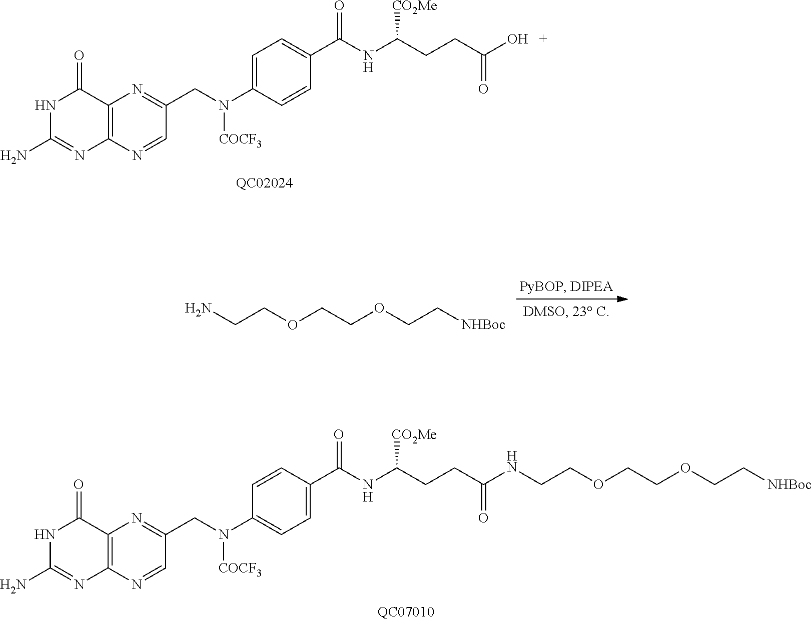

EXAMPLE. (S)-Methyl 18-(4-(N-((2-amino-4-oxo-3,4-dihydropteridin-6-yl)methyl)-2,2,2-trifluoroacetamido)benzamido)-2,2-dimethyl-4,15-dioxo-3,8,11-trioxa-5,14-diazanonadecan-19-oate (QC07010). QC02024 (100 mg, 0.181 mmol) is added to a solution of Mono-Boc-PEG-NH₂ (45 mg, 0.181 mmol) and DIPEA (0.158 mL, 0.905 mmol) in DMSO (2 mL) at 23° C. under N₂. After being stirred for 15 min at 23° C., PyBOP (94.2 mg, 0.181 mmol) was added, and the reaction mixture was stirred for 24 h at 23° C. Volatile material was removed under reduced vacuum, the crude material was further purified by SPE purification: extract successively with ACN(2×), EA (1×) and Et20 (1×) to afford pure product QC07010 (127 mg, 90%). λmax=280 nm; LC-MS(Agilent G6130B Quadrupole LC/MS): Mobile phase: Buffer (pH 7)—CH3CN; Column: Analytic C18 column; Method: 0-100 CH3CN-15 min, t_{R}=5.06 min. MS m/z: MS-API: Calcd. for C33H43F3N9O10 ([M+H]⁺): 782.3, Found: 782.2; 1H NMR (400 MHz, DMSO) δ=11.59 (s, br, 1H), 8.92 (d, J=7.2 Hz, 1H), 8.64 (s, 1H), 7.85-8.02 (m, 3H), 7.64 (d, J=8.0 Hz, 2H), 6.75 (t, J=5.2 Hz, 1H), 5.13 (s, 2H), 4.33-4.48 (m, 1H), 3.64 (s, 3H), 3.46 (s, 4H), 3.30-3.41 (s, 4H), 3.14-3.23 (m, 2H), 3.01-3.08 (m, 2H), 2.19-2.30 (m, 2H), 2.02-2.12 (m, 1H), 1.89-2.00 (m, 1H), 1.35 (s, 9H); ¹³C NMR (101 MHz, DMSO) δ=172.43, 171.46, 165.73, 160.87, 156.80, 155.70 (d, J=35.5 Hz), 155.67, 154.17, 149.49, 144.20, 141.73, 134.30, 128.82, 128.55, 128.23, 116.20 (d, J=290.0 Hz), 77.65, 69.58, 69.50, 69.193, 69.192, 53.88, 52.52, 51.96, 38.89, 38.62, 31.65, 28.23, 26.32; 19F NMR (377 MHz, CDCl₃) δ =−62.87.

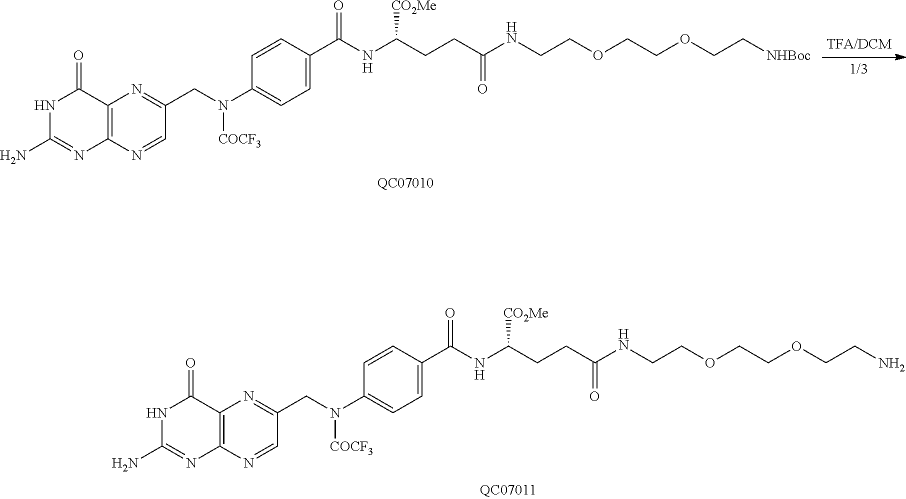

EXAMPLE. (S)-methyl 2-(4-(N-((2-amino-4-oxo-3,4-dihydropteridin-6-yl)methyl)-2,2,2-trifluoroacetamido)benzamido)-5-((2-(2-(2-aminoethoxy) ethoxy)ethyl) amino)-5-oxopentanoate (QC07011). QC07010 (274 mg, 0.35 mmol) was treated with TFA/DCM (4 mL, ⅓) at 23° C. The reaction was stirred at 23° C. and monitored by LC-MS. After 1.5 h, TLC showed that all starting material disappeared. The mixture was diluted with CH3CN and evaporated to dry via rota-vap. Residue TFA (b.p. 72.4° C.) was removed through azeotropic distillation with ACN to afford the product QC07011 in quantitative yield, which was used without further purification. λₘₐₓ=280 nm; LC-MS(Agilent G6130B Quadrupole LC/MS): Mobile phase: Buffer (pH 7)-ACN; Column: Analytic C18 column; Method: 0-100 ACN 15 min, t_{R}=3.84 min. MS m/z: MS-API: Calcd. for C₂₈H₃₅F₃N₉O₈ ([M+H]⁺): 682.2, Found: 682.2.

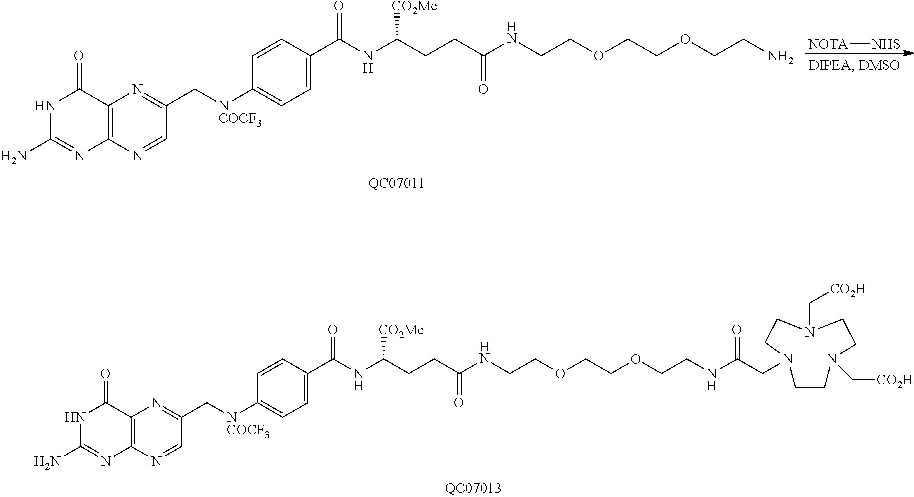

EXAMPLE. (S)-2,2′-(7-(4-(4-(N-((2-amino-4-oxo-3,4-dihydropteridin-6-yl)methyl)-2,2,2-trifluoroacetamido)benzamido)-3,7,18-trioxo-2,11,14-trioxa-8,17-diazanonadecan-19-yl)-1,4,7-triazonane-1,4-diyl)diacetic acid (QC07013). QC07011 (15.7 mg, 0.023 mmol) in DMSO (0.5 ml) was added NOTA-NHS(18.2 mg, 0.028 mmol) followed by DIPEA (15 μL, 0.084 mmol). The reaction was stirred at 23° C., monitored by LC-MS, and most of the starting material was converted to QC07013 in 5 hours. The product was purified by RP-C₁₈ HPLC to afford the pure product QC07013 (13.0 mg, 58.5%). λₘₐₓ=280 nm; LC-MS (Agilent G6130B Quadrupole LC/MS): Mobile phase: Buffer (pH 7)—CH3CN; Method: 0-100 CH3CN-15 min, t_{R}=3.74 min. MS m/z: MS-API: Calcd. for C₄₀H₅₄F₃N₁₂O₁₃ ([M+H]⁺): 967.4, Found: 967.2; HPLC(Agilent Preparative C18 Column): Mobile phase: Buffer (pH 7)—CH3CN; Method: 0-100 CH3CN-30 min, t_{R}=10.75 min

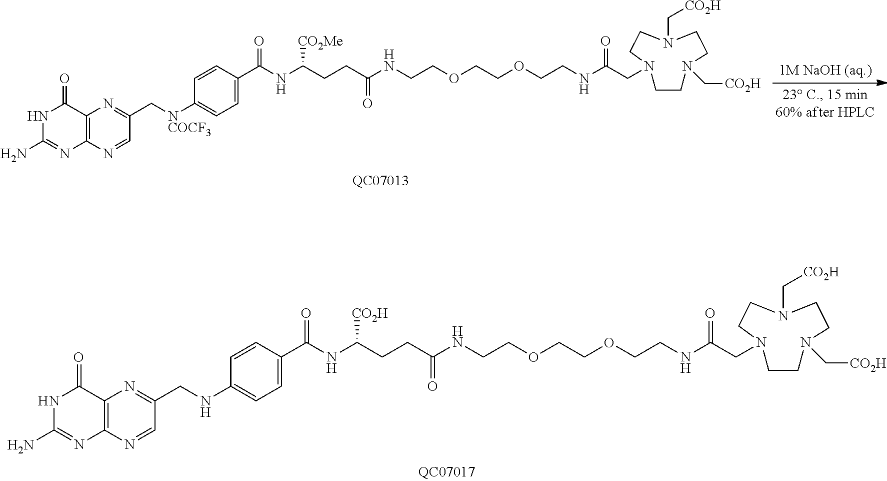

EXAMPLE. (S)-2,2′-(7-(1-(4-(((2-amino-4-oxo-3,4-dihydropteridin-6-yl)methyl)amino) phenyl)-3-carboxy-1,6,17-trioxo-10,13-dioxa-2,7,16-triazaoctadecan-18-yl)-1,4,7-triazonane-1,4-diyl)diacetic acid (FA-PEG1-NOTA, QC07017). QC07013 (20.8 mg, 0.022 mmol) was stirred in 1.2 mL of 1 M NaOH(aq.) at 23° C. and the reaction was monitored by LC-MS. After 15 min, all starting material was transformed to product, the crude material was purified by RP-C18 HPLC to afford QC07017 (11.3 mg, 60%). Δₘₐₓ=280 nm; HPLC (Agilent Preparative C18 Column): Mobile phase: Buffer (pH 7)—CH3CN; Method: 0-30 CH3CN-30 min, t_{R}=11.49 min. LC-MS(Agilent G6130B Quadrupole LC/MS): Mobile phase: Buffer (pH 7)—CH3CN; Method: 0-100 CH3CN 15 min, t_{R}=2.72 min. MS m/z: MS-API: Calcd. for C37H53N12O12 ([M+H]⁺): 857.4, Found: 857.2.¹H NMR (400 MHZ, DMSO) δ=8.62 (s, 1H), 8.28 (t, J=5.6 Hz, 1H), 7.99 (t, J=5.6 Hz, 1H), 7.85 (d, J=7.2 Hz, 1H), 7.76-7.80 (s, br, 2H), 7.58 (d, J=8.8 Hz, 2H), 7.00 (t, J=6.0 Hz, 1H), 6.62 (d, J=8.8 Hz, 2H), 4.47 (d, J=5.2 Hz, 2H), 4.13-4.18 (m, 1H), 3.43 (s, 4H), 3.31-3.41 (m, 4H), 3.29-3.32 (m, 2H), 3.10-3.24 (m, 4H), 3.03-3.10 (s, br, 2H), 2.90-3.03 (s, br, 2H), 2.10-2.14 (m, 2H), 1.97-2.05 (m, 1H), 1.84-1.91 (m, 1H); ¹³C NMR (101 MHz, DMSO) δ=174.33, 172.21, 171.17, 170.35, 165.70, 161.85, 156.19, 154.95, 150.56, 148.45, 148.32, 128.62, 127.87, 121.84, 111.38, 69.44, 69.30, 69.08, 68.70, 60.95, 57.48, 53.11, 50.85, 49.41, 48.91, 45.88, 38.60, 38.18, 32.04, 27.52.

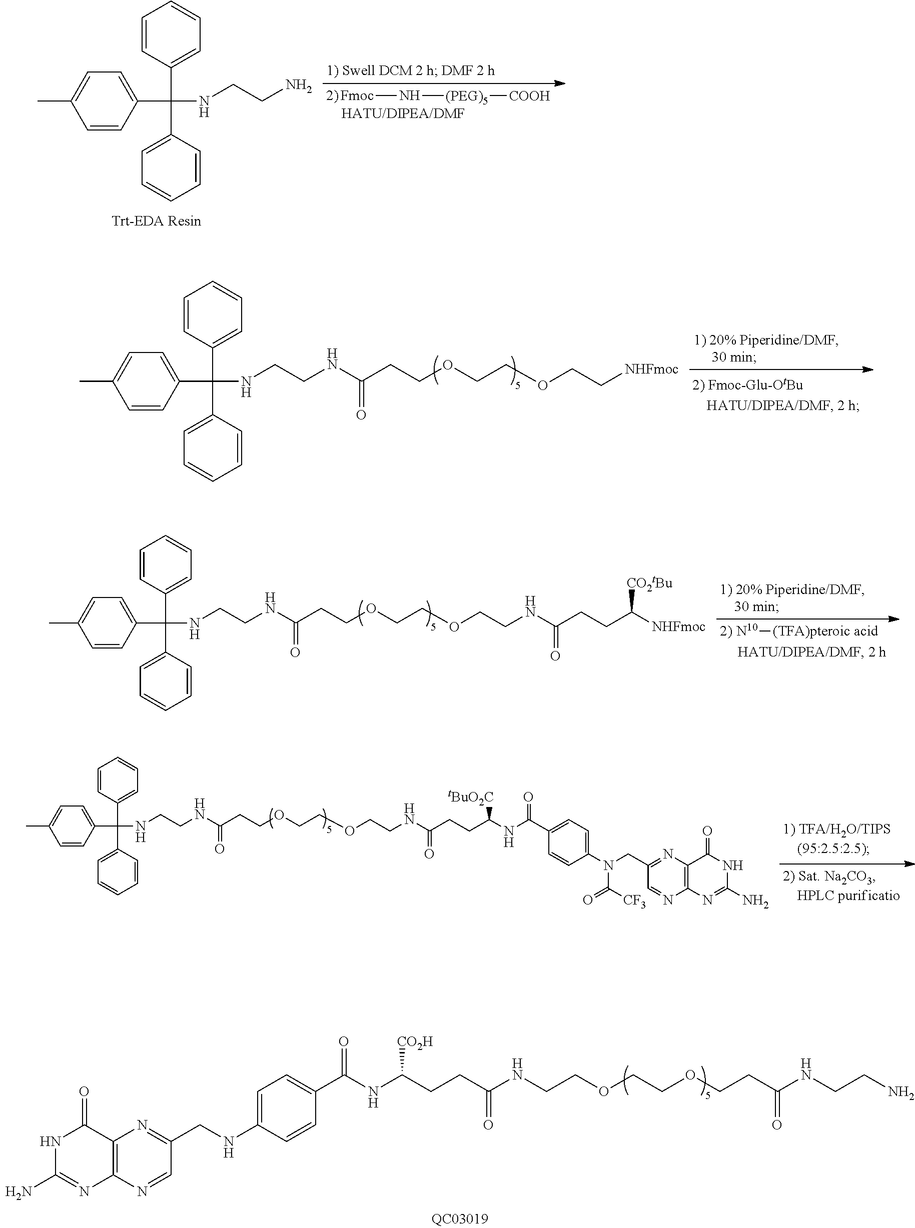

EXAMPLE. Solid Phase Synthesis (SPS) of FA-PEG₆-EDA-NH₂ Precursor (QC03019). 1,2-Diaminoethane trityl resin (1.2 mmol/g, 100 mg, 0.12 mmol) was swollen with dichloromethane (DCM, 3 mL) followed by dimethyl formamide (DMF, 3 mL). After swelling the resin in DMF, a solution of fluorenylmethoxycarbonyl (Fmoc)-PEG₆-OH(1.5 equiv), HATU (1.5 equiv), and DIPEA (2.0 equiv) in DMF was added. Argon was bubbled for 2 h, and resin was washed with DMF (3×3 mL) and i-PrOH(3× 3 mL). The above sequence was repeated for two more coupling steps for conjugation of Fmoc-Glu-(OtBu)-OH and N¹⁰-TFA-Ptc-OH. The final product was cleaved from the resin using a trifluoroacetic acid (TFA): H₂O: triisopropylsilane cocktail (95:2.5:2.5) and concentrated under vacuum. The concentrated product was precipitated in diethyl ether and dried under vacuum, which was then incubated in Sat. Na₂CO₃ and monitored by LC-MS. 1 hour later, the mixture was neutralized to pH=7 with 2 M HCl (aq.) which was purified by preparative with preparative RP-C₁₈ HPLC [solvent gradient: 0% B to 50% B in 30 min; A=10 mM NH₄OAc, pH=7; B═CH3CN]. Acetonitrile was removed under vacuum, and the residue was freeze-dried to yield QC03019 as a yellow solid (59 mg, 60%). Analytical RP-C₁₈ HPLC: t_{R}=4.22 min (A=10 mM NH₄OAc, pH=7.0; B═CH3CN, solvent gradient: 0% B to 50% B in 15 min); Preparative RP-C₁₈ HPLC: t_{R}=11.7 min (A=10 mM NH₄OAc, pH=7.0; B=CH3CN, solvent gradient: 0% B to 50% B in 30 min); λₘₐₓ=280 nm; HPLC(Agilent Preparative C18 Column): Mobile phase: Buffer (pH 7)—CH3CN; Method: 0-30 CH3CN-30 min, t_{R}=11.7 min. LC-MS(Agilent G6130B Quadrupole LC/MS): Mobile phase: Buffer (pH 7)—CH3CN; Method: 0-50 CH3CN-15 min, t_{R}=4.22 min. MS m/z: MS-API: Calcd. for C₃₆H₅₅N₁₀O₁₂ ([M+H]⁺): 819.4, found, 819.2. ¹H NMR (DMSO-d₆/D₂O) δ=8.63 (s, 1H), 7.64 (d, J=8.8 Hz, 2H), 6.64 (d, J=8.8 Hz, 2H), 4.48 (s, 2H), 4.12-4.21 (m, 1H), 3.58 (t, J=6.4 Hz, 2H), 3.41-3.53 (m, 24H), 3.18-3.25 (m, 2H), 3.11-3.18 (m, 2H), 2.28 (t, J=6.4, 2H), 2.15 (t, J=7.4, 2H), 2.03 (m, 1H), 1.88 (m, 1H) ppm.

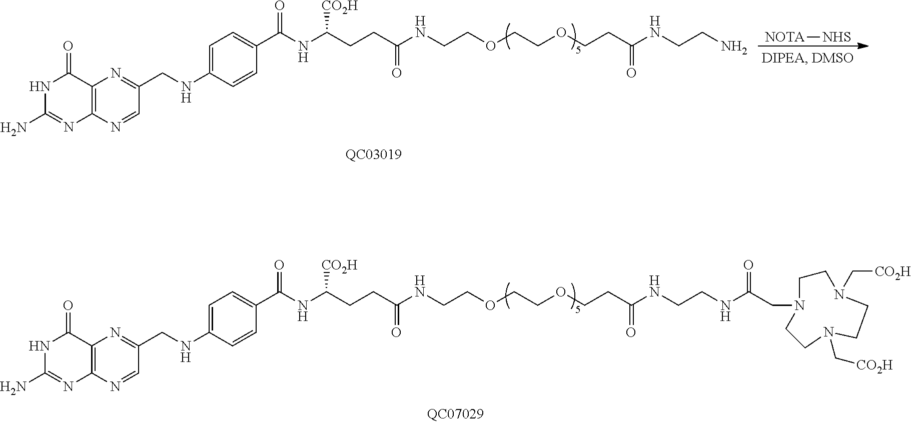

EXAMPLE. FA-PEG₆-NOTA. To QC03019 (9.5 mg, 0.011 mmol) in DMSO (0.40 ml, with a concentration at 0.029 M) was added NOTA-NHS(8.6 mg, 0.013 mmol) followed by DIPEA (7.0 μL, 0.039 mmol). The reaction was stirred at 23° C., monitored by LC-MS, and most of the starting material was transformed to the corresponding product in 5 hours. The crude material was purified by RP-C₁₈ HPLC to afford the pure product QC07029 (5.5 mg, 45%). Analytical RP-C₁₈ HPLC: t_{R}=3.91 min (A=10 mM NH₄OAc, pH=7.0; B=CH3CN, solvent gradient: 0% B to 50% B in 15 min); Preparative RP-C₁₈ HPLC: t_{R}=10.51 min (A=10 mM NH₄OAc, pH=7.0; B=CH3CN, solvent gradient: 0% B to 50% B in 30 min); 2max=280 nm; HPLC(Agilent Preparative C18 Column): Mobile phase: Buffer (pH 7)—CH3CN; Method: 0-CH3CN-30 min, t_{R}=10.51 min. LC-MS(Agilent G6130B Quadrupole LC/MS): Mobile phase: Buffer (pH 7)-ACN; Method: 0-50 ACN-15 min, t_{R}=3.91 min MS m/z: MS-API: Calcd. for C₄₈H₇₄N₁₃O₁₇ ([M+H]⁺): 1104.5, Found: 1104.4

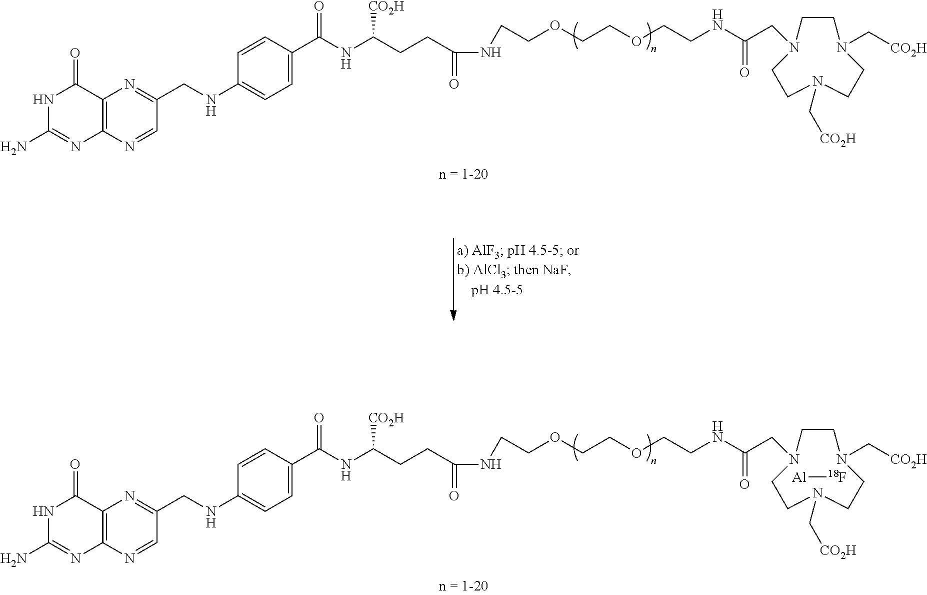

EXAMPLE. FA-NOTA-A¹-¹⁸F Radiotracer [2]. Two methods for the formation of FANOTA-A¹-¹⁸F are described herein. Conditions including the pH value, concentration of the substrates and temperature for the chelating reaction with ¹⁸F—Al can be varied. The general methods for FA-NOTA-A¹-¹⁸F are described as followed:

Method a). FA-NOTA Precursor was dissolved in 2 mM NaOAc (pH 4.5) and 0.5 mL of ethanol, which was treated with Al¹⁸F₃·3H₂O (1.5 eq.) which was freshly prepared before application. The pH was adjusted to 4.5-5.0, and the reaction mixture was refluxed for 15-30 min with pH kept at 4.5-5.0. After being cooled down to room temperature, the crude material was loaded to a cartridge, and the radiotracer was eluted into vial. After sterile filtration and being diluted to appropriate radioactivity (5-10 mCi) and specific activity (>1 Ci/μmol), the radiotracer was ready for in vivo PET imaging study.

Method b). FA-NOTA Precursor was dissolved in 2 mM NaOAc (pH 4.5), and treated with AlCl₃·3H₂O (1.5 eq.). The pH was adjusted to 4.5-5.0, and the reaction mixture was refluxed for 15-30 min with pH kept at 4.5-5.0. The crude material was purified by RP-HPLC to afford the FA-NOTA-Al—OH intermediate ready for ¹⁸F-labeling. Appropriate amount of FA-NOTA-Al—OH was treated with Na¹⁸F saline solution and ethanol (1/1, v/v), and the whole mixture was heated at 100-110° C. for 15 min. After being cooled down to room temperature, the crude material was loaded to a cartridge, and the radiotracer was eluted into vial. After sterile filtration and being diluted to appropriate radioactivity (5-10 mCi) and specific activity (>1 Ci/μmol), the radiotracer was ready for in vivo PET imaging study.

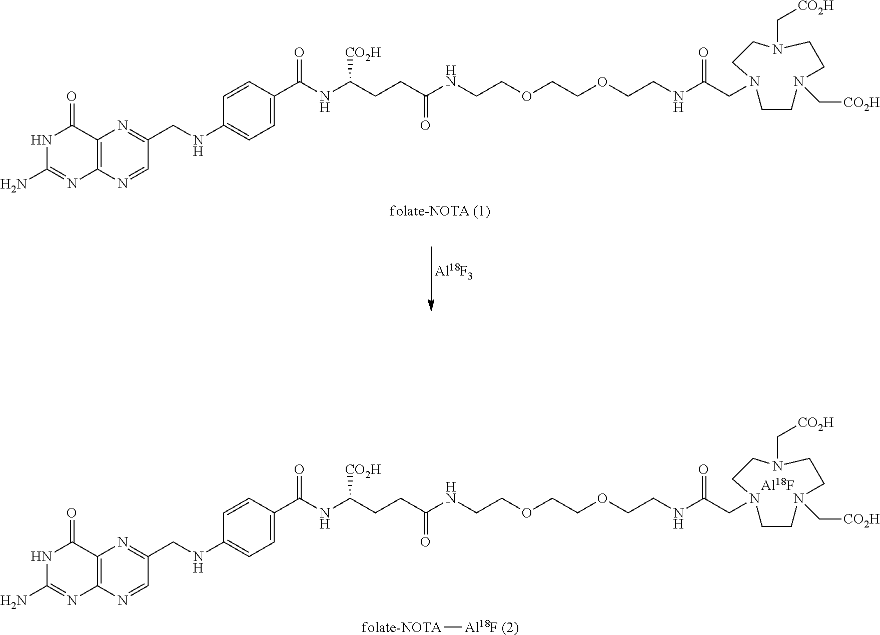

EXAMPLE. Standard Protocol for the Formulation of Folate-NOTA-Al¹⁸F Radiotracer. The resin containing ¹⁸F was first washed with 1.5 mL of ultrapure water, and then ¹⁸F was eluted out from resin by using 1.0 mL of 0.4 M KHCO₃ solution. 100 μL of the eluting solution containing ¹⁸F was added to a stem vial charged with 10 μL acetic acid, 25 μL AlCl3 (2 mM in 0.1 M NaOAc pH 4 buffer) and 125 μL 0.1 M NaOAc pH 4 buffer. The whole mixture was incubated for 2 min before 0.25 mg folate-NOTA precursor (1) in 125 μL of 0.1 M NaOAc pH 4 buffer was transferred to the same stem vial. The reaction was immediately heated to 100° C. for 15 min.

After cooling to room temperature, the crude material was mixed with 0.7 mL 0.1% formic acid and purified by radioactive HPLC on a Xselect CSH C18 (250× 10 mm) column using MeCN and 0.1% formic acid as the mobile phase. The fraction at 11.5 min was collected to afford pure radiotracer in ˜ 40-50% radiochemical yield (RCY) with ˜ 98% radiochemical purity (RCP). The total radiochemical synthesis of folate-NOTA-Al¹⁸F (2, Al¹⁸F-QC07017) was accomplished in ˜37 min with a specific activity (SA) of 70±18.4 GBq/μmol. After sterile filtration and appropriate dilution in isotonic saline to the desired radioactivity, the folate-NOTA-Al¹⁸F (2) radiotracer was ready for PET imaging study.

Using same strategy, radiochemcial synthesis of FA-PEG₁₂-NOTA-A¹-¹⁸F radiotracer (QC07043) was accomplished in ˜35 min with a specific activity (SA) of 49±17.1 GBq/μmol. Although the radiochemical purity is excellent, 100% after radioactive HPLC purification, the total radiochemical yield (RCY) is relatively low, ˜ 25-30%. After sterile filtration and appropriate dilution in isotonic saline to the desired radioactivity, the FA-PEG₁₂-NOTA-A¹-¹⁸F radiotracer was ready for PET imaging study.

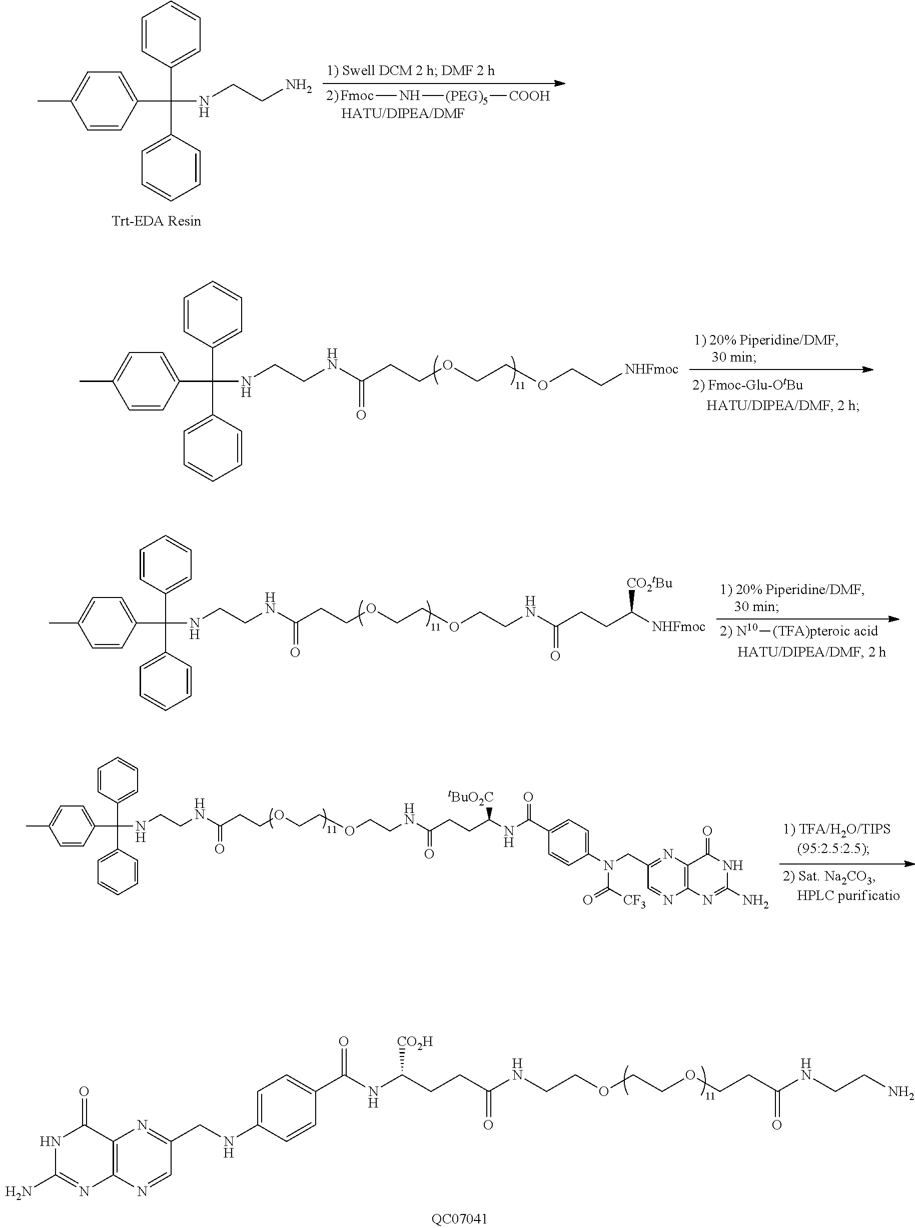

EXAMPLE. Solid Phase Synthesis (SPS) of FA-PEG₁₂-EDA-NH₂ (QC07042) [11]. 1,2-Diaminoethane trityl resin (1.2 mmol/g, 50 mg, 0.06 mmol) was swollen with dichloromethane (DCM, 3 mL) followed by dimethyl formamide (DMF, 3 mL). After swelling the resin in DMF, a solution of fluorenylmethoxycarbonyl (Fmoc)-PEG₁₂-OH(1.5 equiv), HATU (1.5 equiv), and DIPEA (2.0 equiv) in DMF was added. Argon was bubbled for 2 h, and resin was washed with DMF (3×3 mL) and i-PrOH(3×3 mL). The above sequence was repeated for two more coupling steps for conjugation of Fmoc-Glu-(OtBu)-OH and N¹⁰-TFA-Ptc-OH. The final product was cleaved from the resin using a trifluoroacetic acid (TFA): H₂O: triisopropylsilane cocktail (95:2.5:2.5) and concentrated under vacuum. The concentrated product was precipitated in diethyl ether and dried under vacuum, which was then incubated in Sat. Na₂CO₃ and monitored by LC-MS. 1 hour later, the mixture was neutralized to pH=7 with 2 M HCl (aq.) which was purified by preparative with preparative RP-C₁₈ HPLC [solvent gradient: 0% B to 50% B in 30 min; A=10 mM NH₄OAc, pH=7; B=CH3CN]. Acetonitrile was removed under vacuum, and the residue was freeze-dried to yield pure QC07042 as a yellow solid (32.5 mg, 50%). Analytical RP-C₁₈ HPLC: t_{R}=4.76 min (A=10 mM NH₄OAc, pH=7.0; B=CH3CN, solvent gradient: 0% B to 50% B in 15 min); Preparative RP-C₁₈ HPLC: t_{R}=13.75 min (A=10 mM NH₄OAc, pH=7.0; B=CH3CN, solvent gradient: 0% B to 50% B in 30 min); UV-Vis: 2max=280 nm; Preparative RP-C₁₈ HPLC: HPLC(Agilent Preparative C18 Column): Mobile phase: Buffer (pH 7)—CH3CN; Method: 0-50 CH3CN, 30 min, t_{R}=13.75 min. LC-MS: LC-MS(Agilent G6130B Quadrupole LC/MS) of Product Mobile phase: Buffer (pH 7)—CH3CN; Method: 0-50 CH3CN, 15 min, t_{R}=4.76 min. MS m/z: MS-API: Calcd. for C₄₈H₇₉N₁₀O₁₈ ([M+H]⁺): 1083.6, Found: 1083.4;

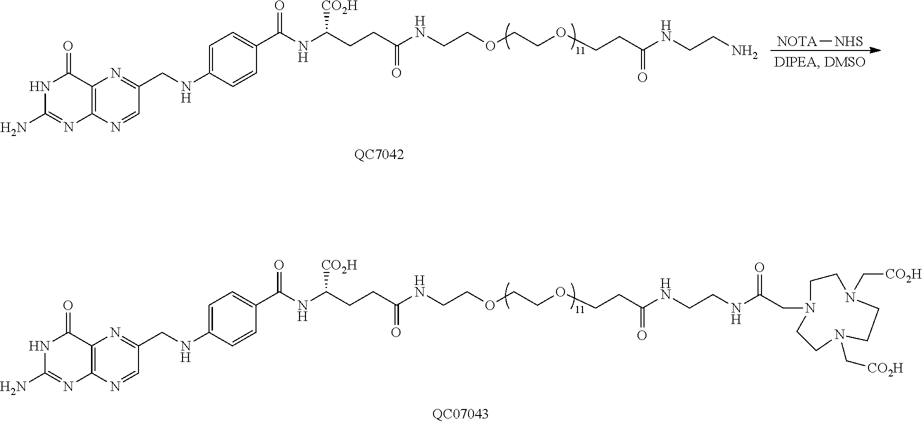

EXAMPLE. FA-PEG₁₂-EDA-NH₂-NOTA (QC07043). To FA-PEG₁₂-EDA-NH₂ (QC07042, 4.78 mg, 0.004 mmol, M.W.: 1082.5) in DMSO (0.25 ml, with a concentration at 0.025 M) was added NOTA-NHS(3.5 mg, 0.005 mmol, 1.2 eq.) followed by DIPEA (2.7 μL, 0.039 mmol). The whole mixture was stirred at 23° C. and monitored by LC-MS. 4 hours later, LC-MS showed that almost all of the starting material was transformed to the product. The crude material was then purified by preparative RP-HPLC to afford the pure FA-PEG₁₂-EDA-NH₂-NOTA (QC07043, 4.09 mg, 68%). Analytical RP-C₁₈ HPLC: t_{R}=6.21 min (A=10 mM NH₄OAc, pH=7.0; B=CH3CN, solvent gradient: 0% B to 30% B in 15 min); Preparative RP-C₁₈ HPLC: t_{R}=15.60 min (A=10 mM NH₄OAc, pH=7.0; B=CH3CN, solvent gradient: 0% B to 30% B in 30 min); UV-Vis: λₘₐₓ=280 nm; LC-MS: LC-MS(Agilent G6130B Quadrupole LC/MS) of Product Mobile phase: Buffer (pH 7)—CH3CN; Method: 0=−8.00 (m, 1H), 7.55 (d, J=6.4 Hz, 1H), 7.54 (s, br, 2H), 6.81-6.93 (m, 1H), 6.62 (d, J=8.0 Hz, 2H), 4.45 (d, J=4.4 Hz, 2H), 3.95-4.03 (m, 1H), 3.64-3.70 (m, 2H), 3.56-3.63 (m, 6H), 3.38-3.50 (m, 28H), 3.33-3.36 (m, 6H), 3.20-3.24 (m, 4H), 3.09-3.18 (m, 10H), 3.04-3.09 (m, 4H), 2.50 (s, 12H, overlapping with the residue peak of DMSO), 2.27-2.34 (m, 2H), 2.02-2.12 (m, 2H), 1.99-2.01 (m, 2H).

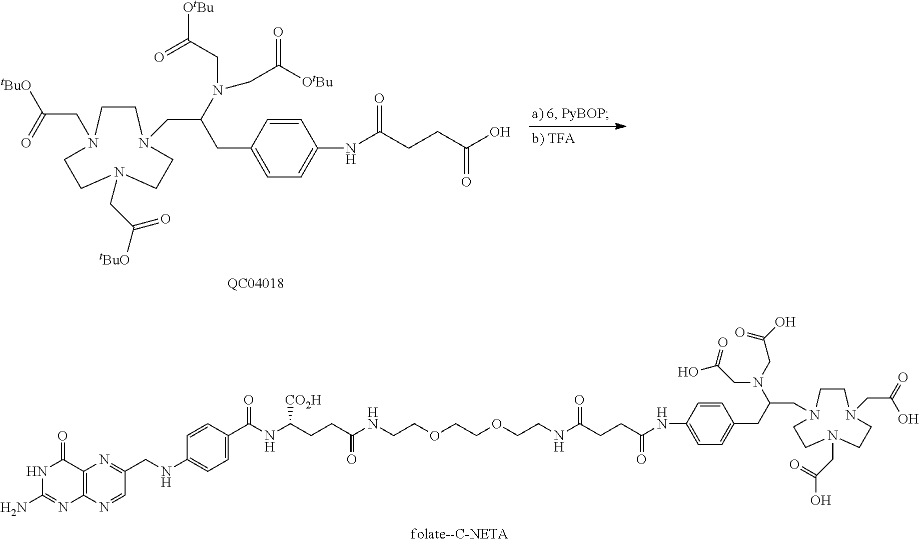 [SMILES: CC(C)(C)OC(=O)CN1CCN(CC(=O)OC(C)(C)C)CCN(CC(Cc2ccc(NC(=O)CCC(=O)O)cc2)N(CC(=O)OC(C)(C)C)CC(=O)OC(C)(C)C)CC1 ; Nc1nc2ncc(CNc3ccc(C(=O)N[C@@H](CCC(=O)NCCOCCOCCNC(=O)CCC(=O)Nc4ccc(CC(CN5CCN(CC(=O)O)CCN(CC(=O)O)CC5)N(CC(=O)O)CC(=O)O)cc4)C(=O)O)cc3)nc2c(=O)[nH]1]

EXAMPLE. C-NETA and folate-C-NETA. A PyBOP promoted coupling between QC04018 and compound 6, followed by deprotection of tert-butyl ester with TFA, provided folate-C-NETA. The folate-C-NETA is used to evaluate the labeling efficiency with Al¹⁸F and ⁶⁸Ga and evaluate the in vivo PET imaging.

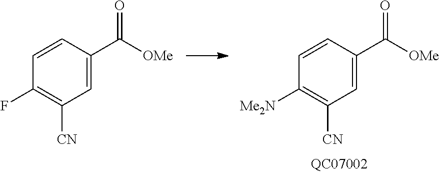 [SMILES: COC(=O)c1ccc(F)c(C#N)c1 ; COC(=O)c1ccc(N(C)C)c(C#N)c1]

EXAMPLE. Methyl 3-cyano-4-(dimethylamino)benzoate (QC07002) [1]. To a stirred solution of methyl 3-cyano-4-fluorobenzoate (5 g, 27.9 mmol) in DMSO (6 ml) was added dimethylamine hydrochloride (2.75 g, 33.7 mmol) followed by potassium carbonate (8.1 g, 58.6 mmol). The reaction mixture was stirred at room temperature overnight and concentrated. The residue was dissolved in dichloromethane (50 ml) and washed with water (2×25 ml), brine, dried over Na₂SO₄ and concentrated in vacuo to give the methyl 3-cyano-4-(dimethylamino)benzoate (QC07002) in quantitative yield and was used without further purification.

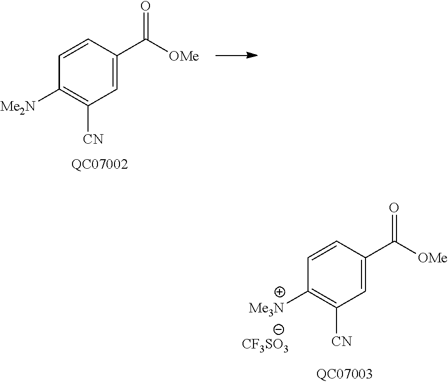

EXAMPLE. 2-Cyano-4-(methoxycarbonyl)-N,N,N-trimethylbenzenaminium trifluoromethanesulfonate (QC07003). To a stirred solution of methyl 3-cyano-4-(dimethylamino)benzoate (3.4 g, 16.7 mmol) in anhydrous dichloromethane (17 ml) was added methyl trifluoromethanesulfonate (10 g, 60.9 mmol, M.W. 164.1) dropwise. The reaction was stirred at RT for 16 h and another portion of methyl trifluoromethanesulfonate (10 g, 60.9 mmol, M.W.: 164.1) was added. The reaction was stirred for another 16 hours and tert-butylmethylether (20 ml) was added slowly. The suspension was filtered and the collected solid was washed with tert-butylmethylether. The crude product was purified by RP-C₁₈ HPLC: (acetonitrile/water-gradient 1:99 to 80:20) to afford product QC07003 (3.69 g) in 60% yield. Analytical RP-C₁₈ HPLC: t_{R}=0.49 min (A=10 mM NH₄OAc, pH=7.0; B=CH3CN, solvent gradient: 0% B to 100% B in 15 min); 2max=275 nm; LC-MS(Agilent G6130B Quadrupole LC/MS): Mobile phase: Buffer (pH 7)—CH3CN; Column: Analytic C₁₈ column; Method: 0-100 CH3CN-15 min, t_{R}=0.49 min. MS m/z: MS-API: Calcd. for C₁₂H₁₅N₂O₂ ([M]⁺): 219.1, Found: 219.0; 1H NMR (400 MHZ, D₂O) δ=8.67 (d, J=2.1 Hz, 1H), 8.44 (dd, J=9.1, 2.1 Hz, 1H), 8.15 (d, J=9.1 Hz, 1H), 3.93 (s, 3H), 3.87 (s, 9H) ppm.

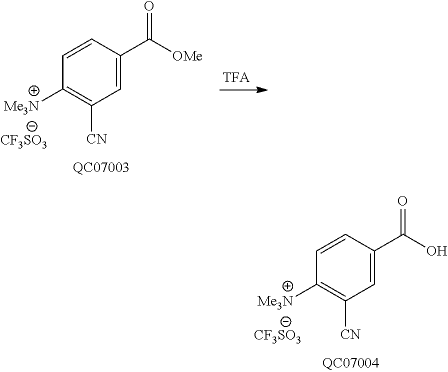

EXAMPLE. 4-Carboxy-2-cyano-N,N,N-trimethylbenzenaminium trifluoromethanesulfonate (QC07004). A solution of QC07003 (3.6 g, 9.8 mmol) in water (83 ml) and TFA (83 ml) was heated at 120° C. for 48 h. The reaction mixture was concentrated in vacuo, the light green oil was treated with diethylether to result a suspension. This solid was collected by filtration, washed with diethylether and dried in vacuo to give 4-carboxy-2-cyano-N,N,Ntrimethylbenzenaminium trifluoromethanesulfonate QC07004 (2.8 g, 82%). Analytical RP-C₁₈ HPLC: t_{R}=0.61 min (A=10 mM NH₄OAc, pH=7.0; B=CH3CN, solvent gradient: 0% B to 100% B in 15 min); 2max=240 nm. LC-MS(Agilent G6130B Quadrupole LC/MS): Mobile phase: Buffer (pH 7)—CH3CN; Column: Analytic C₁₈ column; Method: 0-100 CH3CN 15 min, t_{R}=0.61 min. MS m/z: MS-API: Calcd. for C₁₁H₁₃N₂O₂ ([M]⁺): 205.1, Found: 205.1; 1H NMR (400 MHZ, DMSO) δ=8.58 (d, J=2.07 Hz, 1H), 8.39-8.49 (m, 1H), 8.23-8.35 (m, 1H), 3.85 (s, 9H).

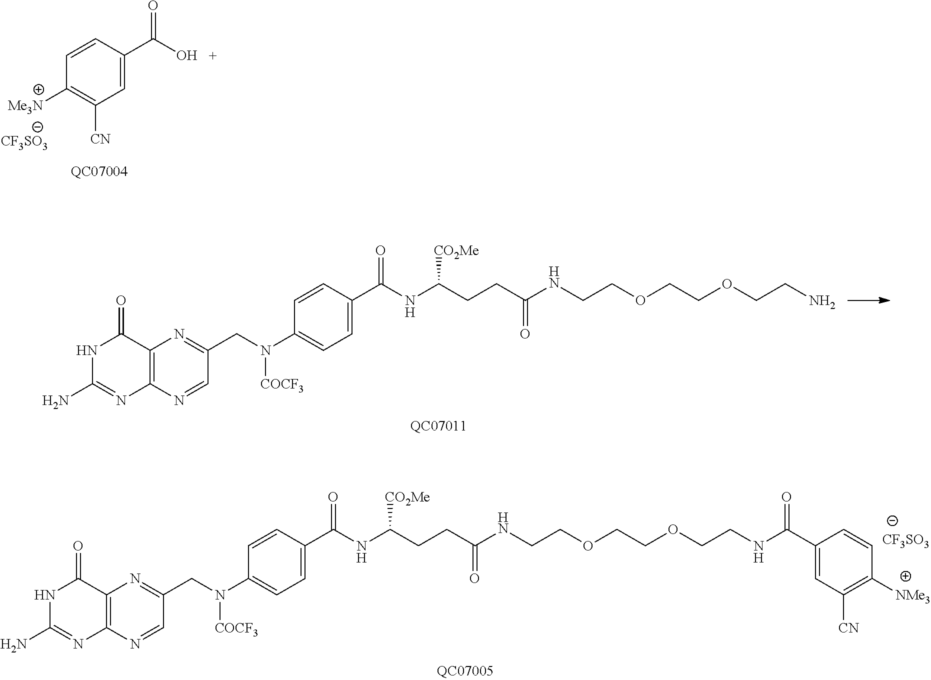

EXAMPLE. FA-PEG1-TMA Precursor (QC07005). QC07004 (62 mg, 0.17 mmol) was added to the solution of QC07011 (0.14 mmol) and DIPEA (87 μL, 1.75 mmol) in DMSO (2.0 mL) at 23° C. under N₂. After being stirred for 15 min at 23° C., PyBOP (91 mg, 0.17 mmol) was added, and the reaction mixture was stirred for 24 h at 23° C. Volatile material was removed under reduced vacuum to afford the crude product which was further purified by RP-HPLC(C₁₈) to afford the pure compound QC07005 as pale yellow colored solid (125.1 mg, 72%). Analytical RP-C₁₈ HPLC: t_{R}=4.17 min (A=10 mM NH₄OAc, pH=7.0; B=CH3CN, solvent gradient: 0% B to 100% B in 15 min); 2max=280 nm; LC-MS(Agilent G6130B Quadrupole LC/MS): Mobile phase: Buffer (pH 7)—CH3CN; Column: Analytic C₁₈ column; Method: 0-100 CH3CN-15 min, t_{R}=4.17 min. MS m/z: MS-API: Calcd. for C₂₈H₃₅F₃N₉O₈ ([M]⁺): 868.3, Found: 868.2.

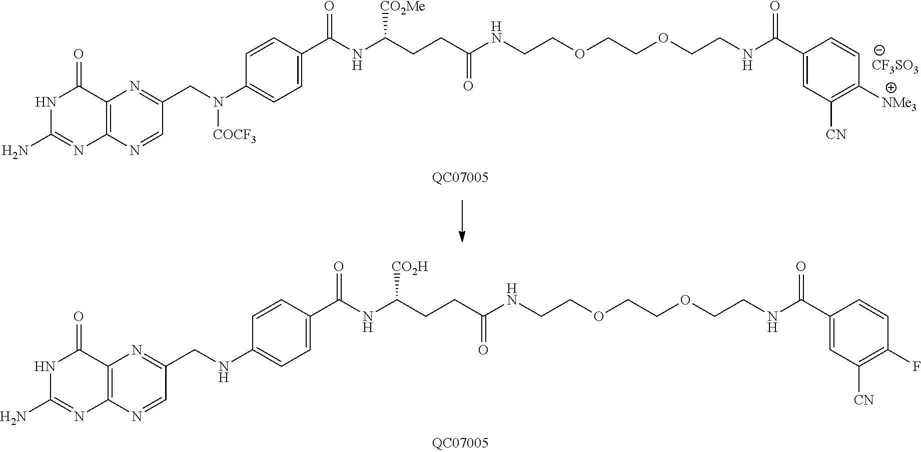

EXAMPLE. General procedure for the one-pot ¹⁹F-introduction and deprotection. 8.3 μL of freshly prepared KF-Kryptofix (1/1.5)(0.0012 mmol, 0.144 M) solution was azeotropically dried with CH3CN at 90-100° C., to which 1.2 mg (0.0012 mmol, 1.0 equiv.) QC07005 in 50 μl of anhydrous DMSO was added with a concentration of precursor at 0.024 M. The resulting mixture was immediately immersed into an oil bath preheated to 70-75° C. and kept at 70-75° C. for 10 min. After being cooled down to room temperature, 200 μl of 1M NaOH(aq.) was added with a concentration of NaOH(aq.) at 0.8 M. The reaction was monitored by LC-MS and found complete after 5 min, which was neutralized with 1 M HCl (aq.) and analyzed by LC-MS(QC07006). And the total labeling efficiency was about 30% based on the analysis of LC-MS. Analytical RP-C₁₈ HPLC: A=10 mM NH₄OAc, pH=7.0; B=CH3CN, solvent gradient: 0% B to 100% B in 15 min; 2max=280 nm; LC-MS: Method: 0-100 CH3CN-15 min, t_{R}=5.13 min. MS m/z: MS-API: Calcd. for C₃₆H₃₇F₄N₁₀O₉ ([M+H]⁺): 829.3, Found: 829.1.

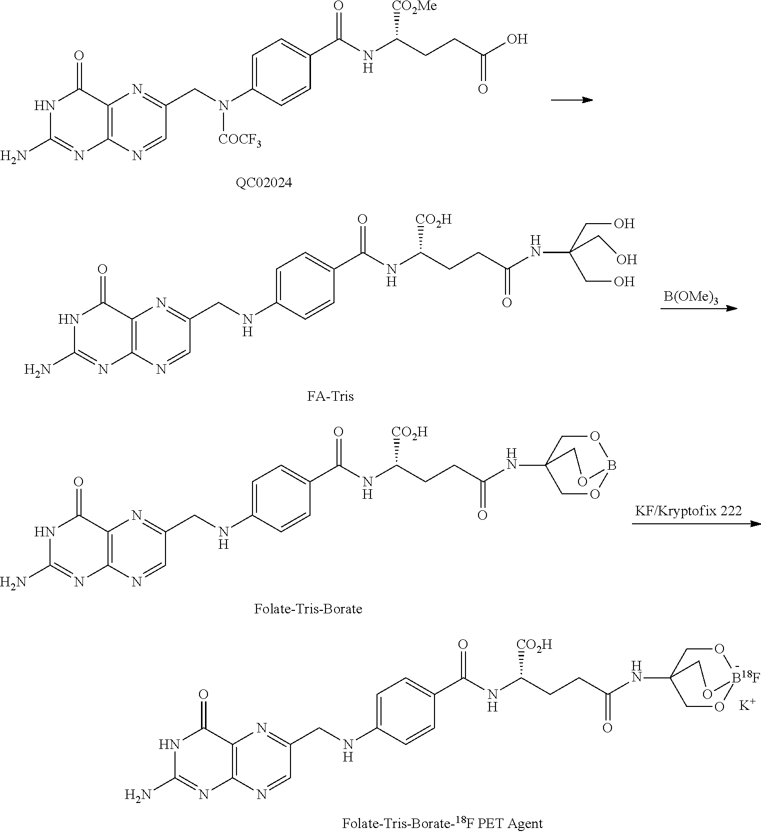

EXAMPLE. Folate-¹⁸F-Boronate PET Imaging Agent

### PSMA Targeted Examples

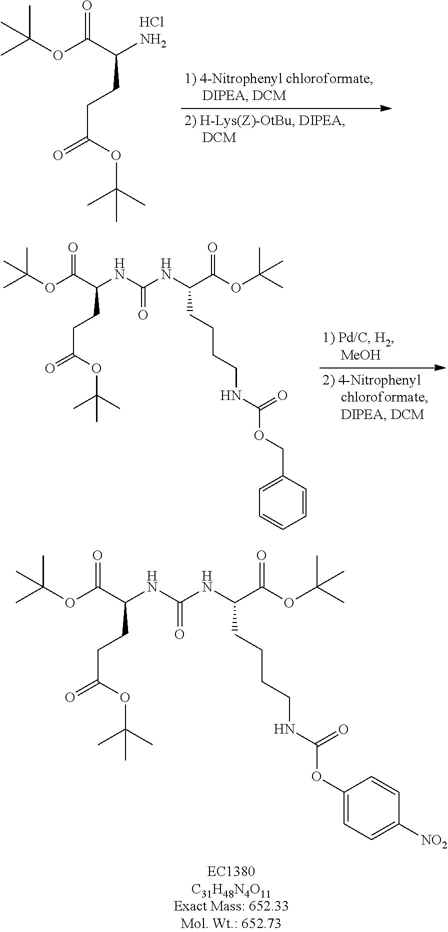

EXAMPLE. EC1380, 10. In a dry flask, H-Glu (O^{t}Bu)-O^{t}Bu·HCl (2.48 g, 8.41 mmol) and 4-nitrophenyl chloroformate (1.86 g, 9.25 mmol, 1.1 eq) were added, dissolved in CH2Cl₂ (30 ml) under Argon atmosphere. The stirring solution was chilled to 0° C., followed by the dropwise addition of DIPEA (4.50 ml, 25.2 mmol, 3 eq). The reaction mixture was allowed to warm to room temperature and stirred for 1 hr. To the stirring solution was added H-Lys-(Z)-O^{t}Bu (4.39 g, 11.8 mmol, 1.4 eq), DIPEA (4.50 ml, 25.2 mmol, 3 eq) and stirred for 1 hr. Upon completion, the reaction was quenched with saturated NaHCO₃ and extracted with CH2Cl₂ three times. The organic extracts were combined, dried over Na₂SO₄, filtered and the solvent was removed via reduced pressure. The product was purified using silica gel chromatography with petroleum ether and ethyl acetate. The Cbz protected amine was transferred to a round bottom flask with 10% Pd/C(10% wt eq), dissolved in MeOH(30 ml) under Hydrogen atmosphere (1atm) and stirred for 3 hr. Upon completion, the reaction mixture was filtered through celite and the solvent was removed via reduced pressure to yield the crude amine. The amine was taken up in CH₂Cl₂ (30 ml) under Argon atmosphere and chilled to 0° C. To the chilled solution was added 4-nitrophenyl chloroformate (2.2 g, 10.9 mmol, 1.3 eq) and DIPEA (6.0 ml, 33.6 mmol, 4 eq) subsequently and stirred for 2 hr at room temperature. The reaction mixture was quenched with saturated NH₄Cl and extract three times with ethyl acetate. The organic extracts were combined, dried over Na₂SO₄, filtered, and solvent was removed under vacuum and purified using silica gel chromatography to yield the desired activated amine, EC1380 (2.54 g, 46%).

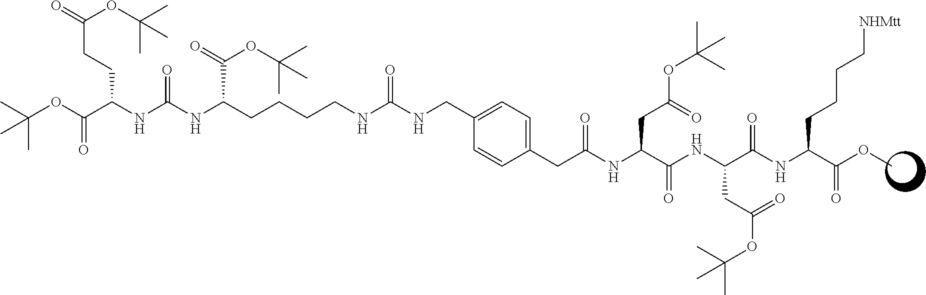

EXAMPLE. Glu (O^{t}Bu)-O^{t}Bu-Lys-O^{t}Bu-AMPAA-Asp (O^{t}Bu)-Asp (O^{t}Bu)-Lys(Mtt)-resin 11. The general procedure described for the synthesis of resin bound folate-peptide resin 1 was followed for the coupling of 2× Fmoc-L-Asp (O^{t}Bu)-OH, Fmoc-AMPAA-OH, Fmoc-L-Lys (Z)-O^{t}Bu, and Fmoc-(L)-Glu (O^{t}Bu) to Fmoc-L-Lys(Mtt)-Wang resin. The resin bound penta-peptide was subjected to standard Fmoc deprotection, washings and Kaiser test. Following another DMF wash (3×10 ml); an EC1380 solution (2.0 eq.) in DMF, and DIPEA (3.0 eq.) were added to the vessel and the solution bubbled with Argon for 2 hour. The coupling solution was filtered, the resin was washed with DMF (3×10 ml) and i-PrOH(3×10 ml) and a Kaiser test was done to assess reaction completion.

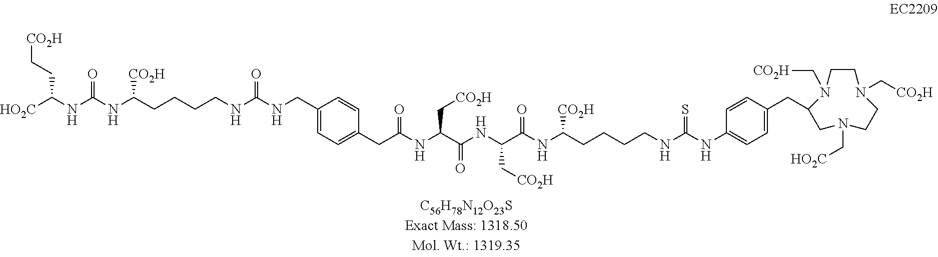 [SMILES: O=C(O)CC[C@H](NC(=O)N[C@@H](CCCCNC(=O)NCc1ccc(CC(=O)N[C@@H](CC(=O)O)C(=O)N[C@@H](CC(=O)O)C(=O)N[C@@H](CCCCNC(=S)Nc2ccc(CC3CN(CC(=O)O)CCN(CC(=O)O)CCN3CC(=O)O)cc2)C(=O)O)cc1)C(=O)O)C(=O)O]

EXAMPLE. Glu-Lys-AMPAA-Asp-Asp-Lys-Bn-NOTA 12. Glu-Lys-AMPAA-Asp-Asp-Lys-Bn-NOTA, EC2209 was prepared in 47% yield according to the process described for folate-peptide-NOTA, 4.¹H NMR (500 MHz DMSO-d₆) Pivotal signals: δ 7.25-7.18 (m, 2H), 7.14 (d, J=8.1 Hz, 1H), 7.12-7.06 (m, 5H), 4.47 (ddd, J=17.8, 7.5, 5.6 Hz, 2H), 4.11-4.08 (m, 3H), 4.08-4.02 (m, 2H), 3.98 (dd, J=8.2, 5.1 Hz, 1H). [M+H]+=Calculated 1319.50, found 1319.70

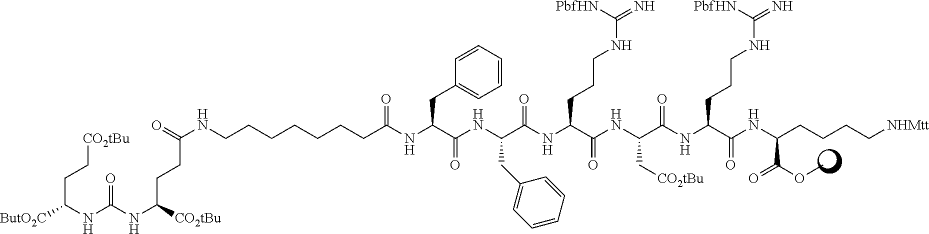

EXAMPLE. Glu (O^{t}Bu)-O^{t}Bu-Lys-O^{t}Bu-Aoc-Phe-Phe-Arg (Pbf)-Asp (O^{t}Bu)-Arg (Pbf)-Lys(Mtt)-resin 13. The general procedure described for the synthesis of resin bound folate-peptide resin 1 was followed for the coupling of Fmoc-L-Arg (Pbf)-OH, Fmoc-L-Asp (O^{t}Bu)-OH, Fmoc-L-Arg (Pbf)-OH, 2× Fmoc-Phe-OH, Fmoc-Aoc-OH, Fmoc-L-Lys (Z)-O^{t}Bu, Fmoc-(L)-Glu (O^{t}Bu) and EC1380 to Fmoc-L-Lys(Mtt)-Wang resin.

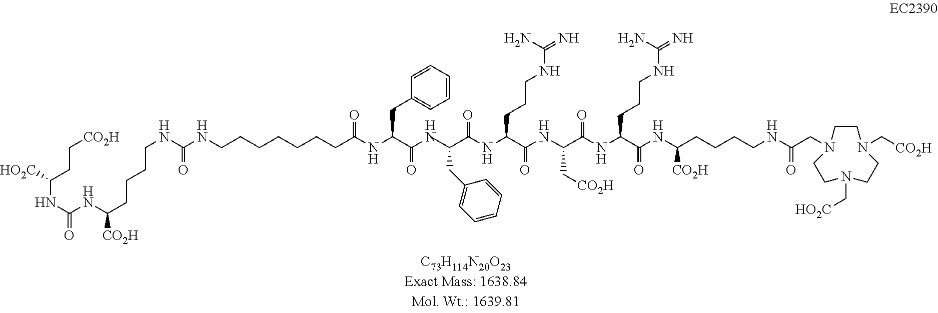

EXAMPLE. Glu-Lys-Aoc-Phe-Phe-Arg-Asp-Arg-Lys-NOTA 14. Glu-Lys-Aoc-Phe-Phe-Arg-Asp-Arg-Lys-NOTA, EC2390 was prepared in 37% yield according to the process described for folate-peptide-NOTA, 4.¹H NMR (500 MHz DMSO-d₆) Pivotal signals: δ 7.25-7.14 (m, 6H), 7.16-7.08 (m, 3H), 4.47 (dd, J=9.0, 4.7 Hz, 1H), 4.42 (t, J=5.9 Hz, 1H), 4.36 (dd, J=10.4, 4.4 Hz, 1H), 4.27 (t, J=6.9 Hz, 1H), 4.16 (t, J=5.6 Hz, 1H), 3.97-3.88 (m, 2H). [M+H]+=Calculated 1639.84, found 1640.22

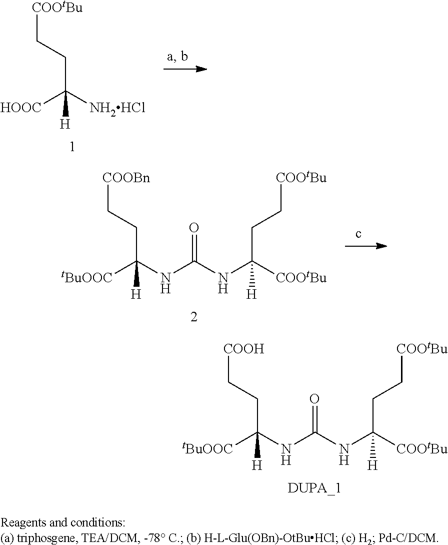

EXAMPLE. DUPA-EAOA-Phe-Arg-Lys-NH₂. 2-[3-(3-Benzyloxycarbonyl-1-tert-butoxycarbonyl-propyl)-ureido]pentanedioic acid di-tert-butyl ester (2). [1, 2] To a solution of L-glutamate di-tert-butyl ester hydrochloride 1 (1.0 g, 3.39 mmol) and triphosgene (329.8 mg, 1.12 mmol) in DCM (25.0 mL) at −78° C., triethylamine (TEA, 1.0 mL, 8.19 mmol) was added. After stirring for 2 h at −78° C. under argon, a solution of L-Glu (OBn)-OtBu (1.2 g, 3.72 mmol) and TEA (600 μL, 4.91 mmol) in DCM (5.0 mL) was added. The reaction mixture was allowed to come to room temperature (rt) over a period of 1 h and stirred at ambient temperature overnight. The reaction was quenched with 1 M HCl, and the organic layer was washed with brine and dried over Na₂SO₄. The crude product was purified using flash chromatography (hexane: EtOAc) 1:1) to yield the intermediate 2 (1.76 g, 90.2%) as a colorless oil and crystallized using hexane: DCM. Rf) 0.67 (hexane: EtOAc) 1:1). ¹H NMR (CDCl₃): § 1.43 (s, 9H, CH3-tBu); 1.44 (s, 9H, CH3-tBu); 1.46 (s, 9H, CH3-tBu); 1.85 (m, 1H, Glu-H); 1.87 (m, 1H, Glu-H); 2.06 (m, 1H, Glu-H); 2.07 (m, 1H, Glu-H); 2.30 (m, 2H, Glu-H); 2.44 (m, 2H, Glu-H); 4.34 [s (broad), 1H, RH]; 4.38 [s (broad), 1H, R—H]; 5.10 (s, 2H, CH2-Ar); 5.22 [s (broad), 2H, Urea-H); 7.34 (m, 5H, Ar—H). EI-HRMS(m/z): (M+H)⁺ calcd for C₃₀H₄₇N₂O₉, 579.3282; found, 579.3289.

EXAMPLE. 2-[3-(1,3-Bis-tert-butoxycarbonyl-propyl)-ureido]pentanedioic Acid 1-tert-Butyl Ester, DUPA_1. To a solution of 2 (250 mg, 432 mmol) in DCM, 10% Pd/C was added. The reaction mixture was hydrogenated at 1 atm for 24 h at rt. Pd/C was filtered through a Celite pad and washed with DCM. The crude product was purified using flash chromatography (hexane: EtOAc) 40:60) to yield DUPA_1 (169 mg, 80.2%) as a colorless oil, and crystallized using hexane: DCM. Rf=0.58 (hexane: EtOAc=40:60). ¹H NMR (CDCl₃): & 1.46 (m, 27H, CH3-tBu); 1.91 (m, 2H,Glu-H); 2.07 (m, 1H, Glu-H); 2.18 (m, 1H, Glu-H); 2.33 (m, 2H, Glu-H); 2.46 (m, 2H, Glu-H); 4.31 (s (broad), 1H, RH); 4.35 (s (broad), 1H, R—H); 5.05 (t, 2H, Urea-H); EI-HRMS(m/z): (M+H)⁺ calcd for C₂₃H₄₁N₂O₉,489.2812; found, 489.2808.

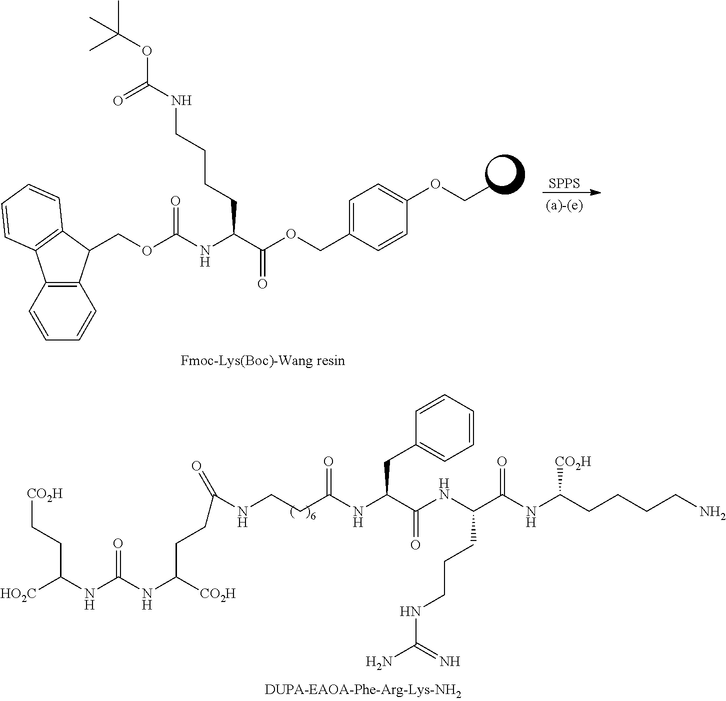

Reagents and conditions: (a)(i) 20% piperidine/DMF,room temperature,10 min; (ii) Fmoc-Arg (Boc) 2-OH, HBTU, HOBt, DMF-DIPEA, 2h. (b)(i) 20% piperidine/DMF,room temperature, 10 min; (ii) Fmoc-Phe-OH, HBTU, HOBt, DMF-DIPEA, 2h. (c)(i) 20% piperidine/DMF, room temperature, 10 min; (ii) Fmoc-8-amino-octanoic (EAO)acid,HBTU,HOBt,DMF/DIPEA,2h. (d)(i) 20% piperidine/DMF, room temperature, 10 min; (ii)(tBuO) 3-DUPA-OH, HBTU, HOBt, DIPEA, 2h. (e) TFA/H₂O/TIPS (95:2.5:2.5), 1h

EXAMPLE. DUPA-EAOA-Phe-Arg-Lys-NH₂. Fmoc-Lys (Boc)-Wang resin (0.43 mM) was swollen with DCM (3 mL) followed by dimethyl formamide (DMF, 3 mL). A solution of 20% piperidine in DMF (3×3 mL) was added to the resin, and argon was bubbled for 5 min. The resin was washed with DMF (3×3 mL) and isopropyl alcohol (i-PrOH, 3×3 mL). Formation of free amine was assessed by the Kaiser test. After swelling the resin in DMF, a solution of Fmoc-Arg (Boc) 2-OH(2.5 equiv), HBTU (2.5 equiv), HOBt (2.5 equiv), and DIPEA (4.0 equiv) in DMF was added. Argon was bubbled for 2 h, and resin was washed with DMF (3× 3 mL) and i-PrOH(3×3 mL). The coupling efficiency was assessed by the Kaiser Test. The above sequence was repeated for 3 more coupling steps to introduce the phenylanaline (Phe), 8-amino-octanoic acid (EAO), and DUPA successively. Final compound was cleaved from the resin using a trifluoroacetic acid (TFA): H₂O: triisopropylsilane cocktail (95:2.5:2.5) and concentrated under vacuum. The concentrated product was precipitated in cold diethyl ether and dried under vacuum. The crude product was purified using preparative RP-HPLC [(2)₂₁₀ nm; solvent gradient: 0% B to 50% B in 30 min run; mobile phase: A) 0.1% TFA, pH=2; B) acetonitrile (ACN)]. ACN was removed under vacuum, and pure fractions were freeze-dried to yield DUPA-EAOA-Phe-Arg-Lys-NH₂ as a white solid. UV/vis: λₘₐₓ=205 nm. Analytical RP-HPLC: t_{R}=6.2 min (A=0.1% TFA; B=CH3CN, solvent gradient: 0% B to 50% B in 15 min); ESI-MS(m/z): (M+H)⁺ calcd for C₄₀H₆₅N₁₀O₁₃, 893.5; found, 893.4.

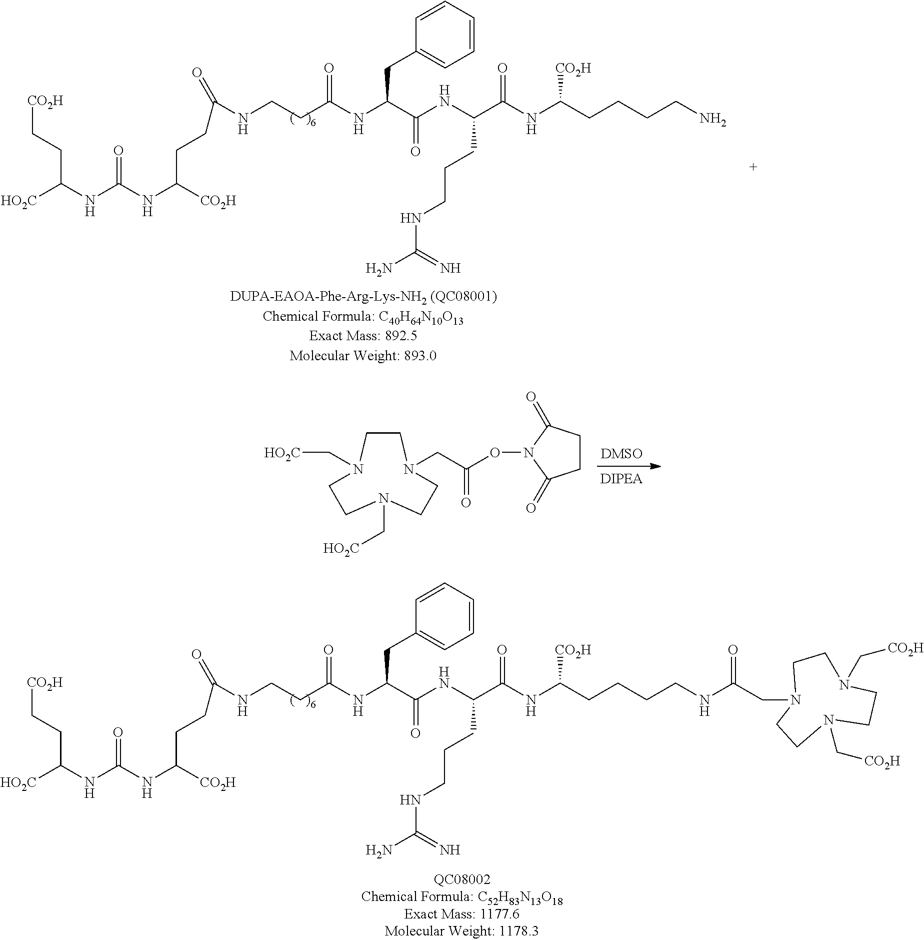

EXAMPLE. DUPA-EAOA-Phe-Arg-Lys-NH₂-NOTA. To DUPA-EAOA-Phe-Arg-Lys-NH₂ (QC08001, 5.0 mg, 0.0056 mmol, M.W.: 893.0) in DMSO (0.20 ml, with a concentration at 0.028 M) was added NOTA-NHS(5.5 mg, 0.0084 mmol, 1.5 eq.) followed by DIPEA (2.9 μL, 0.017 mmol). The reaction was stirred at 23° C., monitored by LC-MS, and most of the starting material was transformed to the corresponding product in 5 hours. The crude material was purified by RP-C₁₈ HPLC. ACN was removed under vacuum, and pure fractions were freeze-dried to yield the pure DUPA-EAOA-Phe-Arg-Lys-NH₂-NOTA (QC08002, 3.3 mg, 50%). Analytical RP-C₁₈ HPLC: t_{R}=5.98 min (A=0.1% TFA; B=CH₃CN, solvent gradient: 0% B to 50% B in 15 min); Preparative RP-C₁₈ HPLC: t_{R}=16.16 min (A=0.1% TFA; B=CH₃CN, solvent gradient: 0% B to 50% B in 30 min); UV-Vis: λₘₐₓ=201 nm; HPLC(Agilent Preparative C₁₈ Column): Mobile phase: A=0.1% TFA; B=CH₃CN; Method: 0-50 CH₃CN-30 min, t_{R}=16.16 min LC-MS(Agilent G6130B Quadrupole LC/MS): Mobile phase: A=0.1% TFA; B=CH₃CN; Method: 0-50 CH₃CN-30 min, t_{R}=5.98 min; MS m/z: MS-API: Calcd. for C₅₂H₈₄N₁₃O₁₈ ([M+H]⁺): 1178.6, Found: 1178.4.

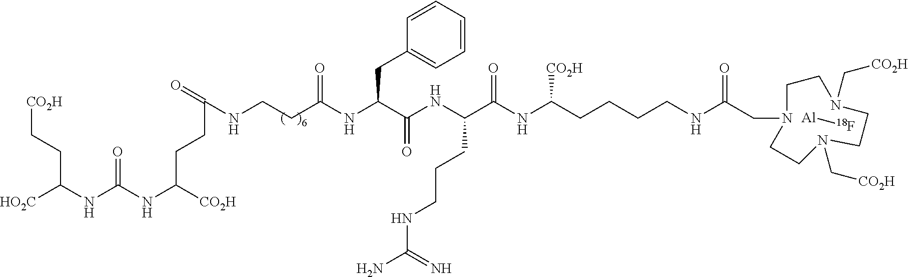

EXAMPLE. DUPA-EAOA-Phe-Arg-Lys-NH₂-NOTA-Al¹⁸F. Method a): . DUPA-EAOA-Phe-Arg-Lys-NH₂-NOTA is dissolved in 2 mM NaOAc (pH 4.5) and 0.5 mL of ethanol, and treated with Al¹⁸F₃·3H₂O (1.5 eq.) which is freshly prepared before application. The pH is adjusted to 4.5-5.0, and the reaction mixture is refluxed for 15-30 min with pH kept at 4.5-5.0. After cooling to room temperature, the crude material is loaded to a cartridge, and the radiotracer eluted into vial. After sterile filtration and being diluted to appropriate radioactivity (5-10 mCi) and specific activity (>1 Ci/μmol), the radiotracer is used in in vivo PET imaging. Method b). DUPA-EAOA-Phe-Arg-Lys-NH₂-NOTA is dissolved in 2 mM NaOAc (pH 4.5), and treated with AlCl3.3H₂O (1.5 eq.). The pH is adjusted to 4.5-5.0, and the reaction mixture is refluxed for 15-30 min with the pH kept at 4.5-5.0. The crude material is purified by RP-HPLC to afford the DUPA-EAOA-Phe-Arg-Lys-NH₂-NOTA-Al—OH intermediate ready for ¹⁸F-labeling. Appropriate amount of DUPA-EAOA-Phe-Arg-Lys-NH₂-NOTA-Al—OH is treated with Na¹⁸F saline solution and ethanol (1/1, v/v), and the whole mixture is heated at 100-110° C. for 15 min. After cooling to room temperature, the crude material is loaded to a cartridge, and the radiotracer eluted into vial. After sterile filtration and being diluted to appropriate radioactivity (5-10 mCi) and specific activity (>1 Ci/μmol), the radiotracer is ready for use in in vivo PET imaging.

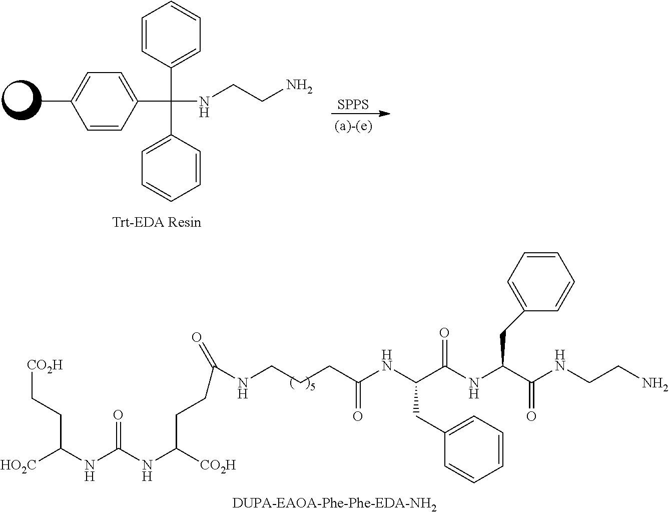

Reagents and conditions: (a) Fmoc-Phe-OH, HBTU, HOBt, DMF/DIPEA, 2h. (b)(i) 20% piperidine/DMF,room temperature, 10 min; (ii) Fmoc-Phe-OH, HBTU, HOBt, DMF/DIPEA, 2h. (c)(i) 20% piperidine/DMF,room temperature, 10 min; (ii) Fmoc-8-amino-octanoic (EAO) acid, HBTU, HOBt, DMF/DIPEA, 2h. (d)(i) 20% piperidine/DMF, room temperature, 10 min; (ii)(tBuO) 3-DUPA-OH, HBTU, HOBt, DIPEA, 2h. (e) TFA/H₂O/TIPS(95:2.5:2.5), 1h.

EXAMPLE. Solid Phase Peptide Synthesis (SPPS) of DUPA-EAOA-Phe-Phe-EDA-NH₂. [2, 3]. As described herein for DUPA-EAOA-Phe-Arg-Lys-NH₂ (QC08001), DUPA-EAOA-Phe-Phe-EDA-NH₂ is prepared. The commercially available Trt-EDA resin was swollen with DCM (3 mL) followed by dimethyl formamide (DMF, 3 mL), to which a solution of Fmoc-Phe-OH(2.5 equiv), HBTU (2.5 equiv), HOBt (2.5 equiv), and DIPEA (4.0 equiv) in DMF was added. Argon was bubbled for 2 h, and resin was washed with DMF (3×3 mL) and i-PrOH(3×3 mL). The coupling efficiency was assessed by the Kaiser Test. A solution of 20% piperidine in DMF (3×3 mL) was added to the resin, and argon was bubbled for 5 min. The resin was washed with DMF (3×3 mL) and isopropyl alcohol (i-PrOH, 3× 3 mL). Formation of free amine was assessed by the Kaiser test. The above sequence was repeated for 3 more coupling steps to introduce the second phenylanaline (Phe), 8-amino-octanoic acid (EAO), and DUPA successively. Final compound was cleaved from the resin using a trifluoroacetic acid (TFA): H₂O: triisopropylsilane cocktail (95:2.5:2.5) and concentrated under vacuum. The concentrated product was precipitated in cold diethyl ether and dried under vacuum. The crude product was purified using preparative RP-HPLC [1] 210 nm; solvent gradient: 0% B to 100% B in 30 min run; mobile phase: A) 10 mM NH₄OAc (pH=7, buffer); B) acetonitrile (ACN)]. ACN was removed under vacuum, and pure fractions were freeze-dried to yield DUPA-EAOA-Phe-Phe-EDA-NH₂ as a white solid. Analytical RP-C₁₈ HPLC: t_{R}=3.99 min (A=10 mM NH₄OAc, pH=7.0; B=CH₃CN, solvent gradient: 0% B to 100% B in 15 min); Preparative RP-C₁₈ HPLC: t_{R}=16.05 min (A=10 mM NH₄OAc, pH=7.0; B=CH₃CN, solvent gradient: 0% B to 100% B in 30 min); UV-Vis: λₘₐₓ=209 nm; LC-MS: LC-MS(Agilent G6130B Quadrupole LC/MS) of Product Mobile phase: Buffer (pH 7)—CH₃CN; Method: 0-100 ACN-15 min, t_{R}=3.99 min. MS m/z: MS-API: Calcd. for C₃₉H₅₆N₇O₁₁ ([M+H]⁺): 798.4, Found: 798.3; Calcd. for C₃₉H₅₅N₇O₁₁K ([M+K]⁺): 836.4, Found: 836.3. HPLC(Agilent Preparative C₁₈ Column): Mobile phase: Buffer (pH 7)—CH₃CN; Method: 0-100 ACN-30 min, t_{R}=16.05 min.

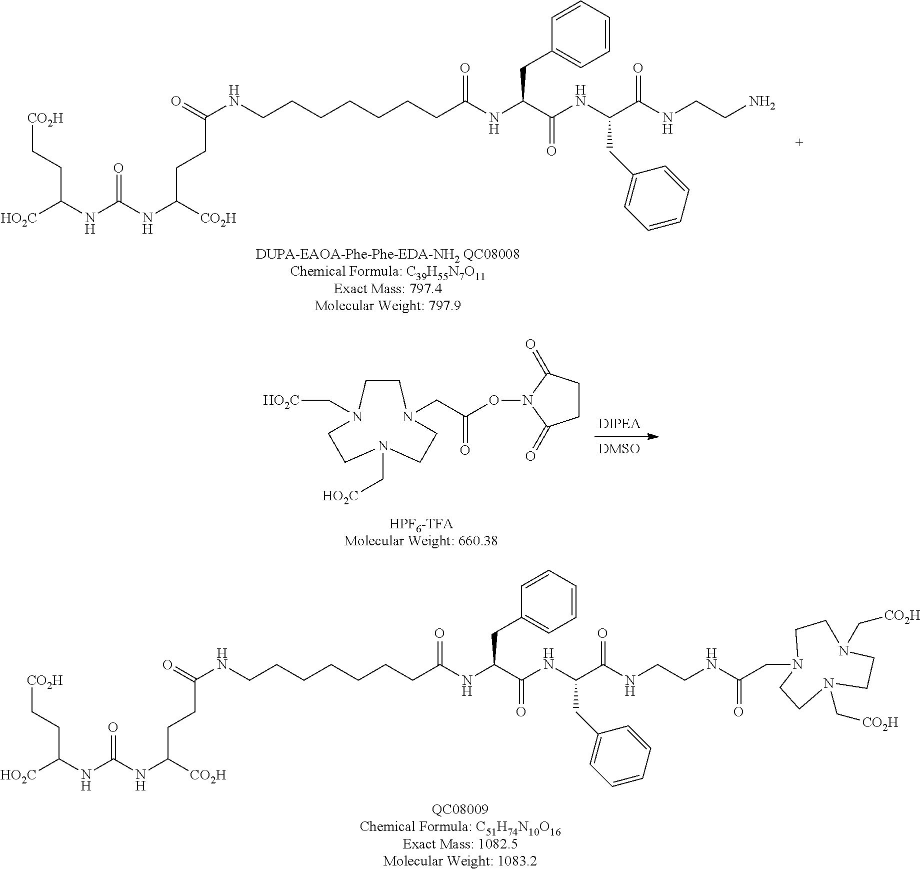 [SMILES: NCCNC(=O)[C@H](Cc1ccccc1)NC(=O)[C@H](Cc1ccccc1)NC(=O)CCCCCCCNC(=O)CCC(NC(=O)NC(CCC(=O)O)C(=O)O)C(=O)O ; O=C(O)CN1CCN(CC(=O)O)CCN(CC(=O)ON2C(=O)CCC2=O)CC1 ; O=C(O)CCC(NC(=O)NC(CCC(=O)NCCCCCCCC(=O)N[C@@H](Cc1ccccc1)C(=O)N[C@@H](Cc1ccccc1)C(=O)NCCNC(=O)CN1CCN(CC(=O)O)CCN(CC(=O)O)CC1)C(=O)O)C(=O)O]

EXAMPLE. To DUPA-EAOA-Phe-Phe-EDA-NH₂ (QC08008, 5.9 mg, 0.0074 mmol, M.W.: 797.4) in DMSO (0.25 ml, with a concentration at 0.025 M) was added NOTA-NHS(7.3 mg, 0.011 mmol, 1.5 eq.) followed by 4 drops of DIPEA. The mixture was stirred at 23° C. and monitored by LC-MS. 4 hours later, LC-MS showed that almost all of the starting material was transformed to the product. The crude material was then purified by preparative RP-HPLC to afford the pure DUPA-EAOA-Phe-Phe-NOTA (QC08009, 4.50 mg, 56%, based on 8.02 mg in theory, 97% purity by HPLC at 210 nm). Analytical RP-C₁₈ HPLC: t_{R}=3.45 min (A=10 mM NH₄OAc, pH=7.0; B=CH₃CN, solvent gradient: 0% B to 100% B in 15 min); Preparative RP-C₁₈ HPLC: t_{R}=10.09 min (A=10 mM NH₄OAc, pH=7.0; B=CH₃CN, solvent gradient: 0% B to 100% B in 30 min); UV-Vis: λmax=211 nm; LC-MS: LC-MS(Agilent G6130B Quadrupole LC/MS) of Product Mobile phase: Buffer (pH 7)—CH₃CN; Method: 0-100 ACN-15 min, t_{R}=3.45 min. MS m/z: MS-API: Calcd. for C₅₁H₇₅N₁₀O₁₆ ([M+H]⁺): 1083.5, Found: 1083.3; HPLC(Agilent Preparative C₁₈ Column): Mobile phase: Buffer (pH 7)—CH₃CN; Method: 0-100 ACN-30 min, t_{R}=10.09 min. ¹H NMR (400 MHZ, DMSO-d₆) δ=10.13 (br, 1H), 8.98 (br, 1H), 8.43 (br, 1H), 7.90 (br, 3H), 7.30-7.10 (m, 10H), 6.37 (br, 1H), 6.28 (br, 1H), 4.60-4.52 (m, 1H), 4.32-4.44 (m, 1H), 4.24-4.31 (m, 2H), 3.95-4.03 (m, 2H), 3.85-3.92 (m, 2H), 3.28 (s, 4H), 3.25 (s, 2H), 3.09 (m, 1H), 3.05 (m, 1H), 2.92-3.02 (m, 4H), 2.54-2.67 (m, 12H), 2.31-2.38 (m, 2H), 2.19-2.31 (m, 3H), 2.11-2.18 (m, 2H), 2.02-2.10 (m, 3H), 1.52-1.72 (m, 4H), 1.25-1.37 (m, 4H), 1.05-1.13 (m, 2H),

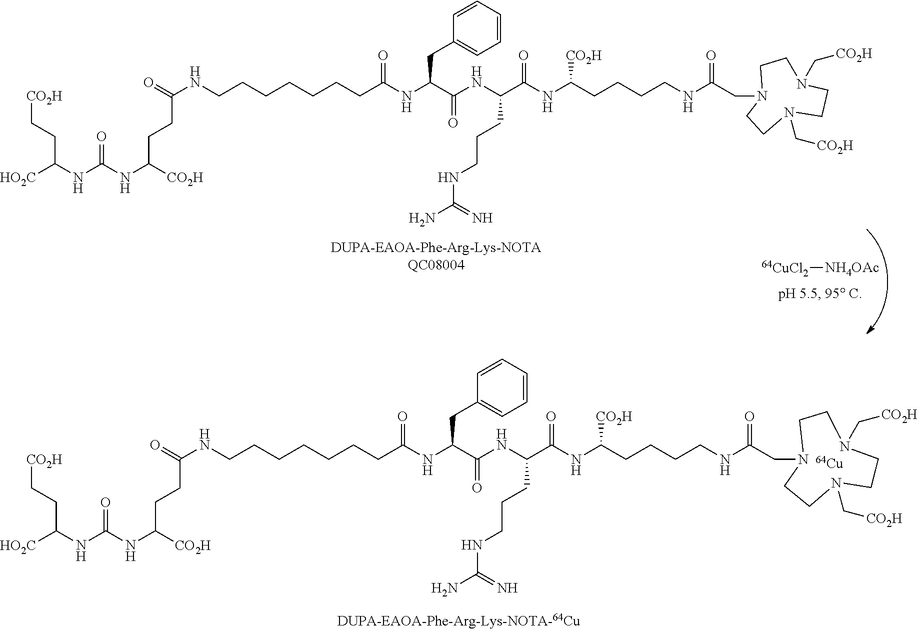

EXAMPLE. Radiochemical Synthesis of DUPA-EAOA-Phe-Arg-Lys-NOTA-⁶⁴Cu Radiotracer. NOTA based chelators have also been reported and employed in the formulation of NOTA-^{64/67}Cu for nuclear medicine/radiotherapy. [14-16] The corresponding DUPA-NOTA-⁶⁴Cu was prepare for the dual purpose of imaging and therapy, also referred to as theranostics. DUPA-EAOA-Phe-Arg-Lys-NOTA-⁶⁴Cu was prepared according a standard protocol with minor modifications. [4, 14-16] The ⁶⁴Cu(OAc) 2, in situ prepared from ⁶⁴CuCl₂ with 0.1 M ammonium acetate (pH 5.5), was added to the reaction tube containing the DUPA-NOTA precursor. The resulting mixture was then heated to 95° C. for 15 min. After cooling to room temperature, the crude material was purified by radioactive HPLC on a C₁₈ column using MeCN and 0.1% TFA as the mobile phase to afford the target radiotracer with ˜ 90% radiochemical purity (RCP). Sterile filtration and dilution in isotonic saline to the desired radioactivity provided the radiotracer ready for PET imaging.

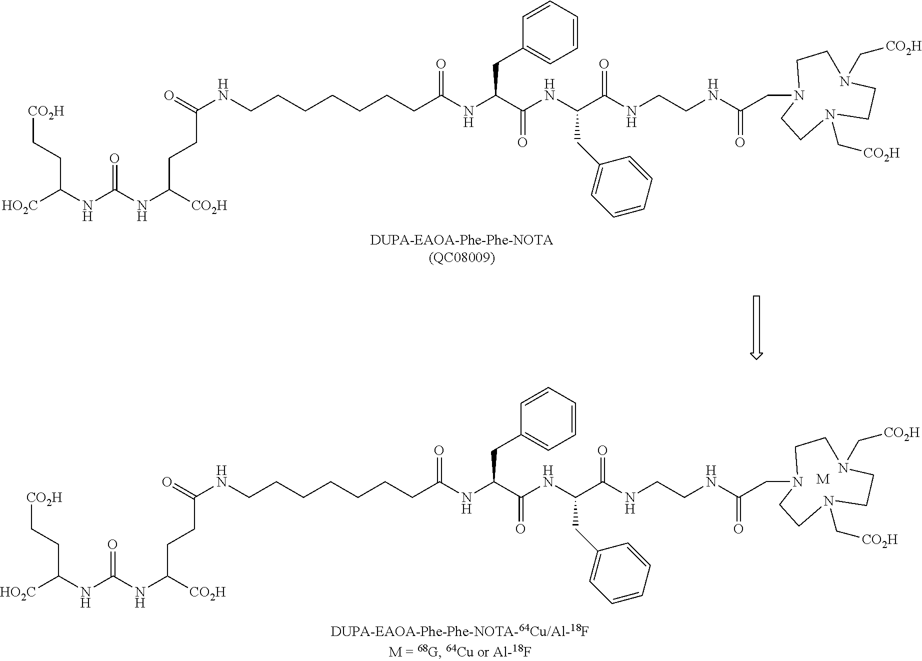

EXAMPLE. Radiochemical synthesis of DUPA-EAOA-Phe-Phe-NOTA-⁶⁴Cu/A¹-¹⁸F.

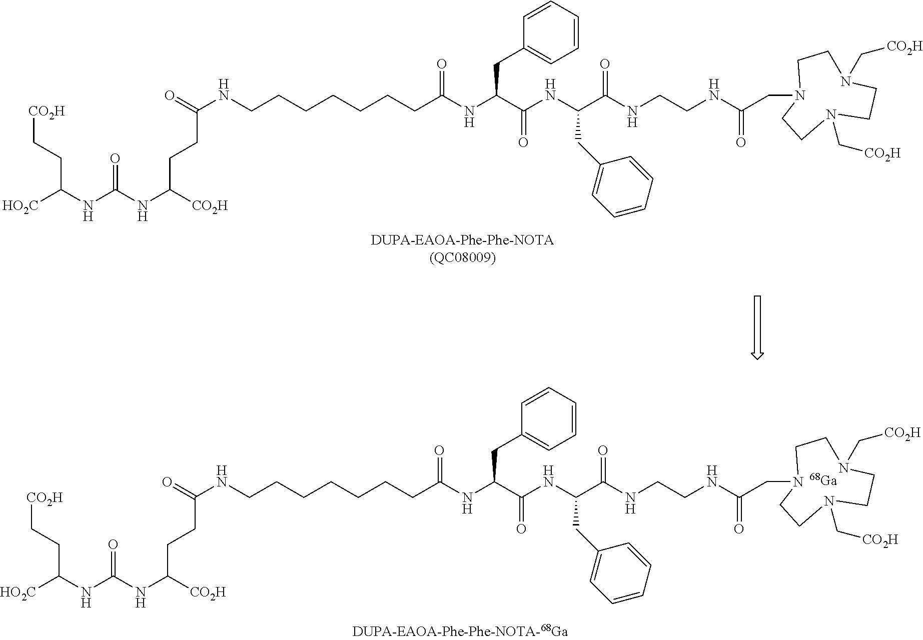

EXAMPLE. Radiochemical synthesis of DUPA-EAOA-Phe-Phe-NOTA-⁶⁸Ga.

EXAMPLE. General procedure for ⁶⁸Ga labeling: ⁶⁸Ga was eluted from the 68Ge/⁶⁸Ga generator with 0.1N HCl. A predetermined amount of ⁶⁸Ga in 0.1N HCl was added to a DUPA-NOTA solution in acetate buffer (pH 4.8). The labeling mixture was incubated at room temperature, and labeling efficiencies were checked by radioactive HPLC. The radiolabeled product was purified by radioactive HPLC and the DUPA-NOTA-⁶⁸Ga peak sample was collected. After sterile filtration and being diluted to appropriate radioactivity (5-10 mCi) and specific activity (>1 Ci/μmol), the radiotracer was ready for in vivo PET imaging study.

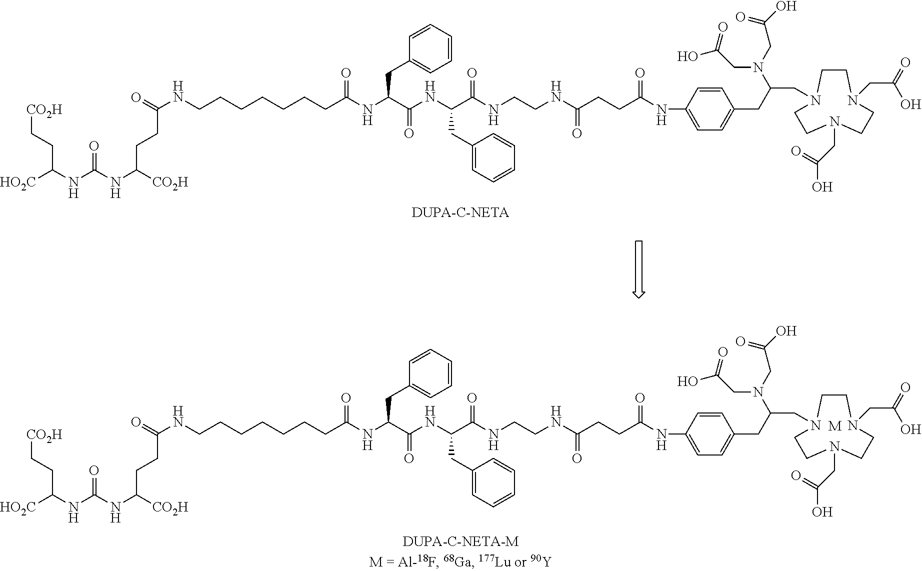

EXAMPLE. Radiochemical synthesis of DUPA-C-NETA based theranostics.

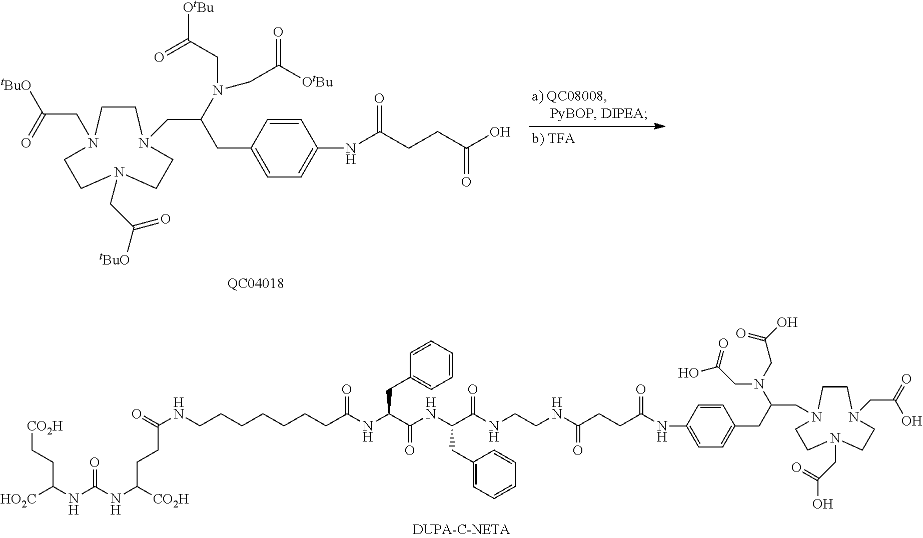 [SMILES: CC(C)(C)OC(=O)CN1CCN(CC(=O)OC(C)(C)C)CCN(CC(Cc2ccc(NC(=O)CCC(=O)O)cc2)N(CC(=O)OC(C)(C)C)CC(=O)OC(C)(C)C)CC1 ; O=C(O)CCC(NC(=O)NC(CCC(=O)NCCCCCCCC(=O)N[C@@H](Cc1ccccc1)C(=O)N[C@@H](Cc1ccccc1)C(=O)NCCNC(=O)CCC(=O)Nc1ccc(CC(CN2CCN(CC(=O)O)CCN(CC(=O)O)CC2)N(CC(=O)O)CC(=O)O)cc1)C(=O)O)C(=O)O]

EXAMPLE. Preparation of the NOTA Derivatives. Bifunctional conjugates, also referred to as theranotics, are described herein. Compounds described herein can tightly chelate both radionuclides such as ¹⁸F and ⁶⁸Ga for PET imaging, and radionuclides ¹⁷⁷Lu and ⁹⁰Y for radiotherapy. C-NETA, a NOTA derivative, has been reported to chelate Al¹⁸F with about twice the efficiency (87%) of NOTA. [17] Moreover, C-NETA also reportedly chelates the commonly used radiotherapeutic nuclides, such as ¹⁷⁷Lu and ⁹⁰Y, with high labeling efficiency. [18] Thus, it is appreciated herein, that C-NETA is useful as a bifunctional chelator that can be used for both PET imaging and radiotherapy, where the radionuclide is a metal or metal halide, such as Al¹⁸F, ⁶⁸Ga, ¹⁷⁷Lu or ⁹⁰Y.

EXAMPLE. A PyBOP promoted coupling between QC04018 and QC08008, followed by deprotection of tert-butyl ester with TFA provides DUPA-C-NETA. DUPA-C-NETA is used to evaluate the labeling efficiency to Al¹⁸F, ⁶⁸Ga, ¹⁷⁷Lu and ⁹⁰Y, and evaluate the in vivo PET imaging and radiotherapy.

### METHOD EXAMPLES

EXAMPLE. The specificity of the radionuclide containing conjugates binding to FR is evaluated against KB xenografts homogenates and Cal51 xenografts homogenates. Concentration dependent binding was evaluated for ¹⁸F-AIF-QC07017 and ¹⁸F-AIF-QC07043, and separated into specific and non-specific binding. Significant non-specific binding was not observed in KB homogenates. Minor non-specific binding was observed in Cal51 homogenates, with a specific/non-specific binding ratio of >3:1 at all concentrations up to about 30 nM for ¹⁸F-AIF-QC07017, and a specific/non-specific binding ratio of >2:1 at all concentrations up to about nM for ¹⁸F-AIF-QC07043. Minor non-specific binding was observed in A549 homogenates, with a specific/non-specific binding ratio of >2:1 at all concentrations up to about 10 nM for ¹⁸F-AIF-QC07043. Scatchard analyses were also performed. Displaceable and saturable binding of ¹⁸F-AIF-QC07017 in human tumor xenografts (KB and Cal51) by self competition was observed. Both ¹⁸F-AIF-QC07017 and ¹⁸F-AIF-QC07043 bound one site with high affinity in all cell xenografts. The high ratio of Bmax/Kd indicated a high specific binding affinity to KB xenografts. Moderate binding affinity was observed for Cal51 xenografts, and the lowest binding affinity was observed for A549 xenografts. Without being bound by theory, it is believed herein that the moderate expression of FR in Cal51 xenografts accounts for the lower binding affinity. Binding affinities of ¹⁸F-AIF-QC07017 (2) to FR in KB and Cal51 tumor crude homogenate.

| Folate-NOTA-Al18F (2) | Bmax, nM* | Kd, nM | Bmax/Kd |
|---|---|---|---|
| KB xenografts | 511 | 0.7 | >700 |
| Cal51 xenografts | 36 | 1.1 | >30 |

Binding affinities of ¹⁸F-AIF-QC07043 to FR in KB and Cal51 tumor crude homogenate.

| FA-PEG12-NOTA-Al18F | Bmax, nM* | Kd, nM | Bmax/Kd |
|---|---|---|---|
| KB xenografts | 241 | 0.4 | 603 |
| Cal51 xenografts | 13 | 1.2 | 11 |

EXAMPLE. μPET imaging was performed on nude mice bearing KB tumor xenografts under baseline and competed conditions to evaluate the in vivo binding specificity of ¹⁸F-AIF-QC07017 (2) to FR. Nude mice bearing KB tumor xenografts on their left shoulder were injected with 0.30-0.40 mCi (2). The competed group received 100 μg of folic acid 10 min before the i.v. injection of (2), and the treatment group was injected with a corresponding volume of phosphate buffer. Time course inspection of PET images obtained at various time points revealed that the data acquired in 60-90 min post tracer injection gave the best visual PET imaging. The KB tumors were clearly visualized in the treated group, whereus the uptake of (2) was completely inhibited by competing with folic acid, supporting a high specificity of (2) binding to FR in vivo. Without being bound by theory, it is believed herein that the high radioactivity found in kidneys was due to the uptake mediated by FR that is expressed in the proximal tubule cells in kidneys and the potential accumulation of radiotracer via renal excretion, which was further supported by the biodistribution studies described herein. With the exception of the liver, significant uptake in other organs was not observed. A significant blocking effect in liver uptake was observed in under competed conditions.

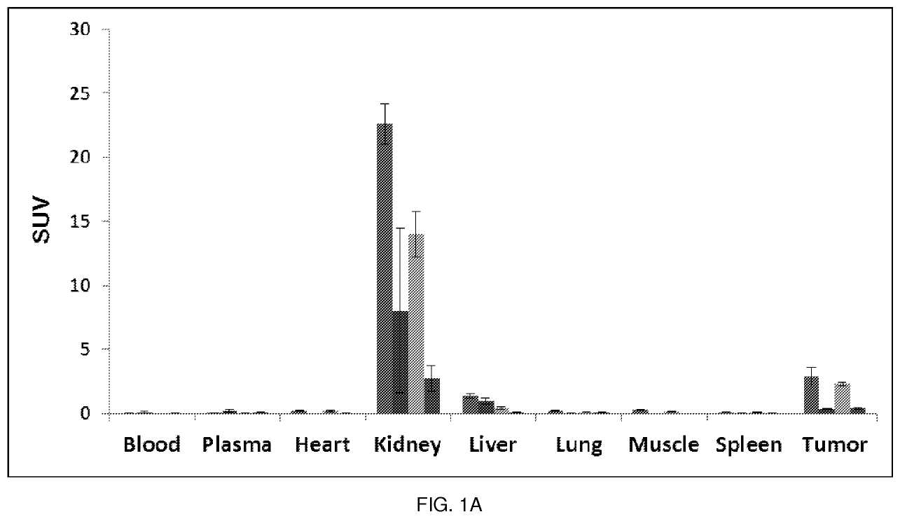

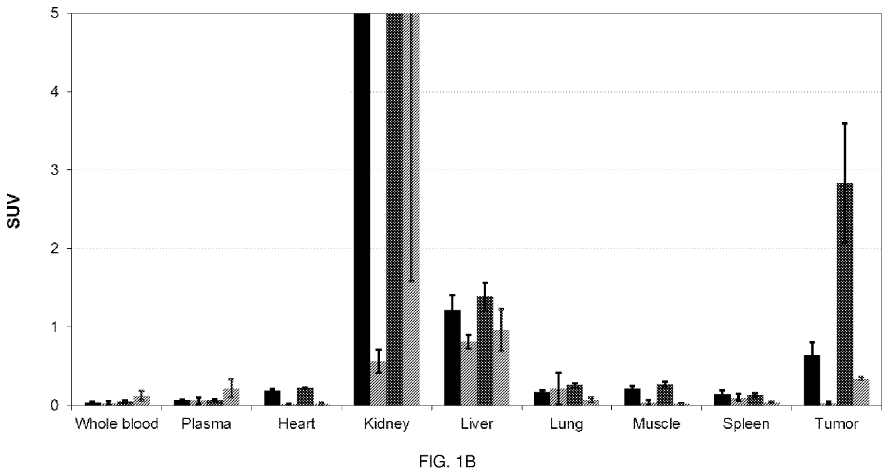

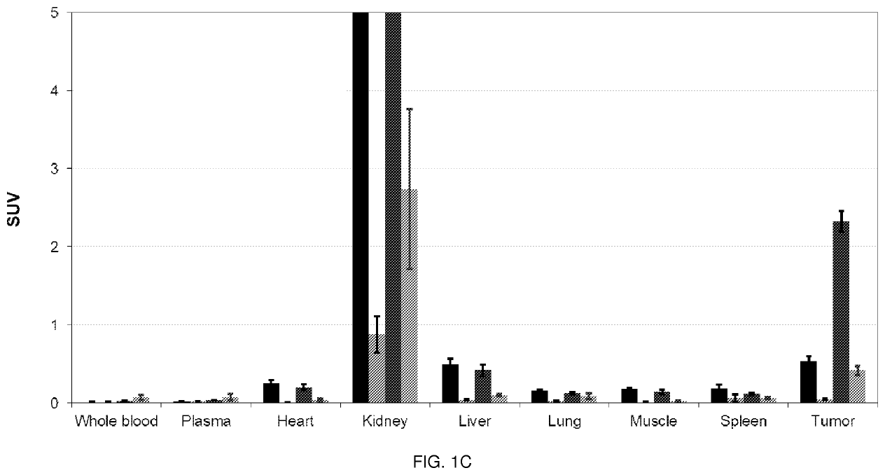

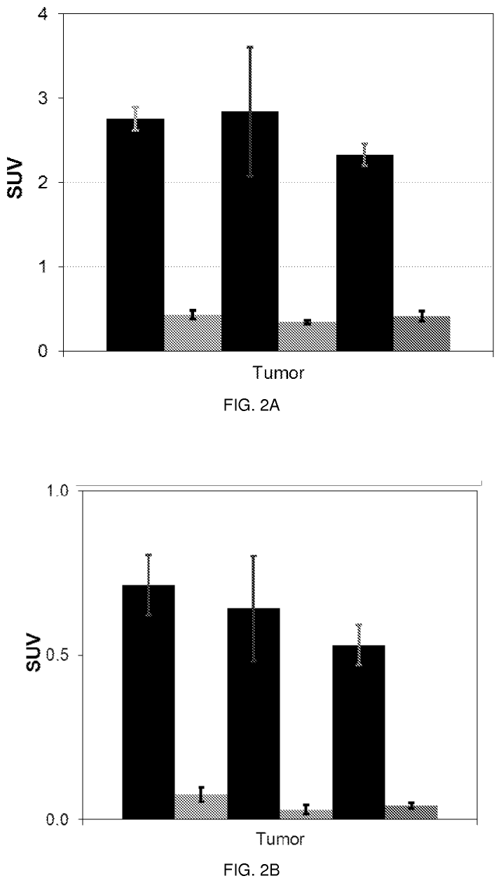

EXAMPLE. Ex vivo biodistribution study of compounds described herein under both baseline and competed conditions in nude mice bearing KB tumor xenografts on their left shoulder demonstrates a high and specific uptake in FR (+) tumors. Radiotracer levels of ¹⁸F-AIF-QC07017 and ¹⁸F-AIF-QC07043 were determined in whole blood, plasma, heart, kidney, liver, lung, muscle, spleen, KB xenograft tumor tissues and A549 xenograft tumor tissues (FIG. 1A, FIG. 1B, and FIG. 1C). The highest signal was observed in the kidneys. Accumulation was observed to a substantially less extent in the liver. Without being bound by theory, it is believed herein that the highest accumulation of radioactivity in kidneys, along with the relative low uptake of radiotracer in the hepatobiliary system i.e. liver, bile and intestine/feces supports that renal elimination is the predominant excretion pathway. Except for the kidneys, accumulation in KB xenograft tumor tissues was greatest, and significantly greater than in the liver. Accumulation in A549 xenograft tumor tissues was comparable to the liver. Accumulation in both KB xenograft tumor tissues and A549 xenograft tumor tissues was blocked under competition conditions with folic acid (FIG. 2A and FIG. 2B). The FR specificity of ¹⁸F-AIF-QC07017 and ¹⁸F-AIF-QC07043 was comparable to etarfolatide (EC20), a compound in clinical trials, in both KB xenograft tumor tissues and A549 xenograft tumor tissues.

Uptake in KB xenografts

| | | Uptake under competed |
|---|---|---|
| | Uptake | conditions |
| Example | (SUV) (±SEM) | (SUV) (±SEM) |
| ¹⁸F-AIF-QC07017 | 2.84 ± 0.76 | 0.34 ± 0.02 |
| ¹⁸F-AIF-QC07043 | 2.33 ± 0.13 | 0.41 ± 0.06 |
| ^{99m}Tc-EC20 | 2.75 ± 0.14 | 0.43 ± 0.05 |

P values: ^{99m}Tc-EC20 vs ¹⁸F-AIF-QC07043, p = 0.15; ¹⁸F-AIF-QC07017 vs ¹⁸F-AIF-QC07043, p = 0.48; EC20 vs ¹⁸F-AIF-QC07017, p = 0.85.

Uptake in A549 xenografts

| | | Uptake under competed |
|---|---|---|
| | Uptake | conditions |
| Example | (SUV) (±SEM) | (SUV) (±SEM) |
| ¹⁸F-AIF-QC07017 | 0.64 ± 0.16 | 0.03 ± 0.01 |
| ¹⁸F-AIF-QC07043 | 0.53 ± 0.06 | 0.04 ± 0.01 |
| ^{99m}Tc-EC20 | 0.71 ± 0.09 | 0.08 ± 0.02 |

P values: ^{99m}Tc-EC20 vs ¹⁸F-AIF-QC07043, p = 0.13; ¹⁸F-AIF-QC07017 vs ¹⁸F-AIF-QC07043, p = 0.50; ^{99m}Tc-EC20 vs ¹⁸F-AIF-QC07017, p = 0.74

EXAMPLE. In vitro evaluation of DUPA-EAOA-Phe-Phe-NOTA-⁶⁸Ga radiotracer (⁶⁸Ga-QC08009). ⁶⁷Ga has a longer half life than ⁶⁸Ga (about 3.3 days versus about 68 minutes, respectively). Thus, ⁶⁷Ga is used as a surrogate of ⁶⁸Ga for in vitro evaluation of Kd values and tissue imaging. It is to be understood that the in vitro evaluation of Kd values and tissue imaging observed for ⁶⁷Ga is predictive of ⁶⁸Ga. DUPA-EAOA-Phe-Phe-NOTA-⁶⁷Ga (⁶⁷Ga-NOTA-LC-PSMA2) was prepared in nearly quantitative radiochemical yield. In vitro study in both the PSMA (−) cell line (PC3) and the PSMA (+) cell lines (LnCaP and PIP—PC3) revealed a PSMA mediated high and specific uptake with a Kd=8.45±2.16 nM. PC3 is a PSMA (−) cell line; LnCap is a PSMA (+) cell line; and PIP—PC3 is a transfect cell line with higher PSMA expression. Uptake of ⁶⁸Ga-QC08009 by PC3 cells was minimal, and did not change when competed. Uptake of ⁶⁸Ga-QC08009 by LnCaP and PIP—PC3 was substantial, with PIP—PC3 cells showing the highest uptake. In both cases, Uptake of ⁶⁸Ga-QC08009 by LnCaP and PIP—PC3 is blocked by competing ligand. Compared to ⁶⁷Ga-DKFZ-PSMA11, an imaging agent in clinical trials, ⁶⁷Ga-NOTA-LC-PSMA2 demonstrated superior binding to PSMA (+) prostate cancer tissues.

EXAMPLE. In vivo PET imaging and BioD study of DUPA-EAOA-Phe-Phe-NOTA-⁶⁸Ga radiotracer (⁶⁸Ga-QC08009). In vivo micro-PET/CT scan with ⁶⁸Ga-NOTA-LC-PSMA2 radiotracer in mice carrying PSMA (+) LnCaP xenografts showed 4.29% ID uptake in PSMA (+) tumor. At 1 hour post-injection, most of the radiotracer was found in bladder. Without being bound by theory, it is believed herein that the data support that the primary elimination pathway is in urine. In addition, compared to other tissues, minor accumulation of radiotracer was observed in the kidneys. Without being bound by theory, it is believed herein that the relatively high PSMA expression in mouse kidneys, compared to other tissues, accounts at least in part for the minor accumulation of ⁶⁸Ga-NOTA-LC-PSMA2 radiotracer in kidneys.

1-19. (canceled)

20. A conjugate of the formula
B-L-P
or a pharmaceutically acceptable salt thereof, wherein B is a radical of a folate receptor binding ligand, L is a divalent linker, and P is a radical comprising a formula selected from the group consisting of

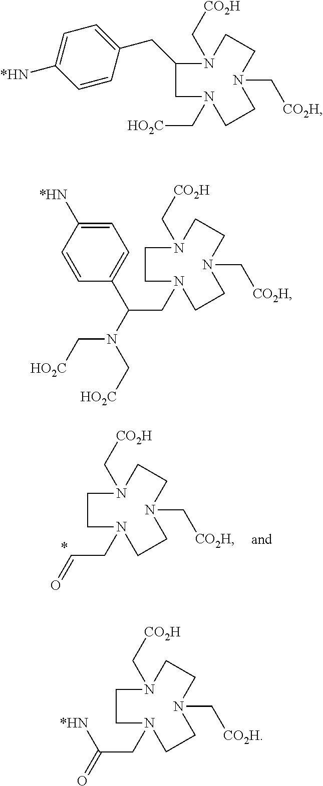

21. The conjugate of claim 20, wherein the radical of a folate receptor binding ligand is a radical of a folic acid.

22. The conjugate of claim 20, wherein the linker comprises lysine.

23. The conjugate of claim 20, wherein the linker comprises a polypeptide comprising lysine, arginine, aspartic acid, or a combination thereof.

24. The conjugate of claim 20, wherein the linker does not include a diradical of the formula *NH—(CH₂)₂—NH*.

25. The conjugate of claim 20, wherein the divalent linker L comprises *NH[(CH₂)₂O]ₙ*, *NH[(CH₂)₂O]ₙ—(CH₂)₂—C(O)*, *NH[(CH₂)₂O]ₙ—(CH₂)₂—C(O)NH*, *NH[(CH₂)₂O]ₙ—(CH₂)₂—C(O)NH—(CH₂)₂*, *NH[(CH₂)₂O]ₙ—(CH₂)₂—C(O)NH—(CH₂)₂NH*, *NH[(CH₂)₂O]ₙ—(CH₂)₂NH*, *NH(CH₂)₂O—(CH₂)₂NH*, *NH[(CH₂)₂O]₂—(CH₂)₂NH*, *NH[(CH₂)₂₀₁₆—(CH₂)₂NH*, or *NH[(CH₂)₂O]₁₂—(CH₂)₂NH*, where n is an integer from 1 to 12.

26. The conjugate of claim 20, wherein the divalent linker L comprises *NH[(CH₂)₂O]ₙ, *NH[(CH₂)₂O]ₙ—(CH₂)₂—C(O)*, *NH[(CH₂)₂O]ₙ—(CH₂)₂—C(O)NH*, *NH[(CH₂)₂O]ₙ—(CH₂)₂—C(O)NH—(CH₂)₂*, or *NH[(CH₂)₂O]ₙ—(CH₂)₂—C(O)NH—(CH₂)₂NH*, where n is an integer from 1 to 12.

27. The conjugate of claim 20, wherein the divalent linker L comprises *NH(CH₂)₂O—(CH₂)₂NH*, *NH[(CH₂)₂O]₂—(CH₂)₂NH*, *NH[(CH₂)₂O]₆—(CH₂)₂NH*, or *NH[(CH₂)₂O]₁₂—(CH₂)₂NH *.

28. The conjugate of claim 20, which comprises folate-polyethylene glycol (PEG).

29. The conjugate of claim 20, comprising the formula 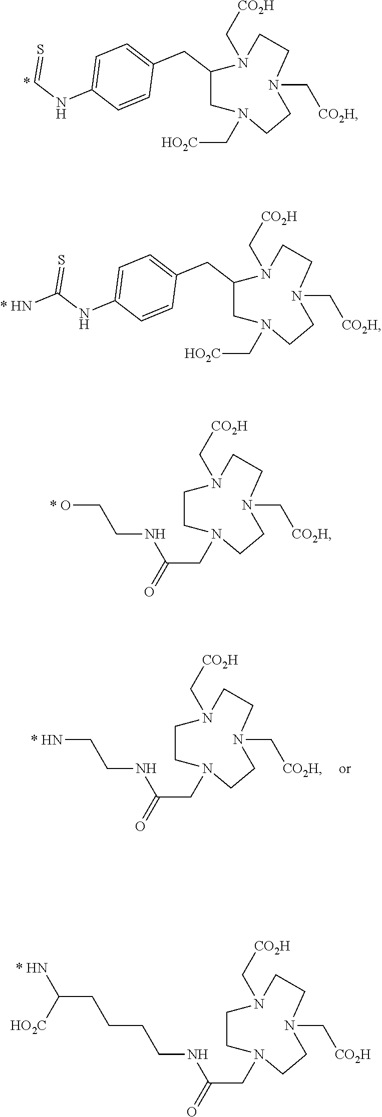
coordinated to a radionuclide or radionuclide-containing group.

30. The conjugate of claim 20, comprising the formula 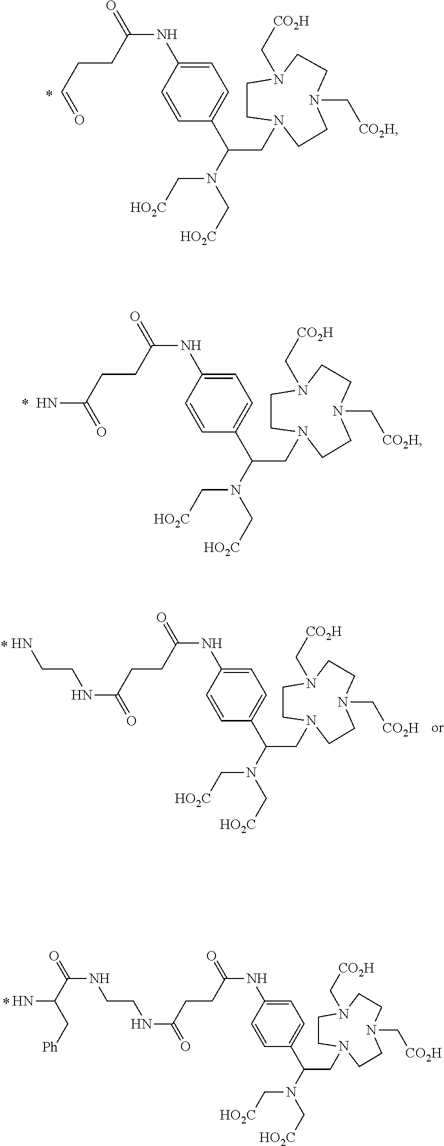 [SMILES: O=CCCC(=O)Nc1ccc(C(CN2CCN(CC(=O)O)CCN(CC(=O)O)CC2)N(CC(=O)O)CC(=O)O)cc1 ; NC(=O)CCC(=O)Nc1ccc(C(CN2CCN(CC(=O)O)CCN(CC(=O)O)CC2)N(CC(=O)O)CC(=O)O)cc1 ; NCCNC(=O)CCC(=O)Nc1ccc(C(CN2CCN(CC(=O)O)CCN(CC(=O)O)CC2)N(CC(=O)O)CC(=O)O)cc1 ; NC(Cc1ccccc1)C(=O)NCCNC(=O)CCC(=O)Nc1ccc(C(CN2CCN(CC(=O)O)CCN(CC(=O)O)CC2)N(CC(=O)O)CC(=O)O)cc1]
coordinated to a radionuclide or radionuclide-containing group.

31. The conjugate of claim 20, comprising the formula

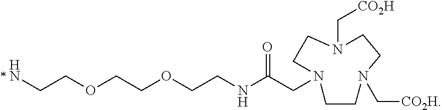

32. The conjugate of claim 20, wherein the conjugate is

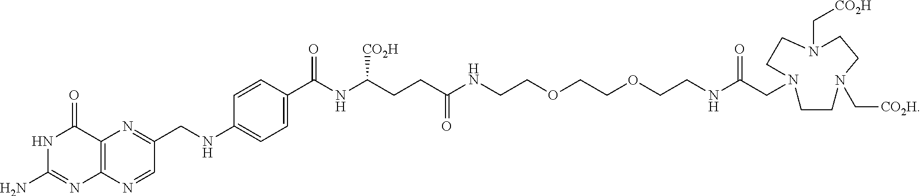 [SMILES: Nc1nc2ncc(CNc3ccc(C(=O)N[C@@H](CCC(=O)NCCOCCOCCNC(=O)CN4CCN(CC(=O)O)CCN(CC(=O)O)CC4)C(=O)O)cc3)nc2c(=O)[nH]1]

33. The conjugate of claim 20, wherein the conjugate is

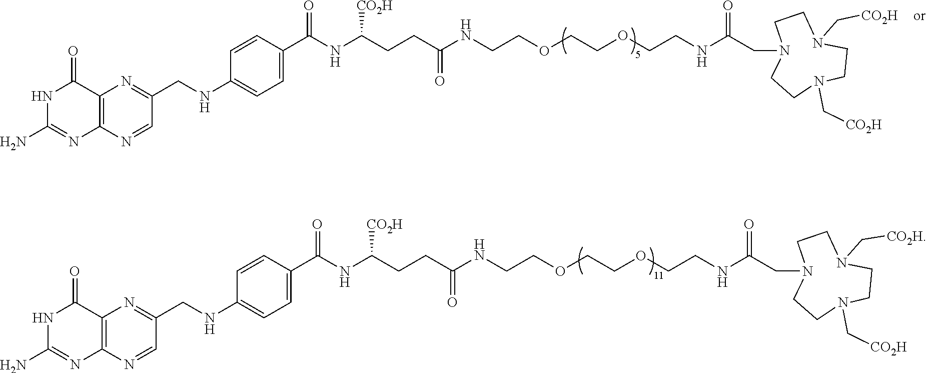

34. The conjugate of claim 20, comprising the formula

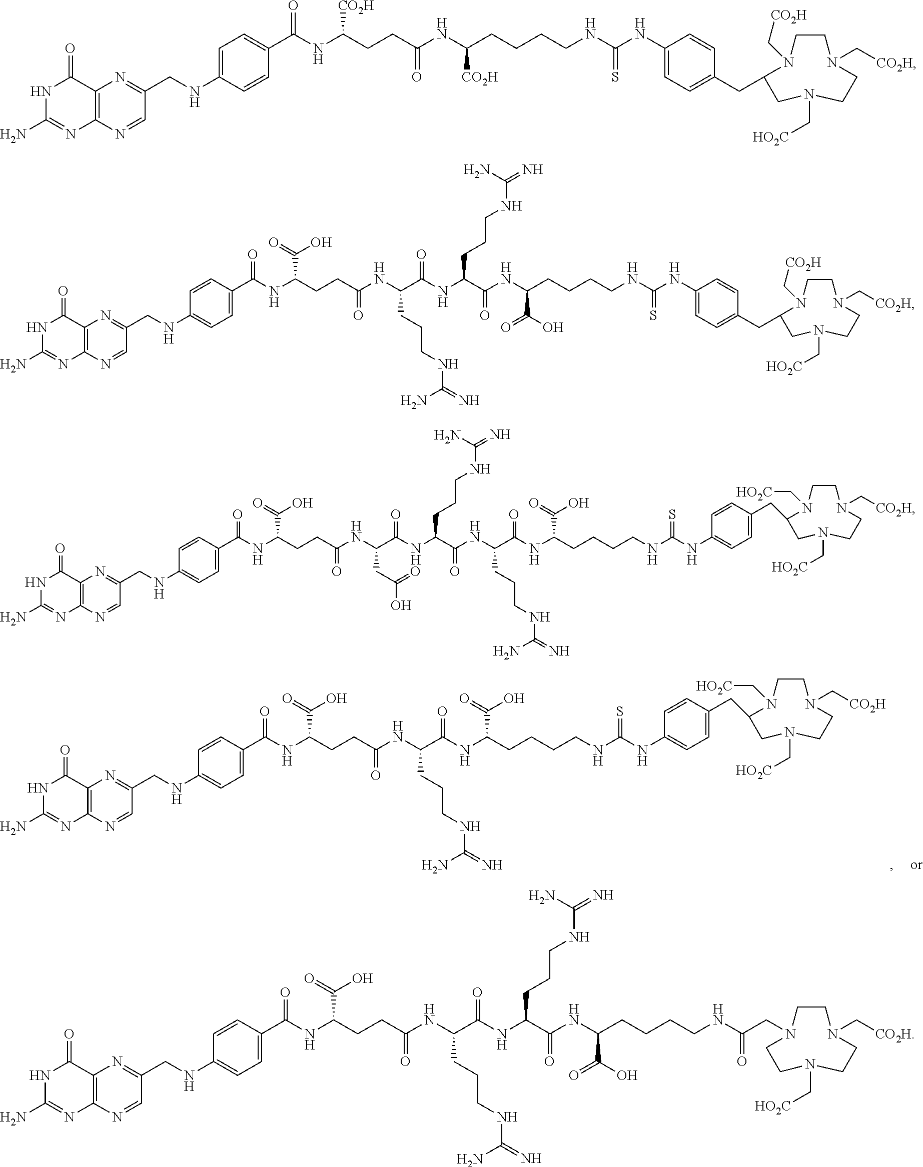

35. The conjugate of claim 20, further comprising a radionuclide or radionuclide-containing group.

36. The conjugate of claim 20, wherein the radionuclide or the radionuclide in the radionuclide-containing group is selected from the group consisting of ⁶⁸Ga, ⁶⁶Ga, ¹⁸F, ¹⁷⁷Lu, ⁹⁰Y, ⁶⁴Cu, ⁶¹ Cu, ⁸⁹Zr, ⁴⁵Ti, ⁵¹Mn, ⁶³Zn, ⁸²Rb, ¹²⁴I, ¹³¹I, ⁸⁹Sr, ¹⁵³Sm, ¹¹C, ¹³N, and 150.

37. The conjugate of claim 20, wherein the conjugate is 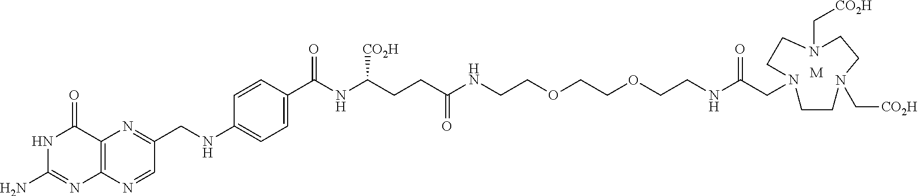
wherein M is Al-¹⁸F or ⁶⁸Ga.

38. A pharmaceutical composition comprising one or more of the conjugates of claim 20, in combination with one or more carriers, diluents, or excipients, or a combination thereof.

39. A method of imaging a disease in a host animal, comprising the step of administering to the host animal a diagnostically effective amount of one or more of the conjugates of claim 20 and imaging the host animal.
